(19) United States
(12) Patent Application Publication
(10) Pub. No.: US 2026/0193656 A1
(43) Pub. Date: Jul. 9, 2026

(54) GUIDE RNA COMPOSITIONS

(71) Applicant: nChroma Bio, Inc., Boston, MA (US)

(72) Inventors: Ari FRIEDLAND, Cambridge, MA (US); Vic Myer, Arlington, MA (US)

(21) Appl. No.: 19/419,592

(22) Filed: Dec. 15, 2025

Related U.S. Application Data
(63) Continuation of application No. PCT/US2024/035873, filed on Jun. 27, 2024.
(60) Provisional application No. 63/511,624, filed on Jun. 30, 2023.

(51) Int. Cl. C12N 15/113; A61K 31/712; A61K 31/7125; A61K 48/00; C12N 9/22

(52) U.S. Cl. CPC C12N 15/113; A61K 31/712; A61K 31/7125; A61K 48/005; C12N 9/222; C12N 2310/20; C12N 2310/315; C12N 2310/321; C12N 2310/531

(57) ABSTRACT

Disclosed herein are modified single guide RNAs having improved activity in gene editing methods.

### CROSS REFERENCE

This application is a continuation of International Application No. PCT/US2024/035873, filed on Jun. 27, 2024, which claims the benefit of U.S. Provisional Application No. 63/511,624, filed Jun. 30, 2023, each of which is incorporated herein by reference in its entirety.

### SEQUENCE LISTING

The instant application contains a Sequence Listing which has been submitted electronically in XML file format and is hereby incorporated by reference in its entirety. Said XML copy, created on Aug. 29, 2024, is named 59073-738_601_SL.xml and is 140,476 bytes in size.

### BACKGROUND

Oligonucleotides, such as guide RNAs (gRNAs) of CRISPR/Cas system for gene editing, can be subjected to degradation in cells by endonuclease or exonuclease cleavage. There is a need to prevent degradation of gRNAs and improve stability of gRNAs to enhance the gene editing efficiency.

### SUMMARY

Described herein is a single guide RNA (sgRNA) comprising a tracr sequence and a spacer sequence, (i) wherein the tracr sequence comprises an upper stem, a hairpin region 1, and a hairpin region 2, and wherein (a) at least 50% of the nucleotides in the upper stem loop are 2′-OMe modified, and at least one nucleotide in the upper stem region is not 2′-O-Me modified; and (b) at least 50% of the nucleotides in the hairpin region are 2′-O-Me modified, and at least one nucleotide in the hairpin region is not 2′-O-Me modified; or (ii) wherein the spacer sequence comprises a 5′ end, and wherein the spacer sequence comprises not more than one PS bond within the first three seven nucleotides of the 5′ end.

In some embodiments, each nucleotide in the upper stem region is modified with 2′-O-Me except the first nucleotide of the upper stem region in the 5′ end to 3′ end direction. In some embodiments, each nucleotide in the upper stem region is modified with 2′-O-Me except the last nucleotide of the upper stem region in the 5′ end to 3′ end direction. In some embodiments, the upper stem region further comprises nucleotides modified with 2′-deoxy (2′-H), 2-MOE, 2′-F, 2′-NH2, 2′-arabinosyl (2-arabino) nucleotide, 2-F-arabinosyl (2′-F-arabino) nucleotide, 2′-locked nucleic acid (LNA) nucleotide, 2′-unlocked nucleic acid (ULNA) nucleotide, a sugar in L form (L-sugar), 4′-thioribosyl nucleotide, or any combination thereof. In some embodiments, at least 50%, 60%, 70%, 80%, or 90%, or 100% of nucleotides in the upper stem region are modified. In some embodiments, at least 82.5% of the nucleotides in the upper stem region are modified. In some embodiments, each nucleotide in the upper stem region is modified with 2′-O-Me.

In some embodiments, the upper stem region comprises a linkage modification. In some embodiments, the linkage modification comprises phosphorothioate, phosphonoacetate, thiophosphonoacetate, methylphosphonoate-P(CH3), boranophosphonate, phosphorodithioate, or any combination thereof.

In some embodiments, the spacer sequence comprises not more than one PS bond within the first three nucleotides at the 5′ end.

In some embodiments, each nucleotide in the hairpin region 2 is modified with 2′-O-Me except the first nucleotide in the 5′ end to 3′ end direction. In some embodiments, the hairpin region 2 further comprises nucleotides modified with 2′-deoxy (2′-H), 2-MOE, 2′-F, 2′-NH2, 2′-arabinosyl (2-arabino) nucleotide, 2-F-arabinosyl (2′-F-arabino) nucleotide, 2′-locked nucleic acid (LNA) nucleotide, 2′-unlocked nucleic acid (ULNA) nucleotide, a sugar in L form (L-sugar), 4′-thioribosyl nucleotide, or any combination thereof. In some embodiments, at least 50%, 60%, 70%, 80%, or 90%, or 100% of nucleotides in the hairpin region 2 are modified. In some embodiments, at least 82.5% of nucleotides in the hairpin region 2 are modified. In some embodiments, each nucleotide in the hairpin region 2 is modified with 2′-O-Me.

In some embodiments, the hairpin region 2 comprises a linkage modification. In some embodiments, the linkage modification comprises phosphorothioate, phosphonoacetate, thiophosphonoacetate, methylphosphonoate-P(CH3), boranophosphonate, phosphorodithioate, or any combination thereof.

In some embodiments, the 5′ end of the 5′ terminus comprises the PS linkage between the first and second nucleotides. In some embodiments, the 5′ terminus comprises nucleotides modified with 2′-O-Me, 2′-deoxy (2′-H), 2-MOE, 2′-F, 2′-NH2, 2′-arabinosyl (2-arabino) nucleotide, 2-F-arabinosyl (2′-F-arabino) nucleotide, 2′-locked nucleic acid (LNA) nucleotide, 2′-unlocked nucleic acid (ULNA) nucleotide, a sugar in L form (L-sugar), 4′-thioribosyl nucleotide, or any combination thereof. In some embodiments, at least 50%, 60%, 70%, 80%, or 90%, or 100% nucleotides of the 5′ terminus are modified. In some embodiments, the 5′ end of the 5′ terminus comprises 1, 2, 3, 4, 5, 6, 7, 8, 9, or 10 nucleotides modified with 2′-O-Me, 2′-deoxy (2′-H), 2-MOE, 2′-F, 2′-NH2, 2′-arabinosyl (2-arabino) nucleotide, 2-F-arabinosyl (2′-F-arabino) nucleotide, 2′-locked nucleic acid (LNA) nucleotide, 2′-unlocked nucleic acid (ULNA) nucleotide, a sugar in L form (L-sugar), 4′-thioribosyl nucleotide, or any combination thereof. In some embodiments, nucleotides of the 5′ terminus other than the first 1, 2, 3, 4, 5, 6, 7, 8, 9, or 10 nucleotides are not modified.

In some embodiments, the 5′ terminus comprises a linkage modification. In some embodiments, the linkage modification comprises phosphorothioate, phosphonoacetate, thiophosphonoacetate, methylphosphonoate-P(CH3), boranophosphonate, phosphorodithioate, or any combination thereof.

In some embodiments, the hairpin region 1 comprises nucleotides modified with 2′-O-Me, 2′-deoxy (2′-H), 2-MOE, 2-F, 2′-NH2, 2′-arabinosyl (2-arabino) nucleotide, 2′-F-arabinosyl (2′-F-arabino) nucleotide, 2′-locked nucleic acid (LNA) nucleotide, 2′-unlocked nucleic acid (ULNA) nucleotide, a sugar in L form (L-sugar), 4′-thioribosyl nucleotide, or any combination thereof. In some embodiments, at least 50%, 60%, 70%, 80%, or 90%, or 100% nucleotides of the hairpin region 1 are modified. In some embodiments, each nucleotide of the hairpin region 1 is modified with 2′-O-Me, 2′-deoxy (2′-H), 2-MOE, 2-F, 2′-NH2, 2′-arabinosyl (2-arabino) nucleotide, 2′-F-arabinosyl (2′-F-arabino) nucleotide, 2′-locked nucleic acid (LNA) nucleotide, 2′-unlocked nucleic acid (ULNA) nucleotide, a sugar in L form (L-sugar), 4′-thioribosyl nucleotide, or any combination thereof. In some embodiments, each nucleotide in the hairpin region 1 is modified with 2′-O-Me.

In some embodiments, the hairpin region 1 comprises a linkage modification. In some embodiments, the linkage modification comprises phosphorothioate, phosphonoacetate, thiophosphonoacetate, methylphosphonoate-P(CH3), boranophosphonate, phosphorodithioate, or any combination thereof.

In some embodiments, the sgRNA further comprises a 3′ terminus region comprising a modification. In some embodiments, the 3′ terminus comprises one or more nucleotides modified with 2′-O-Me, 2′-deoxy (2′-H), 2-MOE, 2′-F, 2′-NH2, 2′-arabinosyl (2-arabino) nucleotide, 2′-F-arabinosyl (2′-F-arabino) nucleotide, 2′-locked nucleic acid (LNA) nucleotide, 2′-unlocked nucleic acid (ULNA) nucleotide, a sugar in L form (L-sugar), 4′-thioribosyl nucleotide, and combinations thereof. In some embodiments, at least two of the last four nucleotides at a 3′ end of the 3′ terminus are modified. In some embodiments, last four nucleotides at a 3′ end of the 3′ terminus are linked with PS bonds.

In some embodiments, the 3′ terminus comprises a linkage modification. In some embodiments, the linkage modification comprises phosphorothioate, phosphonoacetate, thiophosphonoacetate, methylphosphonoate-P(CH3), boranophosphonate, phosphorodithioate, or any combination thereof.

In some embodiments, the sgRNA further comprises a lower stem region comprising a modification.

In some embodiments, the sgRNA further comprises a bulge region between the upper stem region and the lower stem region, wherein the bulge region comprises a modification.

In some embodiments, the sgRNA further comprises a nexus region between the hairpin region 1 and the lower stem region, wherein the nexus region comprises a modification.

In some embodiments, the modification comprises one or more modifications selected from the group consisting of: 2′-O-Me, 2′-deoxy (2′-H), 2-MOE, 2′-F, 2′-NH2, 2′-arabinosyl (2-arabino) nucleotide, 2′-F-arabinosyl (2′-F-arabino) nucleotide, 2′-locked nucleic acid (LNA) nucleotide, 2′-unlocked nucleic acid (ULNA) nucleotide, a sugar in L form (L-sugar), 4′-thioribosyl nucleotide, and combinations thereof. In some embodiments, the modification comprises 2′-O-Me, 2′-F, or 2′-MOE. In some embodiments, the modification comprises 2′O-Me. In some embodiments, the modification comprises a linkage modification. In some embodiments, the linkage modification is selected from the group consisting of: phosphorothioate, phosphonoacetate, thiophosphonoacetate, methylphosphonoate-P(CH3), boranophosphonate, and phosphorodithioate.

In some embodiments, the 5′ terminus targets hPCSK9. In some embodiments, the 5′ terminus comprises a sequence set forth in any one of SEQ ID NOs: 6-14.

In some embodiments, the sgRNA comprises (a) and (b).

In some embodiments, the upper stem region comprises nucleotides modified with 2′-O-Me, and wherein the first nucleotide of the upper stem region in the 5′ end to 3′ end direction is not modified with 2′O-Me. In some embodiments, each nucleotide in the upper stem region is modified with 2′-O-Me except the first nucleotide of the upper stem region in the 5′ end to 3′ end direction. In some embodiments, the upper stem region comprises nucleotides modified with 2′-O-Me, and wherein the last nucleotide of the upper stem region in the 5′ end to 3′ end direction is not modified with 2′O-Me. In some embodiments, each nucleotide in the upper stem region is modified with 2′-O-Me except the first nucleotide of the upper stem region in the 5′ end to 3′ end direction.

In some embodiments, the first nucleotide of the hairpin region 2 in the 5′ end to 3′ end direction is not modified. In some embodiments, each nucleotide in the hairpin region 1 is modified with 2′-O-Me.

In some embodiments, the sgRNA further comprises a 3′ terminus that comprises a modification. In some embodiments, last four nucleotides at a 3′ end of the 3′ terminus are linked with a PS linkage. In some embodiments, last four nucleotides of the 3′ end of the 3′ terminus are modified with 2′-O-Me.

In some embodiments, a 5′ end of the 5′ terminus comprises first four nucleotides linked with a PS linkage. In some embodiments, the 5′ terminus targets hPCSK9. In some embodiments, the 5′ terminus comprises a sequence set forth in any one of SEQ ID NOs: 6-10.

In some embodiments, the sgRNA comprises a sequence set forth in any one of SEQ ID NOs: 18-22. In some embodiments, the sgRNA comprises a sequence set forth in any one of SEQ ID NOs: 23-27. In some embodiments, the sgRNA comprises a sequence set forth in any one of SEQ ID NOs: 32 or 33.

In some embodiments, the sgRNA comprises the feature of (c).

In some embodiments, each nucleotide in the upper stem region is modified with 2′-O-Me.

In some embodiments, each nucleotide in the hairpin region 1 is modified with 2′-O-Me.

In some embodiments, each nucleotide in the hairpin region 2 is modified with 2′-O-Me.

In some embodiments, the sgRNA further comprises a 3′ terminus that comprises a modification. In some embodiments, last four nucleotides at a 3′ end of the 3′ terminus are linked with a PS linkage. In some embodiments, last four nucleotides of the 3′ end of the 3′ terminus are modified with 2′-O-Me.

In some embodiments, the 5′ terminus targets hPCSK9. In some embodiments, the 5′ terminus comprises a sequence set forth in any one of SEQ ID NOs: 11-14.

In some embodiments, the sgRNA comprises a sequence set forth in any one of SEQ ID NOs: 28-31. In some embodiments, the sgRNA comprises a sequence set forth in SEQ ID NO: 34.

Also described herein is a single guide RNA (sgRNA) comprising: (i) an upper stem region, a hairpin region 1, a hairpin region 2, wherein (a) the upper stem region comprises nucleotides modified with 2′-O-Me; (b) the hairpin region 1 and the hairpin region 2 comprise nucleotides modified with 2′-O-Me; or (c) both (a) and (b); and (ii) a 5′ terminus, wherein the 5′ terminus targets hPCSK9.

In some embodiments, the 5′ terminus comprises any of SEQ ID NOs:1-14.

Also described herein is a compound of Formula (I):

X_{A1}X_{A2}X_{A3}(Xᵢ)_{M}X_{B1}X_{B2}X_{B3}X_{B4}X_{B5}X_{B6}X_{C1}X_{C2}X_{D1}X_{D2}X_{D3}
X_{D4}X_{D5}X_{D6}X_{D7}X_{D8}X_{D9}X_{D10}X_{D11}X_{D12}X_{C3}X_{C4}X_{C5}X_{C6}X_{B7}X_{B8}
X_{B9}X_{B10}X_{B11}X_{B12}X_{E1}X_{E2}X_{E3}X_{E4}X_{E5}X_{E6}X_{E7}X_{E8}X_{E9}X_{E10}X_{E11}
X_{E12}X_{E13}X_{E14}X_{E15}X_{E16}X_{E17}X_{E18}X_{F1}X_{F2}X_{F3}X_{F4}X_{F5}X_{F6}X_{F7}
X_{F8}X_{F9}X_{F10}X_{F11}X_{F12}X_{G1}X_{H1}X_{H2}X_{H3}X_{H4}X_{H5}X_{H6}X_{H7}X_{H8}X_{H9}
X_{H10}X_{H11}X_{H12}X_{H13}X_{H14}X_{H15}X_{J1}X_{J2}X_{J3}X_{J4}

(I), wherein: each of X_{B6}, X_{B8}, X_{B9}, X_{B10}, X_{B11}, X_{C2}, X_{C3}, X_{C4}, X_{E1}, X_{E2}, X_{E7}, X_{E15}, and X_{E18} is

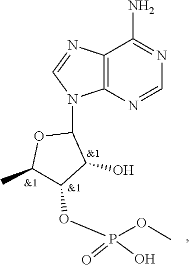

each of X_{E5}, X_{E10}, X_{W11}, X_{E17}, is

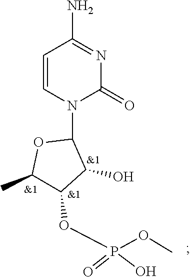

each of X_{B1}, X_{C1}, X_{C5}, X_{E1}, X_{E4}, X_{E8} and X_{E12} is

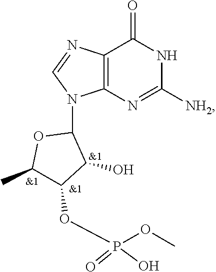 [SMILES: COP(=O)(O)O[C@@H]1[C@@H](C)OC(n2cnc3c(=O)[nH]c(N)nc32)[C@@H]1O]

and
each of X_{B2}, X_{B3}, X_{B4}, X_{B5}, X_{B7}, X_{B12}, X_{C6}, X_{E6}, X_{E9}, X_{E13}, X_{E14}, X_{E16} is

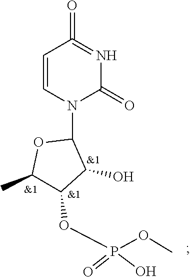

wherein: each of X_{F1}, X_{F6}, X_{F7}, X_{F8}, X_{F9}, X_{F10}, is

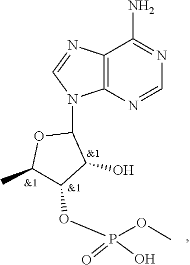

X_{F2} is

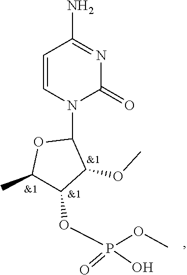

each of X_{F5}, X_{F11}, X_{G1} is

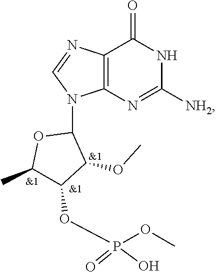 [SMILES: CO[C@H]1C(n2cnc3c(=O)[nH]c(N)nc32)O[C@H](C)[C@H]1OP(=O)(O)OC]

and
each of X_{F3}, X_{F4}, X_{F12} is

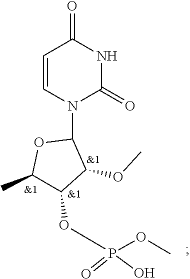

wherein: each of X_{D4}, X_{D6}, X_{D7}, X_{D8}, X_{D10}, X_{H3}, and X_{H7} is

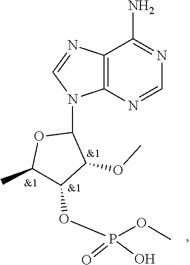

each of X_{D2}, X_{H2}, X_{H4}, X_{H5}, X_{H10}, and X_{H15} is

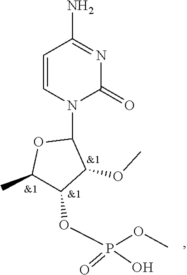

each of X_{D5}, X_{D11}, X_{H6}, X_{H8}, X_{H11}, X_{H12}, and X_{H14} is

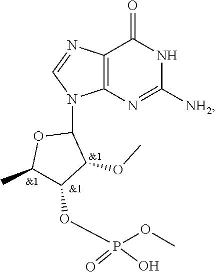 [SMILES: CO[C@H]1C(n2cnc3c(=O)[nH]c(N)nc32)O[C@H](C)[C@H]1OP(=O)(O)OC]

each of X_{D3}, X_{D9}, X_{H9}, and X_{H13} is

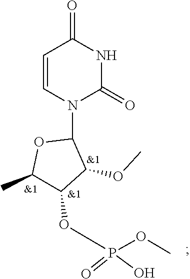

each of X_{J1} and X_{J2}, and X_{J3} is

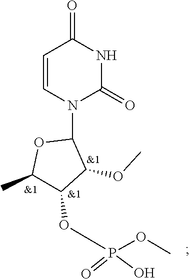

and

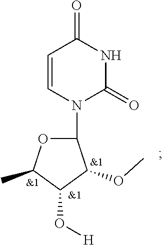

wherein; X_{A1} is

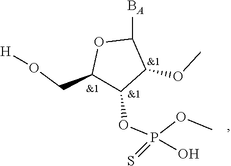

M is 14, 15, 16, 17, 18, 19, 20, or 21,
Xᵢ is

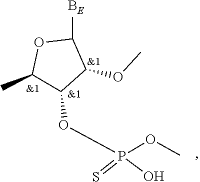

wherein:
(a) X_{A2} and X_{A3} are both

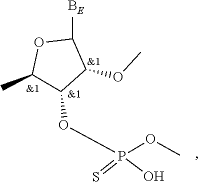

X_{D1} is

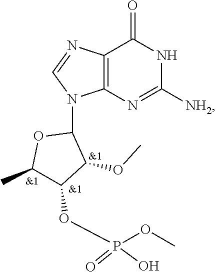 [SMILES: CO[C@H]1C(n2cnc3c(=O)[nH]c(N)nc32)O[C@H](C)[C@H]1OP(=O)(O)OC]

X_{D12} is

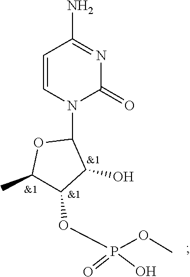

and

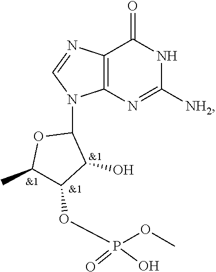 [SMILES: COP(=O)(O)O[C@@H]1[C@@H](C)OC(n2cnc3c(=O)[nH]c(N)nc32)[C@@H]1O]

(b) X_{A2} and X_{A3} are both

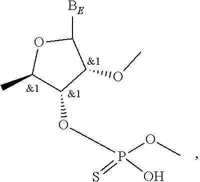

X_{D1} is

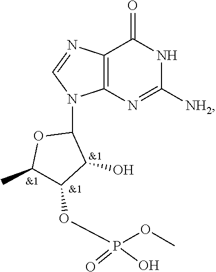 [SMILES: COP(=O)(O)O[C@@H]1[C@@H](C)OC(n2cnc3c(=O)[nH]c(N)nc32)[C@@H]1O]

X_{D12} is

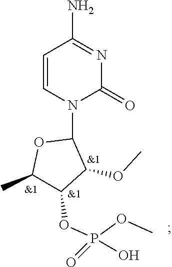

and X_{D12} is

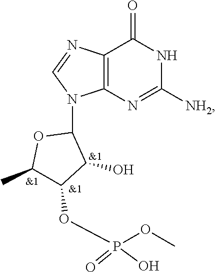 [SMILES: COP(=O)(O)O[C@@H]1[C@@H](C)OC(n2cnc3c(=O)[nH]c(N)nc32)[C@@H]1O]

or
(c) X_{A2} and X_{A3} are both

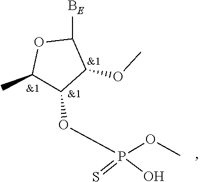

X_{D1} and X_{H1} are both

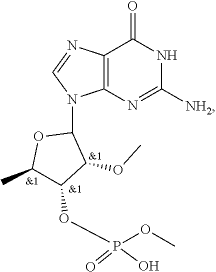 [SMILES: CO[C@H]1C(n2cnc3c(=O)[nH]c(N)nc32)O[C@H](C)[C@H]1OP(=O)(O)OC]

and X_{D12} is

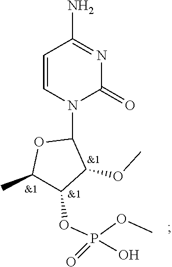

wherein each of B_{A}, B_{B}, B_{C}, B_{D}, and B_{E} is independently selected from the group consisting of:

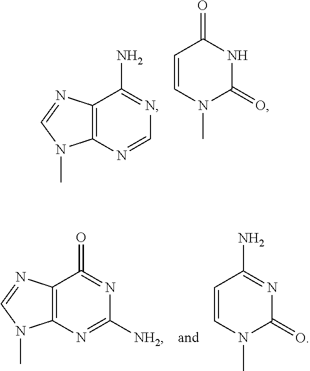

Also described herein is an epigenetic system comprising a nuclease, and the sgRNA of any one of the embodiments described herein or the compound described herein.

Also described herein is a kit comprising a nuclease, the sgRNA of any one of the embodiments described herein or the compound described herein, and instructions for use thereof.

Also described herein is a method, comprising administering the sgRNA of any one of the embodiments described herein or the compound described herein to a cell.

Also described herein is a method, comprising administering the sgRNA of any one of the embodiments described herein or the compound described herein to a subject in need thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates the different regions (e.g., 5′ terminus, lower stem, bulge, upper stem, bulge hairpin region 1, hairpin region 2, 3′ terminus) of a single guide RNA (sgRNA).

FIGS. 2-3 illustrate particular embodiments of the present disclosure. Arrows indicate nucleotides that are not 2′-OMe modified.

FIG. 4 illustrates a particular embodiment of the present disclosure. Arrows indicate nucleotides not bound to the next through a phosphorothioate bond.

### DETAILED DESCRIPTION

### Definitions

As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The term “nucleic acid,” “nucleic acid molecule,” or “polynucleotide,” or any grammatical equivalents, can refer to a polymeric compound comprising covalently linked nucleotides. The term “nucleic acid” includes polyribonucleic acid (RNA) and poly deoxyribonucleic acid (DNA), both of which may be single- or double-stranded. DNA includes, but is not limited to, complimentary DNA (cDNA), genomic DNA, plasmid or vector DNA, and synthetic DNA.

The term “RNA guide” or “RNA guide sequence” or “guide RNA” refers to any RNA molecule that can modulate the targeting of a polypeptide to a target nucleic acid (e.g., a sequence of a PCSK9). For example, a guide RNA can be a molecule that recognizes (e.g., binds specifically to) a target nucleic acid or sequence. A guide RNA can be made to be complementary to a specific target sequence. A guide RNA guide comprises a DNA targeting sequence (“a spacer sequence” or “SPACR”) that is complementary to a specific DNA target sequence and a direct repeat sequence. A guide RNA also refers collectively to either a single guide RNA (sgRNA), a tracrRNA, or a CRISPR RNA (crRNA). The crRNA and tRNA can be associated on one RNA molecule (sgRNA) or in two separate molecules. The tracrRNA sequences can be naturally occurring, or the tracrRNA sequence can include variations compared to the naturally occurring sequences.

The term “target gene,” “target” or “target sequence” refers to a nucleic acid sequence to which an RNA guide (e.g., gRNA, sgRNA) specifically binds. In some embodiments, the DNA targeting sequence (e.g., spacer sequence) of a gRNA binds specifically to a target DNA sequence. In the case of a double-stranded target, the gRNA binds to a first strand of the target, and a protospacer adjacent motif (PAM) sequence is present in the second, complementary strand.

The term “identity” or “percent identity” between two or more nucleotide or amino acid sequences can be determined by aligning the sequences for optimal comparison purposes (e.g., gaps can be introduced in the sequence of a first sequence). The nucleotides at corresponding positions can then be compared, and the percent identity between the two sequences can be a function of the number of identical positions shared by the sequences (i.e., % identity=# of identical positions/total # of positions×100). The percent identity between the two sequences may be a function of the number of identical positions shared by the sequences, taking into account the number of gaps, and the length of each gap, which need to be introduced for optimal alignment of the two sequences. In some cases, the length of a sequence aligned for comparison purposes is at least about 30%, at least about 40%, at least about 50%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 95%, or 100% of the length of the reference sequence. A BLAST® search can determine homology between two sequences. The actual comparison of the two sequences can be accomplished by well-known methods, for example, using a mathematical algorithm. A non-limiting example of such a mathematical algorithm can be those described in Karlin, S. and Altschul, S., Proc. Natl. Acad. Sci. USA, 90-5873-5877 (1993). Such an algorithm can be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. Other non-limiting examples include the algorithm of Myers and Miller, CABIOS (1989), ADVANCE, ADAM, BLAT, and FASTA.

“Hairpin” refers to a loop of nucleic acids that is created when a nucleic acid strand folds and forms base pairs with another section of the same strand. A hairpin can form a structure that comprises a loop or a U-shape. A hairpin can comprise stem or stem loop structures. A hairpin can be comprised of an RNA loop. A hairpin can be formed with two complementary sequences in a single nucleic acid molecule bound together, with a folding or wrinkling of the molecule.

“Regions” refers to conserved groups of nucleic acids. Regions may also be referred to as “modules” or “domains.” Regions of a gRNA may perform particular functions, e.g., in directing endonuclease activity of the RNP, for example as described in Briner A E et al., Molecular Cell 56: 333-339 (2014). Regions of a gRNA are described in Tables 1-3.

“Ribonucleoprotein” (RNP) or “RNP complex” as used herein describes a gRNA, for example, together with a nuclease, such as a Cas protein. In some embodiments, the RNP comprises Cas9 and gRNA.

“Stem loop” refers to a secondary structure of nucleotides that form a base-paired “stem” that ends in a loop of unpaired nucleic acids. A stem can be formed when two regions of the same nucleic acid strand are at least partially complementary in sequence when read in opposite directions. “Loop” as used herein describes a region of nucleotides that do not base pair (i.e., are not complementary) that may cap a stem.

“Editing efficiency” or “editing percentage” or “percent editing” refers the total number of sequence reads with insertions or deletions of nucleotides into the target region of interest over the total number of sequence reads following cleavage by a Cas RNP.

The term “DNA binding domain” refers to DNA-binding domains from proteins selected from the family of CRISPR proteins, TAL proteins, zinc fingers, and other transcriptional regulators, their homologs, orthologs, and mutants, which maintain or enhance the basic function of DNA-binding proteins.

The term “repressor domain” or “transcriptional repressor domain” refers to a transcription repression protein or a portion thereof, such as a transcription factor, which can complex with one or more DNA binding domains to act as a negative regulatory domain. Repressor domains block the recruitment of RNA polymerase in order to suppress the transcription of certain genes. A repressor domain allows for the precise control of gene expression by inhibiting the activation of transcription through interaction with other cellular components such as but not limited to basal transcription factors, effector molecules, activator or coactivator proteins, repressors, and corepressors.

The term “KRAB” refers to Krüppel associated box, a transcription repression protein domain. KRAB refers to homologs, orthologs and mutants of the KRAB domain that have a conserved or enhanced basic function of inhibiting the transcription of structural genes. A KRAB domain is one of a group of transcriptional repression domains present in approximately 400 human zinc finger protein-based transcription factors. KRAB domains typically include about 45 to about 75 amino acid residues. A description of KRAB domains, including their function and use, may be found, for example, in Ecco, G., Imbeault, M., Trono, D., KRAB zinc finger proteins, Development 144, 2017.

The term “DNMT” refers to a DNA methyltransferase. As used herein, this term encompasses an enzyme that catalyzes the transfer of a methyl group to DNA such as canonical cytosine-5 DNMTs that catalyze the addition of methyl groups to genomic DNA (e.g., DNMT1, DNMT3A, DNMT3B, and DNMT3C). This term also encompasses non-canonical family members that do not catalyze methylation themselves but that recruit or activate catalytically active DNMTs, with non-limiting examples of such DNA methyltransferases including DNMT3L. See, e.g., Lyko, Nat Review. (2018) 19:81-92. Unless otherwise indicated, a DNMT domain may refer to a polypeptide domain derived from a catalytically active DNMT (e.g., DNMT1, DNMT3A, and DNMT3B) or from a catalytically inactive DNMT (e.g., DNMT3L).

The terms “subject,” “patient,” or “individual” are often used interchangeably herein. A “subject” may be a biological entity containing expressed genetic materials. The biological entity can be a plant, animal, or microorganism, including, for example, bacteria, viruses, fungi, and protozoa. The subject can be tissues, cells, and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease. A subject may or may not have been exposed to a pathogen of interest as described herein, and may be symptomatic or asymptomatic for a disease or condition associated with infection of or exposure to a pathogen as described herein. In some embodiments, a subject is suspected to have been exposed to a pathogen, e.g., a virus. In some embodiments, a subject has been exposed to an antigen or a protein representative or cross-reacts with antigens of a particular pathogen, e.g., a virus. In some embodiments, a subject has one or more symptoms that are indicative of a disease or condition associated with infection of or exposure to a pathogen as described herein. In some embodiments, the subject is currently infected by a pathogen, e.g., a virus described herein. In some embodiments, the subject is previously infected by a pathogen described herein. In some embodiments, a subject is a carrier of a virus described herein. In some embodiments, a subject is a carrier of fragments or remnants of a virus described herein. In some instances, a subject is carrier of adaptive immunity stemmed from previously or currently being infected by a virus described herein. In some embodiments, a subject is a carrier of adaptive immunity stemmed from previous or current exposure to a different virus or pathogen other than a virus or pathogen of interest.

The term “subject” can encompass mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

As used herein, the phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A. B, and C together.

The term “administering” and its grammatical equivalents as used herein can refer to providing one or more pharmaceutical compositions described herein to a subject, a patient, or a sample. By way of example and without limitation, “administering” can be performed by intravenous (i.v.) injection, sub-cutaneous (s.c.) injection, intradermal (i.d.) injection, intraperitoneal (i.p.) injection, intramuscular (i.m.) injection, intravascular injection, infusion (inf.), oral routes (p.o.), topical (top.) administration, or rectal (p.r.) administration. One or more such routes can be employed. Parenteral administration can be, for example, by bolus injection or by gradual perfusion over time.

The terms “treat.” “treating,” or “treatment,” and grammatical equivalents as used herein, can include alleviating, abating, or ameliorating at least one symptom of a disease or a condition, preventing additional symptoms, inhibiting the disease or the condition, e.g., arresting the development of the disease or the condition, relieving the disease or the condition, causing regression of the disease or the condition, relieving a condition caused by the disease or the condition, or stopping the symptoms of the disease or the condition either prophylactically and/or therapeutically.

### Types of Modifications

Modifications to sugars can alter physical property that influences oligonucleotide (e.g., gRNA, sgRNA) binding affinity for complementary strands, duplex formation, and/or interaction with nucleases. In some embodiments, a gRNA provided herein comprises one or more modifications with 2′-O-methyl (2′-O-Me), 2′-fluoro (2′-F), 2′-deoxy (2′-H), 2′-O-(2-methoxyethyl) (2′-MOE), 2′-NH2, 2′-arabinosyl (2′-arabino), 2′-F-arabinosyl (2′-F′arabino), 2′-O-Allyl, 2′-O-Ethylamine, 2′-O-Cyanoethyl, 2′-O-Acetalester, or a bicyclic nucleotide such as locked nucleic acid (LNA), 2′-unlocked nucleic acid (ULNA), a sugar in L form (L-sugar), 4′-thiribosyl, 2′-(5-constrained ethyl (S-cEt)), constrained MOE, 2′-0,4′-C-aminomethylene bridged nucleic acid (2′,4′-BNANC), or any combination thereof.

In some embodiments, a gRNA disclosed herein has 2′-O-methyl (2′O-Me) modifications that can increase binding affinity and/or nuclease stability (e.g., resistance to digestion/degradation by a nuclease, e.g., endonuclease or exonuclease) of oligonucleotides. The terms “mA,” “mC,” “mU,” or “mG” can be used to represent a nucleotide that has been modified with 2′O-Me.

In some embodiments, the gRNA has 2′-fluoro (2′-F) modifications on nucleotide sugar rings that can increase oligonucleotide binding affinity and/or nuclease stability. The terms “fA,” “fC,” “fU,” or “fG” can be used to represent a nucleotide that has been modified with 2′-F.

In some embodiments, the phosphate group of the gRNA is chemically modified. Examples of chemical modifications to the phosphate group include, but are not limited to, a phosphorothioate (PS), phosphonoacetate (PACE), thiophosphonoacetate (thioPACE), amide, triazole, phosphonate, or phosphotriester modification.

Phosphorothioate (PS) linkage or bond refers to a bond where a sulfur is substituted for one nonbridging phosphate oxygen in a phosphodiester linkage (e.g., bond between nucleotide bases). In some embodiments, the phosphorothioate linkage is used to generate modified gRNA. In some embodiments, modified gRNA with phosphorothioate linkage is referred to as S-oligos.

A “*” used herein can depict a PS modification. In some embodiments, the terms A*, C*, U*, or G* can denote a nucleotide that is linked to the next (e.g., 3′) nucleotide with a PS linkage. As described herein, the terms “mA*,” “mC*,” “mU*,” or “mG*” are used to denote a nucleotide with 2′-O-Me modification and linked to the next (e.g., 3′) nucleotide with a PS linkage.

In some embodiments, the nucleobase is chemically modified. Examples of chemical modifications to the nucleobase include, but are not limited to, 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, or halogenated aromatic groups.

In some embodiments, the gRNA is modified with sequence substitutions that do not comprise chemical modifications. In some embodiments, gRNA is engineered with G-C pairings. In some embodiments, gRNA is engineered with G-C pairings in the lower stem region. In some embodiments, gRNA is engineered with G-C pairings in the upper stem region.

Abasic nucleotides refer to nucleotides that lack nitrogenous bases. In some embodiments, the guide RNA (gRNA) has an abasic site (e.g., apurinic) that lacks a base. Inverted bases refer to bases with linkages that are inverted from the normal 5′ to 3′ linkage (e.g., 5′ to 5′ linkage, 3′ to 3′ linkage). In some embodiments, an abasic nucleotide is attached to the terminal 5′ nucleotide via 5′ to 5′ linkage. In some embodiments, an abasic nucleotide is attached to the terminal 3′ nucleotide via a 3′ to 3′ linkage.

### Guide RNA Compositions

Disclosed herein are compositions comprising a gRNA comprising a crRNA and tracrRNA that direct a nuclease such as Cas9 to a target DNA sequence. In some embodiments, the gRNA disclosed herein is associated on one RNA molecule. In some embodiments, the gRNA disclosed herein is single guide RNA (sgRNA).

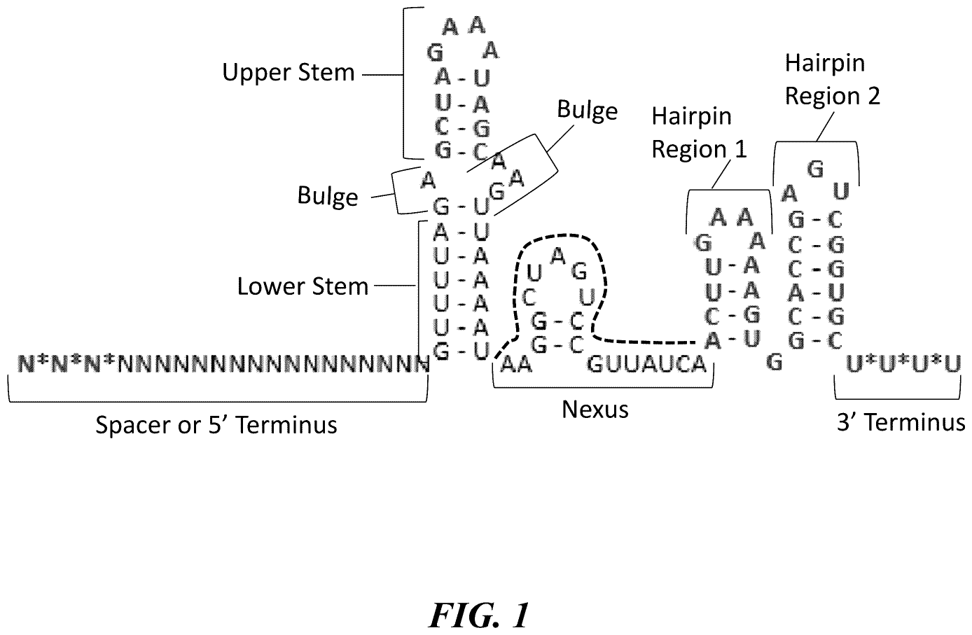

Briner A E et al., Molecular Cell 56:333-339 (2014) describes functional domains of sgRNAs, referred to herein as “regions”, including the “spacer” region (“5′ terminus”) responsible for targeting, the “lower stem”, the “bulge”, “upper stem” (which may include a tetraloop), the “nexus”, and the “hairpin 1” and “hairpin 2” regions. FIG. 1 illustrates exemplary domains of a sgRNA.

### 5′ Terminus Region

In some embodiments, sgRNA disclosed herein comprises nucleotides at the 5′ terminus region. The 5′ terminus region is sometimes referred to as the spacer. In some embodiments, the sgRNA does not comprise a 5′ terminus region. In some embodiments, the 5′ terminus of the sgRNA comprises a spacer or guide region that functions to direct a Cas protein to a target nucleotide sequence. In some embodiments, the 5′ terminus does not comprise a spacer or guide region. In some embodiments, the 5′ terminus comprises a spacer and additional nucleotides that do not function to direct a Cas protein to a target nucleotide region.

In some embodiments, the 5′ terminus comprises the first 1-10, 11, 12, 13, 14, 15, 16, 17, 18, 19, or 20 nucleotides at the 5′ end of the sgRNA. In some embodiments, the 5′ terminus comprises 20 nucleotides. In some embodiments, the 5′ terminus region comprises 5, 6, 7, 8, 9, 10, 11, 12, 13, 14, 15, 16, 17, 18, 19, 20, 21, 22, 23, 24, or 25 or more nucleotides. In some embodiments, the 5′ terminus comprises 17 nucleotides. In some embodiments, the 5′ terminus may comprise 18 nucleotides. In some embodiments, the 5′ terminus comprises 19 nucleotides.

The term “target sequence” or “target polynucleotide sequence” refers to a nucleic acid sequence present in a gene of interest. The target sequence can be in a genome of, or expressed in, a cell. In some embodiments, the target sequence is an exogenous sequence. In some embodiments, the target sequence in the gene of interest is complementary to the 5′ terminus of the sgRNA. In some embodiments, the degree of complementarity or identity between a 5′ terminus of a sgRNA and its corresponding target sequence in the gene of interest is about 50%, 55%, 60%, 65%, 70%, 75%, 80%, 85%, 90%, 95%, 96%, 97%, 98%, 99%, or 100%. In some embodiments, the 5′ terminus of a sgRNA and the target region of a gene of interest is 100% complementary or identical. In other embodiments, the 5′ terminus of a sgRNA and the target region of a gene of interest contains at least one mismatch. For example, the 5′ terminus of a sgRNA and the target sequence of a gene of interest can contain 1, 2, 3, 4, 5, 6, 7, 8, 9, or 10 mismatches, where the total length of the target sequence is at least about 17, 18, 19, 20 or more base pairs. In some embodiments, the 5′ terminus of a sgRNA and the target region of a gene of interest contains 1-6 mismatches where the guide sequence comprises at least about 17, 18, 19, 20 or more nucleotides. In some embodiments, the 5′ terminus of a sgRNA and the target region of a gene of interest contains 1, 2, 3, 4, 5, or 6 mismatches where the guide sequence comprises about 20 nucleotides. The 5′ terminus comprises nucleotides that are not considered guide regions (i.e., do not function to direct a cas9 protein to a target nucleic acid).

### Lower Stem

In some embodiments, the sgRNA comprises a lower stem region that is located between the 5′ terminus and the bulge region. In some embodiments, the lower stem region is separated from the upper stem region by a bulge. In some embodiments, the lower stem region comprises at least 1, at least 2, at least 3, at least 4, at least 5, at least 6, at least 7, at least 8, at least 9, at least 10, at least 11, at least 12, at least 13, at least 14, at least 15 or more nucleotides. In some embodiments, the lower stem region comprises at most 10, at most 11, at most 12, at most 13 at most 14, at most 15, at most 20, at most 25, at most 30, at most 35 or more nucleotides. In some embodiments, the lower stem region comprises 1, 2, 3, 4, 5, 6, 7, 8, 9, 10, 11, or 12 nucleotides.

In some embodiments, the lower stem region comprises 12 nucleotides. In some embodiments, the lower stem region has nucleotides that are complementary in nucleic acid sequence when read in opposite directions. In some embodiments, the complementarity in nucleic acid sequence of the lower stem leads to a secondary structure of a stem in the sgRNA (e.g., the regions may base pair with one another). In some embodiments, the lower stem region is not perfectly complementary to each other when read in opposite directions.

### Bulge

In some embodiments, the sgRNA comprises a bulge region that is located between the lower stem region and the upper stem region. In some embodiments, the bulge region comprises at least 1, at least 2, at least 3, at least 4, at least 5, at least 6, at least 7, at least 8, at least 9, at least 10, or more nucleotides. In some embodiments, the bulge region comprises at most 6, at most 7, at most 8, at most 9, at most 10, at most 15, at most 20, at most 25 or more nucleotides. In some embodiments, the bulge region comprises 1, 2, 3, 4, 5, or 6 nucleotides. In some embodiments, the bulge region comprises six nucleotides.

### Upper Sem

In some embodiments, the sgRNA comprises an upper stem region. In some embodiments, the upper stem region is separated from the upper stem region by a bulge. In some embodiments, the upper stem region comprises at least 1, at least 2, at least 3, at least 4, at least 5, at least 6, at least 7, at least 8, at least 9, at least 10, at least 11, at least 12, at least 13, at least 14, at least 15, or more nucleotides. In some embodiments, the upper stem region comprises at most 10, at most 11, at most 12, at most 13 at most 14, at most 15, at most 20, at most 25, at most 30, at most 35 or more nucleotides. In some embodiments, the upper stem region comprises 1, 2, 3, 4, 5, 6, 7, 8, 9, 10, 11, or 12 nucleotides. In some embodiments, the upper stem region comprises 12 nucleotides. In some embodiments, the upper stem region has nucleotides that are complementary in nucleic acid sequence when read in opposite directions. In some embodiments, the complementarity in nucleic acid sequence of upper stem leads to a secondary structure of a stem in the sgRNA (e.g., the regions may base pair with one another). In some embodiments, the upper stem region is not perfectly complementary to each other when read in opposite directions.

### Nexus

In some embodiments, the sgRNA comprises a nexus region that is located between the lower stem region and the hairpin region 1. In some embodiments, the upper stem region comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more nucleotides. In some embodiments, the upper stem region comprises at most 10, at most 11, at most 12, at most 13 at most 14, at most 15, at most 16, at most 17, at most 18, at most 19, at most 20, at most 25, at most 30, at most 35 or more nucleotides. In some embodiments, the upper stem region comprises 1, 2, 3, 4, 5, 6, 7, 8, 9, 10, 11, 12, 13, 14, 15, 16, 17, or 18 nucleotides. In some embodiments, the nexus comprises 18 nucleotides.

### Hairpin

In some embodiments, the sgRNA comprises one or more hairpin regions. In some embodiments, the hairpin region is downstream of (e.g., 3′ to) the nexus region. In some embodiments, the region of nucleotides immediately downstream of the nexus region is termed “hairpin region 1” or “H1”. In some embodiments, the region of nucleotides 3′ to hairpin 1 is termed “hairpin region 2” or “H2”. In some embodiments, the hairpin region comprises hairpin region 1 and hairpin region 2. In some embodiments, the sgRNA comprises only hairpin region 1 or hairpin region 2.

In some embodiments, the hairpin region 1 comprises 12 nucleic acids immediately downstream of the nexus region. In some embodiments, the hairpin 2 region comprises 15 nucleic acids downstream of the hairpin region 1.

In some embodiments, one or more nucleotides are present between the hairpin region 1 and the hairpin region 2. The one or more nucleotides between the hairpin region 1 and hairpin region 2 can be modified or unmodified. In some embodiments, hairpin region 1 and hairpin region 2 are separated by one nucleotide.

In some embodiments, a hairpin region has nucleotides that are complementary in nucleic acid sequence when read in opposite directions. In some embodiments, the hairpin regions are not perfectly complementary to each other when read in opposite directions (e.g., the top or loop of the hairpin comprises unpaired nucleotides).

In some embodiments, the hairpin region 1 of a sgRNA is replaced by 2 nucleotides.

### 3′ Terminus Region

In some embodiments, the sgRNA comprises nucleotides after the hairpin region(s), also called the 3′ terminus region. In some embodiments, the 3′ terminus region comprises 1, 2, 3, 4, 5, 6, 7, 8, 9, 10, 15, or 20 or more nucleotides, e.g. that are not associated with the secondary structure of a hairpin. In some embodiments, the 3′ terminus region comprises 1, 2, 3, or 4 nucleotides that are not associated with the secondary structure of a hairpin. In some embodiments, the 3′ terminus region comprises 4 nucleotides that are not associated with the secondary structure of a hairpin. In some embodiments, the 3′ terminus region comprises 1, 2, or 3 nucleotides that are not associated with the secondary structure of a hairpin.

### Tracr

Some exemplary tracr sequences are disclosed herein and other suitable sequences will be apparent to the skilled artisan based on the present disclosure and the knowledge in the art. Exemplary additional suitable tracr sequences include, without limitation, those recited in U.S. Pat. No. 11,479,767, the entire contents of which are incorporated herein by reference.

Modifications of Single Guide RNA (sgRNA)

Disclosed herein is a sgRNA comprising one or more modifications within one or more of the following regions: the nucleotides at the 5′ terminus; the lower stem region; the bulge region; the upper stem region; the nexus region; the hairpin region 1; the hairpin region 2; and the nucleotides at the 3′ terminus.

In some embodiments, the sgRNA comprises nucleotides that are not modified. In some embodiments, the sgRNA comprises no nucleotides that are not modified. In some embodiments, the sgRNA comprises one or more modifications with 2′-O-methyl (2′-O-Me), 2′-fluoro (2′-F), 2′-deoxy (2′-H), 2′-O-(2-methoxyethyl) (2′-MOE), 2′-NH2, 2′-arabinosyl (2′-arabino), 2′-F-arabinosyl (2′-F′arabino), 2′-O-Allyl, 2′-O-Ethylamine, 2′-O-Cyanoethyl, 2′-O-Acetalester, or a bicyclic nucleotide such as locked nucleic acid (LNA), 2′-unlocked nucleic acid (ULNA), a sugar in L form (L-sugar), 4′-thiribosyl, 2′-(5-constrained ethyl (S-cEt)), constrained MOE, 2′-0,4′-C-aminomethylene bridged nucleic acid (2′,4′-BNANC), or any combination thereof. In some embodiments, the sgRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 65, 70, 75, 80, 85, 90, 95, 100, 110, 120, 130, 140, 150 or more modified nucleotides. In some embodiments, the sgRNA comprises nucleotides that are all modified. In some embodiments, the sgRNA comprises a combination of different modified nucleotides. In some embodiments, the sgRNA comprises three or more modified nucleotides. In some embodiments, every other nucleotide of the sgRNA is modified. In some embodiments, the nucleotides in the 5′ half of the sgRNA are modified. In some embodiments, the nucleotides in the 3′ half of the sgRNA are modified. In some embodiments, the sgRNA comprises modified nucleotides that are arranged in contiguous stretch. In some embodiments, the sgRNA comprises at least one continuous stretch of modified nucleotides. In some embodiments, the sgRNA comprises a contiguous stretch of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more modified nucleotides. In some embodiments, the nucleotides of the sgRNA independently comprise one or more types of modifications. In some embodiments, the sgRNA comprises no modified nucleotides that are contiguous in the sequence of the sgRNA. In some embodiments, the sgRNA comprises some nucleotides that are contiguous in the sequence of the sgRNA.

In some embodiments, 2′-O-Me modification increases binding affinity of the sgRNA. In some embodiments, 2′-O-Me modification enhances nuclease stability of the sgRNA. In some embodiments, 2′-F modification increases the sgRNA binding affinity and nuclease stability.

In some embodiments, the 5′ terminus and/or 3′ terminus of the sgRNA comprises nucleotides that are not modified. In some embodiments, the lower stem region of the sgRNA comprises no nucleotides that are modified. In some embodiments, the 5′ terminus and/or 3′ terminus of the sgRNA disclosed herein comprises nucleotides that are modified. In some embodiments, the 5′ terminus and/or 3′ terminus of the sgRNA disclosed herein comprises nucleotides modified with 2′-O-methyl (2′-O-Me), 2′-fluoro (2′-F), 2′-deoxy (2′-H), 2′-O-(2-methoxyethyl) (2′-MOE), 2′-NH2, 2′-arabinosyl (2′-arabino), 2′-F-arabinosyl (2′-F′arabino), 2′-O-Allyl, 2′-O-Ethylamine, 2′-O-Cyanoethyl, 2′-O-Acetalester, or a bicyclic nucleotide such as locked nucleic acid (LNA), 2′-unlocked nucleic acid (ULNA), a sugar in L form (L-sugar), 4′-thiribosyl, 2′-(5-constrained ethyl (S-cEt)), constrained MOE, 2′-0,4′-C-aminomethylene bridged nucleic acid (2′,4′-BNANC), or any combination thereof. In some embodiments, the 5′ terminus and/or 3′ terminus comprises nucleotides that are all modified. In some embodiments, all the modifications of the 5′ terminus and/or 3′ terminus are the same. In some embodiments, the 5′ terminus and/or 3′ terminus comprises a combination of different modified nucleotides. In some embodiments, the 5′ terminus and/or 3′ terminus comprises three or more modified nucleotides. In some embodiments, every other nucleotide is modified in the 5′ terminus and/or 3′ terminus. In some embodiments, the nucleotides in the 5′ half of the 5′ terminus and/or 3′ terminus are modified. In some embodiments, the nucleotides in the 3′ half of the 5′ terminus and/or 3′ terminus are modified. In some embodiments, the 5′ terminus and/or 3′ terminus comprises modified nucleotides that are arranged in contiguous stretch. In some embodiments, the 5′ terminus and/or 3′ terminus comprises at least one continuous stretch of modified nucleotides. In some embodiments, the 5′ terminus and/or 3′ terminus comprises a contiguous stretch of at least 2, 3, 4, 5, 6, 7, 8, 9, 10, 11, 12, 13, 14, 15, 16, 17, 18, 19, 20, 21, 22, 23, 24, 25 or more modified nucleotides. In some embodiments, the nucleotides of the 5′ terminus and/or 3′ terminus independently comprise one or more types of modifications. In some embodiments, the lower stem region comprises no modified nucleotides that are contiguous in the sequence of the 5′ terminus and/or 3′ terminus. In some embodiments, the 5′ terminus and/or 3′ terminus comprises some modified nucleotides that are contiguous in the sequence of the gRNA. In some embodiments, the 5′ terminus and/or 3′ terminus comprises nucleotides modified with 2′-O-Me.

In some embodiments, the 5′ terminus comprises modifications at 1, 2, 3, or 4 of the first 4 nucleotides at its 5′ end. In some embodiments, the first three or four nucleotides at the 5′ terminus, and the last three or four nucleotides at the 3′ terminus are modified. In some embodiments, the first three nucleotides at the 5′ end of the 5′ terminus is modified. In some embodiments, the last three nucleotides at the 3′ end of the 3′ terminus is modified. In some embodiments, the first three nucleotides at the 5′ end of the 5′ terminus are modified with 2′-O-Me. In some embodiments, the last three nucleotides at the 3′ end of the 3′ terminus are modified with 2′-O-Me. In some embodiments, the nucleotides of the 5′ terminus other than the first 1, 2, 3, 4, 5, 6, 7, 8, 9, or 10 nucleotides are not modified.

In some embodiments, the phosphate group of the sgRNA is chemically modified. Examples of chemical modifications to the phosphate group includes, but are not limited to, a phosphorothioate (PS), phosphonoacetate (PACE), thiophosphonoacetate (thioPACE), amide, triazole, phosphonate, methylphophonoate-P(CH3), boranophosphonate, phosphorodithioate, or phosphotriester modification. In some embodiments, a 5′ end of the 5′ terminus and/or at a 3′ end of the 3′ terminus comprises one or more linkage modifications. In some embodiments, the linkage modification comprises phosphorothioate, phosphonoacetate, thiophosphonoacetate, methylphosphonoate-P(CH3), boranophosphonate, phosphorodithioate, or any combination thereof. In some embodiments, PS linkage refers to a bond where a sulfur is substituted for one nonbridging phosphate oxygen in a phosphodiester linkage, e.g., between nucleotides.

In some embodiments, the sgRNA comprises a phosphorothioate (PS) linkage at a 5′ end of the 5′ terminus or at a 3′ end of the 3′ terminus. In some embodiments, the sgRNA comprises a PS linkage at a 5′ end of the 5′ terminus. In some embodiments, the sgRNA comprises a PS linkage at a 3′ end of the 3′ terminus. In some embodiments, the sgRNA comprises a PS linkage at a 5′ end of the 5′ terminus and at a 3′ end of the 3′ terminus. In some embodiments, the sgRNA comprises one, two, or three, or more than three PS linkages at the 5′ end of the 5′ terminus or at the 3′ end of the 3′ terminus. In some embodiments, the sgRNA comprises three PS linkages at the 5′ end of the 5′ terminus or at the 3′ end of the 3′ terminus. In some embodiments, the sgRNA comprises three PS linkages at the 3′ end of the 3′ terminus. In some embodiments, the sgRNA comprises two and no more than two (i.e., only two) contiguous PS linkages at the 5′ end of the 5′ terminus or at the 3′ end of the 3′ terminus. In some embodiments, the sgRNA comprises three contiguous PS linkages at the 5′ end of the 5′ terminus or at the 3′ end of the 3′ terminus. In some embodiments, the 5′ end of the 5′ terminus comprises a PS linkage between the first and second nucleotides, and wherein the 5′ end does not comprise a PS linkage linking any two neighboring nucleotides form the second nucleotide to the fourth nucleotide. In some embodiments, the sgRNA comprises three PS linkages linking the first four nucleotides at the 5′ terminus and three PS bonds linking the last four nucleotides at the 3′ terminus. In some embodiments, the sgRNA further comprises 2′-O-Me modified nucleotides at the first three nucleotides at the 5′ terminus, and 2′-O-Me modified nucleic acids at the last four nucleotides at the 3′ terminus.

In some embodiments, the lower stem region of the sgRNA comprises nucleotides that are not modified. In some embodiments, the lower stem region of the sgRNA comprises no nucleotides that are modified. In some embodiments, the lower stem region of the sgRNA disclosed herein comprises nucleotides that are modified. In some embodiments, the lower stem region of the sgRNA disclosed herein comprises nucleotides modified with 2′-O-methyl (2′-O-Me), 2′-fluoro (2′-F), 2′-deoxy (2′-H), 2′-O-(2-methoxyethyl) (2′-MOE), 2′-NH2, 2′-arabinosyl (2′-arabino), 2′-F-arabinosyl (2′-F′arabino), 2′-O-Allyl, 2′-O-Ethylamine, 2′-O-Cyanoethyl, 2-O-Acetalester, or a bicyclic nucleotide such as locked nucleic acid (LNA), 2′-unlocked nucleic acid (ULNA), a sugar in L form (L-sugar), 4′-thiribosyl, 2′-(5-constrained ethyl (S-cEt)), constrained MOE, 2′-0,4′-C-aminomethylene bridged nucleic acid (2′,4′-BNANC), or any combination thereof. In some embodiments, the lower stem region comprises nucleotides that are all modified. In some embodiments, all the modifications of the lower stem region are the same. In some embodiments, the lower stem region comprises a combination of different modified nucleotides. In some embodiments, the lower stem region comprises three or more modified nucleotides. In some embodiments, every other nucleotide is modified in the lower stem region. In some embodiments, the nucleotides in the 5′ half of the lower stem region are modified. In some embodiments, the nucleotides in the 3′ half of the lower stem region are modified. In some embodiments, the lower stem region comprises modified nucleotides that are arranged in contiguous stretch. In some embodiments, the lower stem region comprises at least one continuous stretch of modified nucleotides. In some embodiments, the lower stem region comprises a contiguous stretch of at least 2, 3, 4, 5, 6, 7, 8, 9, 10, 11, 12, 13, 14, 15, 16, 17, 18, 19, 20, 21, 22, 23, 24, 25 or more modified nucleotides. In some embodiments, the nucleotides of the lower stem region independently comprises one or more types of modifications. In some embodiments, the lower stem region comprises no modified nucleotides that are contiguous in the sequence of the upper stem region. In some embodiments, the lower stem region comprises some modified nucleotides that are contiguous in the sequence of the gRNA. In some embodiments, the lower stem region comprises nucleotides modified with 2′-O-Me.

In some embodiments, the lower stem region comprises one or more linkage modifications. In some embodiments, the linkage modification comprises phosphorothioate (PS), phosphonoacetate, thiophosphonoacetate, methylphosphonoate-P(CH3), boranophosphonate, phosphorodithioate, or any combination thereof.

In some embodiments, the bulge region of the sgRNA comprises nucleotides that are not modified. In some embodiments, the bulge region of the sgRNA comprises no nucleotides that are modified. In some embodiments, the bulge region of the sgRNA comprises no nucleotides that are not modified. In some embodiments, the bulge region of the sgRNA disclosed herein comprises nucleotides that are modified. In some embodiments, the bulge region of the sgRNA disclosed herein comprises nucleotides modified with 2′-O-methyl (2′-O-Me), 2′-fluoro (2′-F), 2′-deoxy (2′-H), 2′-O-(2-methoxyethyl) (2′-MOE), 2′-NH2, 2′-arabinosyl (2′-arabino), 2′-F-arabinosyl (2′-F′arabino), 2′-O-Allyl, 2′-O-Ethylamine, 2′-O-Cyanoethyl, 2′-O-Acetalester, or a bicyclic nucleotide such as locked nucleic acid (LNA), 2′-unlocked nucleic acid (ULNA), a sugar in L form (L-sugar), 4′-thiribosyl, 2′-(5-constrained ethyl (S-cEt)), constrained MOE, 2′-0,4′-C-aminomethylene bridged nucleic acid (2′,4′-BNANC), or any combination thereof. In some embodiments, the bulge region comprises nucleotides that are all modified. In some embodiments, all the modifications of the bulge region are the same. In some embodiments, the bulge region comprises a combination of different modified nucleotides. In some embodiments, the bulge region comprises three or more modified nucleotides. In some embodiments, every other nucleotide is modified in the bulge region. In some embodiments, the nucleotides in the 5′ half of the bulge region are modified. In some embodiments, the nucleotides in the 3′ half of the bulge region are modified. In some embodiments, the bulge region comprises modified nucleotides that are arranged in contiguous stretch. In some embodiments, the upper stem region comprises at least one continuous stretch of modified nucleotides. In some embodiments, the bulge region comprises a contiguous stretch of at least 2, 3, 4, 5, 6, 7, 8, 9, 10, 11, 12, 13, 14, 15, 16, 17, 18, 19, 20, 21, 22, 23, 24, 25 or more modified nucleotides. In some embodiments, the nucleotides of the bulge region independently comprises one or more types of modifications. In some embodiments, the upper stem region comprises no modified nucleotides that are contiguous in the sequence of the upper stem region. In some embodiments, the bulge region comprises some modified nucleotides that are contiguous in the sequence of the gRNA. In some embodiments, the bulge region comprises nucleotides modified with 2′-O-Me.

In some embodiments, the bulge region comprises one or more linkage modifications. In some embodiments, the linkage modification comprises phosphorothioate (PS), phosphonoacetate, thiophosphonoacetate, methylphosphonoate-P(CH3), boranophosphonate, phosphorodithioate, or any combination thereof.

In some embodiments, the upper stem region of the sgRNA comprises nucleotides that are not modified. In some embodiments, the upper stem region of the sgRNA comprises no nucleotides that are modified. In some embodiments, the upper stem region of the sgRNA disclosed herein comprises nucleotides that are modified. In some embodiments, the upper stem region of the gRNA disclosed herein comprises nucleotides modified with 2′-O-methyl (2′-O-Me), 2′-fluoro (2′-F), 2′-deoxy (2′-H), 2′-O-(2-methoxyethyl) (2′-MOE), 2′-NH2, 2′-arabinosyl (2′-arabino), 2′-F-arabinosyl (2′-F′arabino), 2′-O-Allyl, 2′-O-Ethylamine, 2′-O-Cyanoethyl, 2′-O-Acetalester, or a bicyclic nucleotide such as locked nucleic acid (LNA), 2′-unlocked nucleic acid (ULNA), a sugar in L form (L-sugar), 4′-thiribosyl, 2′-(5-constrained ethyl (S-cEt)), constrained MOE, 2′-0,4′-C-aminomethylene bridged nucleic acid (2′,4′-BNANC), or any combination thereof. In some embodiments, the upper stem region comprises nucleotides that are all modified. In some embodiments, all the modifications of the upper stem region are the same. In some embodiments, the upper stem region comprises a combination of different modified nucleotides. In some embodiments, the upper stem region comprises three or more modified nucleotides. In some embodiments, every other nucleotide can be modified in the upper stem region. In some embodiments, the nucleotides in the 5′ half of the upper stem region are modified. In some embodiments, the nucleotides in the 3′ half of the upper stem region are modified. In some embodiments, the upper stem region comprises modified nucleotides that are arranged in contiguous stretch. In some embodiments, the upper stem region comprises at least one continuous stretch of modified nucleotides. In some embodiments, the upper stem region comprises a contiguous stretch of at least 2, 3, 4, 5, 6, 7, 8, 9, 10, 11, 12, 13, 14, 15, 16, 17, 18, 19, 20, 21, 22, 23, 24, 25 or more modified nucleotides. In some embodiments, the nucleotides of the upper stem region independently comprises one or more types of modifications. In some embodiments, the upper stem region comprises no modified nucleotides that are contiguous in the sequence of the upper stem region. In some embodiments, the upper stem region comprises some modified nucleotides that are contiguous in the sequence of the gRNA. In some embodiments, the upper stem region comprises nucleotides modified with 2′-O-Me.

In some embodiments, each nucleotide of the upper nucleotide is modified with 2′-O-Me except the first nucleotide of the upper stem region in the 5′ end to 3′ end direction. In some embodiments, each nucleotide of the upper nucleotide is modified with 2′-O-Me except the first nucleotide of the upper stem region in the 5′ end to 3′ end direction, and the first nucleotide of the upper stem region in the 5′ end to 3′ end direction is modified with a moiety other than 2′-O-Me, such as 2′-fluoro (2′-F), 2′-deoxy (2′-H), 2′-O-(2-methoxyethyl) (2′-MOE), 2′-NH2, 2′-arabinosyl (2′-arabino), 2′-F-arabinosyl (2′-F′arabino), 2′-O-Allyl, 2′-O-Ethylamine, 2′-O-Cyanoethyl, 2′-O-Acetalester, or a bicyclic nucleotide such as locked nucleic acid (LNA), 2′-unlocked nucleic acid (ULNA), a sugar in L form (L-sugar), 4′-thiribosyl, 2′-(5-constrained ethyl (S-cEt)), constrained MOE, 2′-0,4′-C-aminomethylene bridged nucleic acid (2′,4′-BNANC), or any combination thereof. In some embodiments, each nucleotide of the upper nucleotide is modified with 2′-O-Me except that the first nucleotide of the upper stem region in the 5′ end to 3′ end direction is not modified.

In some embodiments, each nucleotide of the upper nucleotide is modified with 2′-O-Me except the last nucleotide of the upper stem region in the 5′ end to 3′ end direction. In some embodiments, each nucleotide of the upper nucleotide is modified with 2′-O-Me except the last nucleotide of the upper stem region in the 5′ end to 3′ end direction, and the last nucleotide of the upper stem region in the 5′ end to 3′ end direction is modified with a moiety other than 2′-O-Me, such as 2′-fluoro (2′-F), 2′-deoxy (2′-H), 2′-O-(2-methoxyethyl) (2′-MOE), 2′-NH2, 2′-arabinosyl (2′-arabino), 2′-F-arabinosyl (2′-F′arabino), 2′-O-Allyl, 2′-O-Ethylamine, 2′-O-Cyanoethyl, 2′-O-Acetalester, or a bicyclic nucleotide such as locked nucleic acid (LNA), 2′-unlocked nucleic acid (ULNA), a sugar in L form (L-sugar), 4′-thiribosyl, 2′-(5-constrained ethyl (S-cEt)), constrained MOE, 2′-0,4′-C-aminomethylene bridged nucleic acid (2,4′-BNANC), or any combination thereof. In some embodiments, each nucleotide of the upper nucleotide is modified with 2′-O-Me except that the second nucleotide of the upper stem region in the 5′ end to 3′ end direction is not modified.

In some embodiments, each nucleotide of the upper nucleotide is modified with 2′-O-Me except the first and last nucleotide of the upper stem region in the 5′ end to 3′ end direction. In some embodiments, each nucleotide of the upper nucleotide is modified with 2′-O-Me except the first and the last nucleotides of the upper stem region in the 5′ end to 3′ end direction, and the first and the last nucleotides of the upper stem region in the 5′ end to 3′ end direction are independently modified with a moiety other than 2′-O-Me, such as 2′-fluoro (2′-F), 2′-deoxy (2′-H), 2′-O-(2-methoxyethyl) (2′-MOE), 2′-NH2, 2′-arabinosyl (2′-arabino), 2′-F-arabinosyl (2′-F′arabino), 2′-O-Allyl, 2′-O-Ethylamine, 2′-O-Cyanoethyl, 2′-O-Acetalester, or a bicyclic nucleotide such as locked nucleic acid (LNA), 2′-unlocked nucleic acid (ULNA), a sugar in L form (L-sugar), 4′-thiribosyl, 2′-(5-constrained ethyl (S-cEt)), constrained MOE, 2′-0,4′-C-aminomethylene bridged nucleic acid (2′,4′-BNANC), or any combination thereof. In some embodiments, each nucleotide of the upper nucleotide is modified with 2′-O-Me except that the first and the last nucleotides of the upper stem region in the 5′ end to 3′ end direction are not modified.

In some embodiments, the upper stem region comprises one or more linkage modifications. In some embodiments, the linkage modification comprises phosphorothioate (PS), phosphonoacetate, thiophosphonoacetate, methylphosphonoate-P(CH3), boranophosphonate, phosphorodithioate, or any combination thereof.

In some embodiments, the nexus region of the sgRNA comprises nucleotides that are not modified. In some embodiments, the nexus region of the sgRNA comprises no nucleotides that are modified. In some embodiments, the nexus region of the gRNA disclosed herein comprises nucleotides that are modified. In some embodiments, the nexus region of the sgRNA disclosed herein comprises nucleotides modified with 2′-O-methyl (2′-O-Me), 2′-fluoro (2′-F), 2′-deoxy (2′-H), 2′-O-(2-methoxyethyl) (2′-MOE), 2′-NH2, 2′-arabinosyl (2′-arabino), 2′-F-arabinosyl (2′-F′arabino), 2′-O-Allyl, 2′-O-Ethylamine, 2′-O-Cyanoethyl, 2′-O-Acetalester, or a bicyclic nucleotide such as locked nucleic acid (LNA), 2′-unlocked nucleic acid (ULNA), a sugar in L form (L-sugar), 4′-thioribosyl, 2′-(5-constrained ethyl (S-cEt)), constrained MOE, 2′-0,4′-C-aminomethylene bridged nucleic acid (2′,4′-BNANC), or any combination thereof. In some embodiments, the nexus region comprises nucleotides that are all modified. In some embodiments, all the modifications of the nexus region are the same. In some embodiments, the nexus region comprises a combination of different modified nucleotides. In some embodiments, the nexus region comprises three or more modified nucleotides. In some embodiments, every other nucleotide is modified in the nexus region. In some embodiments, the nucleotides in the 5′ half of the nexus region are modified. In some embodiments, the nucleotides in the 3′ half of the nexus region are modified. In some embodiments, the nexus region comprises modified nucleotides that are arranged in contiguous stretch. In some embodiments, the nexus region comprises at least one continuous stretch of modified nucleotides. In some embodiments, the nexus region comprises a contiguous stretch of at least 2, 3, 4, 5, 6, 7, 8, 9, 10, 11, 12, 13, 14, 15, 16, 17, 18, 19, 20, 21, 22, 23, 24, 25 or more modified nucleotides. In some embodiments, the nucleotides of the nexus region independently comprises one or more types of modifications. In some embodiments, the nexus region comprises no modified nucleotides that are contiguous in the sequence of the nexus region. In some embodiments, the nexus region comprises some modified nucleotides that are contiguous in the sequence of the gRNA. In some embodiments, the nexus region comprises nucleotides modified with 2′-O-Me.

In some embodiments, the nexus region comprises one or more linkage modifications. In some embodiments, the linkage modification comprises phosphorothioate (PS), phosphonoacetate, thiophosphonoacetate, methylphosphonoate-P(CH3), boranophosphonate, phosphorodithioate, or any combination thereof.

In some embodiments, the hairpin region 1 and/or hairpin region 2 of the sgRNA comprises nucleotides that are not modified. In some embodiments, the hairpin region 1 and/or hairpin region 2 of the sgRNA comprises no nucleotides that are modified. In some embodiments, the hairpin region 1 and/or hairpin region 2 of the sgRNA disclosed herein comprises nucleotides modified. In some embodiments, the hairpin region 1 and/or hairpin region 2 of the sgRNA disclosed herein comprises nucleotides modified with 2′-O-methyl (2′-O-Me), 2′-fluoro (2′-F), 2′-deoxy (2′-H), 2′-O-(2-methoxyethyl) (2′-MOE), 2′-NH2, 2′-arabinosyl (2′-arabino), 2′-F-arabinosyl (2′-F′arabino), 2′-O-Allyl, 2′-O-Ethylamine, 2′-O-Cyanoethyl, 2-O-Acetalester, or a bicyclic nucleotide such as locked nucleic acid (LNA), 2′-unlocked nucleic acid (ULNA), a sugar in L form (L-sugar), 4′-thiribosyl, 2′-(5-constrained ethyl (S-cEt)), constrained MOE, 2-0,4′-C-aninomethylene bridged nucleic acid (2′,4′-BNANC), or any combination thereof. In some embodiments, the hairpin region 1 and/or hairpin region 2 comprises nucleotides that are all modified. In some embodiments, all the modifications of the hairpin region 1 and/or hairpin region 2 are the same. In some embodiments, the hairpin region 1 and/or hairpin region 2 comprises a combination of different modified nucleotides. In some embodiments, the hairpin region 1 and/or hairpin region 2 comprises three or more modified nucleotides. In some embodiments, every other nucleotides is modified in the hairpin region 1 and/or hairpin region 2. In some embodiments, the nucleotides in the 5′ half of hairpin region 1 and/or hairpin region 2 are modified. In some embodiments, the nucleotides in the 3′ half of hairpin region 1 and/or hairpin region 2 are modified. In some embodiments, the hairpin region 1 and/or hairpin region 2 comprises modified nucleotides that are arranged in contiguous stretch. In some embodiments, the hairpin region 1 and/or hairpin region 2 comprises at least one continuous stretch of modified nucleotides. In some embodiments, the hairpin region 1 and/or hairpin region 2 comprises a contiguous stretch of at least 2, 3, 4, 5, 6, 7, 8, 9, 10, 11, 12, 13, 14, 15, 16, 17, 18, 19, 20, 21, 22, 23, 24, 25 or more modified nucleotides. In some embodiments, the nucleotides of the hairpin region 1 and/or hairpin region 2 independently comprises one or more types of modifications. In some embodiments, the hairpin region 1 and/or hairpin region 2 comprises no modified nucleotides that are contiguous in the sequence of the nexus region. In some embodiments, the hairpin region 1 and/or hairpin region 2 comprises some modified nucleotides that are contiguous in the sequence of the sgRNA. In some embodiments, the hairpin region 1 and/or hairpin region 2 comprises nucleotides modified with 2′-O-Me. In some embodiments, the hairpin region 2 comprises nucleotides modified with 2′-O-Me except the first nucleotide of the hairpin region 2. In some embodiments, the hairpin region 2 comprises nucleotides modified with 2′-O-Me, wherein the first nucleotide of the hairpin region 2 in a 5′ end to 3′ end direction is modified with a moiety other than 2′-O-Me, such as 2′-fluoro (2′-F), 2′-deoxy (2′-H), 2′-O-(2-methoxyethyl) (2′-MOE), 2′-NH2, 2′-arabinosyl (2-arabino), 2′-F-arabinosyl (2′-F′arabino), 2′-O-Allyl, 2′-O-Ethylamine, 2′-O-Cyanoethyl, 2′-O-Acetalester, or a bicyclic nucleotide such as locked nucleic acid (LNA), 2′-unlocked nucleic acid (ULNA), a sugar in L form (L-sugar), 4′-thiribosyl, 2′-(5-constrained ethyl (S-cEt)), constrained MOE, 2′-0,4′-C-aminomethylene bridged nucleic acid (2′,4′-BNANC), or any combination thereof. In some embodiments, the hairpin region 2 comprises nucleotides modified with 2′-O-Me, wherein the first nucleotide of the hairpin region 2 in a 5′ end to 3′ end direction is not modified.

In some embodiments, the hairpin region 1 and/or hairpin region 2 comprises one or more linkage modifications. In some embodiments, the linkage modification comprises phosphorothioate (PS), phosphonoacetate, thiophosphonoacetate, methylphosphonoate-P(CH3), boranophosphonate, phosphorodithioate, or any combination thereof.

In some embodiments, a sgRNA comprises an upper stem region, a hairpin region 1, a hairpin region 2, and a 5′ terminus, wherein the sgRNA comprise the following feature: (a) the upper stem region comprises nucleotides modified with 2′-O-Me, and wherein the first or last nucleotide of the upper stem region in a 5′ end to 3′ end direction is not modified with 2′O-Me; (b) the hairpin region 2 comprises nucleotides modified with 2′-O-Me except the first nucleotide of the hairpin region 2 in a 5′ end to 3′ end direction; (c) the 5′ terminus comprises a 5′ end modified with a phosphorothioate (PS) linkage between the first and second nucleotides, and wherein the 5′ end does not comprise a PS linkage linking any two neighboring nucleotides from the second nucleotide to the fourth nucleotide; or (d) any combination of (a)-(c).

In some embodiments, the sgRNA comprises an upper stem region comprising nucleotides modified with 2′-O-Me, and wherein the first nucleotide of the upper stem region in a 5′ end to 3′ end direction is not modified with 2′O-Me, and the hairpin region 2 comprising nucleotides modified with 2′-O-Me except the first nucleotide of the hairpin region 2 in a 5′ end to 3′ end direction. In some embodiments, the sgRNA comprises an upper stem region comprising nucleotides modified with 2′-O-Me, and wherein the last nucleotide of the upper stem region in a 5′ end to 3′ end direction is not modified with 2′O-Me, and the hairpin region 2 comprising nucleotides modified with 2′-O-Me except the first nucleotide of the hairpin region 2 in a 5′ end to 3′ end direction. In some embodiments, the sgRNA comprises a 5′ terminus comprising a 5′ end modified with a PS linkage between the first and second nucleotides, and wherein the 5′ end does not comprise a PS linkage linking any two neighboring nucleotides from the second nucleotide to the fourth nucleotide. In some embodiments, the sgRNA comprises the upper stem region comprising nucleotides modified with 2′-O-Me, and wherein the first or last nucleotide of the upper stem region in a 5′ end to 3′ end direction is not modified with 2′O-Me and the 5′ terminus comprises a 5′ end modified with a PS linkage between the first and second nucleotides, wherein the 5′ end does not comprise a PS linkage linking any two neighboring nucleotides from the second nucleotide to the fourth nucleotide. In some embodiments, the sgRNA comprises the hairpin region 2 comprising nucleotides modified with 2′-O-Me, and wherein the first nucleotide of the hairpin region 2 in a 5′ end to 3′ end direction is not modified and the 5′ terminus comprises a 5′ end modified with a PS linkage between the first and second nucleotides, wherein the 5′ end does not comprise a PS linkage linking any two neighboring nucleotides from the second nucleotide to the fourth nucleotide.

In some embodiments, the sgRNA described herein is unmodified 5′ terminus sequence of any one of the sequences set forth in SEQ ID NOs: 1, 2, 3, 4, or 5. A modification pattern includes the relative position and identity of modifications of the gRNA or a region of the sgRNA (e.g. 5′ terminus region, lower stem region, bulge region, upper stem region, nexus region, hairpin region 1, hairpin region 2, 3′ terminus region). In some embodiments, the modification pattern is at least 50%, at least 55%, at least 60%, at least 70%, at least 75%, at least 80%, at least 85%, at least 90%, at least 95%, at least 96%, at least 97%, at least 98%, and at least 99% of the modifications of any one of the modified sequences shown in the sequence column of Table 1, or over one or more regions of the sequence. In some embodiments, the modification pattern is at least 50%, at least 55%, at least 60%, at least 70%, at least 75%, at least 80%, at least 85%, at least 90%, at least 95%, at least 96%, at least 97%, at least 98%, and at least 99% identical over one or more regions of the sequence shown in Table 1, e.g., a 5′ terminus region, lower stem region, bulge region, upper stem region, nexus region, hairpin region 1, hairpin region 2, and/or 3′ terminus region. For example, in some embodiments, a gRNA is encompassed wherein the modification pattern is at least 50%, at least 55%, at least 60%, at least 70%, at least 75%, at least 80%, at least 85%, at least 90%, at least 95%, at least 96%, at least 97%, at least 98%, and at least 99% identical to the modification pattern of a sequence over the 5′ terminus region. In some embodiments, a gRNA is encompassed wherein the modification pattern is at least 50%, at least 55%, at least 60%, at least 70%, at least 75%, at least 80%, at least 85%, at least 90%, at least 95%, at least 9%%, at least 97%, at least 98%, and at least 99% identical over the lower stem. In some embodiments, a gRNA is encompassed wherein the modification pattern is at least 50%, at least 55%, at least 60%, at least 70%, at least 75%, at least 80%, at least 85%, at least 90%, at least 95%, at least 96%, at least 97%, at least 98%, and at least 99% identical over the bulge. In some embodiments, a gRNA is encompassed wherein the modification pattern is at least 50%, at least 55%, at least 60%, at least 70%, at least 75%, at least 80%, at least 85%, at least 90%, at least 95%, at least 96%, at least 97%, at least 98%, and at least 99% identical over the upper stem. In some embodiments, a gRNA is encompassed wherein the modification pattern is at least 50%, at least 55%, at least 60%, at least 70%, at least 75%, at least 80%, at least 85%, at least 90%, at least 95%, at least 96%, at least 97%, at least 98%, and at least 99% identical over the nexus. In some embodiments, a gRNA is encompassed wherein the modification pattern is at least 50%, at least 55%, at least 60%, at least 70%, at least 75%, at least 80%, at least 85%, at least 90%, at least 95%, at least 96%, at least 97%, at least 98%, and at least 99% identical over the hairpin region 1. In some embodiments, a gRNA is encompassed wherein the modification pattern is at least 50%, at least 55%, at least 60%, at least 70/o, at least 75%, at least 80%, at least 85%, at least 90%, at least 95%, at least 96%, at least 97%, at least 98%, and at least 99% identical over the hairpin region 2. In some embodiments, a gRNA is encompassed wherein the modification pattern is at least 50%, at least 55%, at least 60%, at least 70%, at least 75%, at least 80%, at least 85%, at least 90%, at least 95%, at least 96%, at least 97%, at least 98%, and at least 99% identical over the 3′ terminus. In some embodiments, the modification pattern differs from the modification pattern of a sequence of Table 1, or a region (e.g. 5′ terminus, lower stem, bulge, upper stem, nexus, hairpin region 1, hairpin region 2, 3′ terminus) of such a sequence, at 0, 1, 2, 3, 4, 5, 6 or more nucleotides. In some embodiments, the gRNA comprises modifications that differ from the modifications of a sequence of Table 1, at 0, 1, 2, 3, 4, 5, 6 or more nucleotides. In some embodiments, the gRNA comprises modifications that differ from modifications of a region (e.g. 5′ terminus, lower stem, bulge, upper stem, nexus, hairpin region 1, hairpin region 2, 3′ terminus) of a sequence of Table 1, at 0, 1, 2, 3, 4, 5, 6 or more nucleotides.

In some embodiments, the 5′ terminus of the sgRNA comprises at least about 75%, at least about 80%, at least about 81%, at least about 82%, at least about 83%, at least about 84%, at least about 85%, at least about 90%, at least about 91%, at least about 92%, at least about 93%, at least about 94%, at least about 95%, at least about 96%, at least about 97%, at least about 98%, at least about 99% or 100% sequence identity to any one of SEQ ID NOs: 1, 2, 3, 4, or 5. In some embodiments, the 5′ terminus of the sgRNA an comprise at least about 75%, at least about 80%, at least about 81%, at least about 82%, at least about 83%, at least about 84%, at least about 85%, at least about 90%, at least about 91%, at least about 92%, at least about 93%, at least about 94%, at least about 95%, at least about 96%, at least about 97%, at least about 98%, at least about 99% or 100% sequence identity to any one of SEQ ID NOs: 6-14.

In some embodiments, the TRACR region of the sgRNA an comprise at least about 75%, at least about 80%, at least about 81%, at least about 82%, at least about 83%, at least about 84%, at least about 85%, at least about 90%, at least about 91%, at least about 92%, at least about 93%, at least about 94%, at least about 95%, at least about 96%, at least about 97%, at least about 98%, at least about 99% or 100% sequence identity to any one of SEQ ID NOs: 15-17.

In some embodiments, the sgRNA comprises at least about 75%, at least about 80%, at least about 81%, at least about 82%, at least about 83%, at least about 84%, at least about 85%, at least about 90%, at least about 91%, at least about 92%, at least about 93%, at least about 94%, at least about 95%, at least about 96%, at least about 97%, at least about 98%, at least about 99% or 100% sequence identity to any one of SEQ ID NOs: 18-34.

As used in Table 1, “N” refers to any one of nucleotides A, G, C, and T. As used in Table 1, the ‘r’ refers to unmodified ribose, ‘m’ refers to ribose modified with 2′-O-Me, and ‘*’ refers to phosphorotioate linkage.

TABLE 1
Sequences

| | SEQ | |
|---|---|---|
| | ID | |
| Description | NO. | Sequence |
| Unmodified | 1 | ACUGCCUGGCUCACUCCUCC |
| SPACR/5′ | | |
| Terminus | | |
| gRNA 1 | | |
| Unmodified | 2 | UGCGGAAACCUUCUAGGGUG |
| SPACR/5′ | | |
| Terminus | | |
| gRNA 2 | | |
| Unmodified | 3 | AUCGUCCGAUGGGGCUCUGG |
| SPACR/5′ | | |
| Terminus | | |
| gRNA 3 | | |
| Unmodified | 4 | GCGGAAACCUUCUAGGGUGU |
| SPACR/5′ | | |
| Terminus | | |
| gRNA 4 | | |
| Unmodified | 5 | GUGUGGGUGCUUGACGCCUG |
| SPACR/5′ | | |
| terminus gRNA | | |
| 5 | | |
| Modified | 6 | mA*mC*mU*rGrCrCrUrGrGrCrUrCrArCrUrCrCrUrCrC |
| SPACR/5′ | | |
| Terminus | | |
| gRNA 1 | | |
| Modified | 7 | mU*mG*mC*rGrGrArArArCrCrUrUrCrUrArGrGrGrUrG |
| SPACR/5′ | | |
| Terminus | | |
| gRNA 2 | | |
| Modified | 8 | mA*mU*mC*rGrUrCrCrGrArUrGrGrGrGrCrUrCrUrGrG |
| SPACR/5′ | | |
| Terminus | | |
| gRNA 3 | | |
| Modified | 9 | mG*mC*mG*rGrArArArCrCrUrUrCrUrArGrGrGrUrGrU |
| SPACR/5′ | | |
| Terminus | | |
| gRNA 4 | | |
| Modified | 10 | mG*mU*mG*rUrGrGrGrUrGrCrUrUrGrArCrGrCrCrUrG |
| SPACR/5′ | | |
| Terminus | | |
| gRNA 5 | | |
| Modified | 11 | mA*mCmUrGrCrCrUrGrGrCrUrCrArCrUrCrCrUrCrC |
| SPACR/5′ | | |
| Terminus | | |
| gRNA 6 | | |
| Modified | 12 | mU*mGmCrGrGrArArArCrCrUrUrCrUrArGrGrGrUrG |
| SPACR/5′ | | |
| Terminus | | |
| gRNA 7 | | |
| Modified | 13 | mA*mUmCrGrUrCrCrGrArUrGrGrGrGrCrUrCrUrGrG |
| SPACR/5′ | | |
| terminus gRNA | | |
| 8 | | |
| Modified | 14 | mG*mCmGrGrArArArCrCrUrUrCrUrArGrGrGrUrGrU |
| SPACR/5′ | | |
| Terminus | | |
| gRNA 9 | | |
| Modified | 15 | rGrUrUrUrUrArGrAmGmCmUmAmGmAmAmAmUmAmGrCrArArG |
| TRACR | | rUrUrArArArArUrArArGrGrCrUrArGrUrCrCrGrUrUrArU |
| region 1 | | rCrAmAmCmUmUmGmAmAmAmAmAmGmUmGrGmCmAmCmCmGmAmG |
| | | mUmCmGmGmUmGmCmU*mU*mU*mU |
| Modified | 16 | rGrUrUrUrUrArGrArGmCmUmAmGmAmAmAmUmAmGmCrArArG |
| TRACR | | rUrUrArArArArUrArArGrGrCrUrArGrUrCrCrGrUrUrArU |
| region 2 | | rCrAmAmCmUmUmGmAmAmAmAmAmGmUmGrGmCmAmCmCmGmAmG |
| | | mUmCmGmGmUmGmCmU*mU*mU*mU |
| Modified | 17 | rGrUrUrUrUrArGrAmGmCmUmAmGmAmAmAmUmAmGmCrArArG |
| TRACR | | rUrUrArArArArUrArArGrGrCrUrArGrUrCrCrGrUrUrArU |
| region 3 | | rCrAmAmCmUmUmGmAmAmAmAmAmGmUmGmGmCmAmCmCmGmAmG |
| | | mUmCmGmGmUmGmCmU*mU*mU*mU |
| sgRNA 1 | 18 | mA*mC*mU*rGrCrCrUrGrGrCrUrCrArCrUrCrCrUrCrCrGr |
| | | UrUrUrUrArGrAmGmCmUmAmGmAmAmAmUmAmGrCrArArGrUr |
| | | UrArArArArUrArArGrGrCrUrArGrUrCrCrGrUrUrArUrCr |
| | | AmAmCmUmUmGmAmAmAmAmAmGmUmGrGmCmAmCmCmGmAmGmUm |
| | | CmGmGmUmGmCmU*mU*mU*mU |
| sgRNA 2 | 19 | mU*mG*mC*rGrGrArArArCrCrUrUrCrUrArGrGrGrUrGrGr |
| | | UrUrUrUrArGrAmGmCmUmAmGmAmAmAmUmAmGrCrArArGrUr |
| | | UrArArArArUrArArGrGrCrUrArGrUrCrCrGrUrUrArUrCr |
| | | AmAmCmUmUmGmAmAmAmAmAmGmUmGrGmCmAmCmCmGmAmGmUm |
| | | CmGmGmUmGmCmU*mU*mU*mU |
| sgRNA 3 | 20 | mA*mU*mC*rGrUrCrCrGrArUrGrGrGrGrCrUrCrUrGrGrGr |
| | | UrUrUrUrArGrAmGmCmUmAmGmAmAmAmUmAmGrCrArArGrUr |
| | | UrArArArArUrArArGrGrCrUrArGrUrCrCrGrUrUrArUrCr |
| | | AmAmCmUmUmGmAmAmAmAmAmGmUmGrGmCmAmCmCmGmAmGmUm |
| | | CmGmGmUmGmCmU*mU*mU*mU |
| sgRNA 4 | 21 | mG*mC*mG*rGrArArArCrCrUrUrCrUrArGrGrGrUrGrUrGr |
| | | UrUrUrUrArGrAmGmCmUmAmGmAmAmAmUmAmGrCrArArGrUr |
| | | UrArArArArUrArArGrGrCrUrArGrUrCrCrGrUrUrArUrCr |
| | | AmAmCmUmUmGmAmAmAmAmAmGmUmGrGmCmAmCmCmGmAmGmUm |
| | | CmGmGmUmGmCmU*mU*mU*mU |
| sgRNA 5 | 22 | mG*mU*mG*rUrGrGrGrUrGrCrUrUrGrArCrGrCrCrUrGrGr |
| | | UrUrUrUrArGrAmGmCmUmAmGmAmAmAmUmAmGrCrArArGrUr |
| | | UrArArArArUrArArGrGrCrUrArGrUrCrCrGrUrUrArUrCr |
| | | AmAmCmUmUmGmAmAmAmAmAmGmUmGrGmCmAmCmCmGmAmGmUm |
| | | CmGmGmUmGmCmU*mU*mU*mU |
| sgRNA 6 | 23 | mA*mC*mU*rGrCrCrUrGrGrCrUrCrArCrUrCrCrUrCrCrGr |
| | | UrUrUrUrArGrArGmCmUmAmGmAmAmAmUmAmGmCrArArGrUr |
| | | UrArArArArUrArArGrGrCrUrArGrUrCrCrGrUrUrArUrCr |
| | | AmAmCmUmUmGmAmAmAmAmAmGmUmGrGmCmAmCmCmGmAmGmUm |
| | | CmGmGmUmGmCmU*mU*mU*mU |
| sgRNA 7 | 24 | mU*mG*mC*rGrGrArArArCrCrUrUrCrUrArGrGrGrUrGrGr |
| | | UrUrUrUrArGrArGmCmUmAmGmAmAmAmUmAmGmCrArArGrUr |
| | | UrArArArArUrArArGrGrCrUrArGrUrCrCrGrUrUrArUrCr |
| | | AmAmCmUmUmGmAmAmAmAmAmGmUmGrGmCmAmCmCmGmAmGmUm |
| | | CmGmGmUmGmCmU*mU*mU*mU |
| sgRNA 8 | 25 | mA*mU*mC*rGrUrCrCrGrArUrGrGrGrGrCrUrCrUrGrGrGr |
| | | UrUrUrUrArGrArGmCmUmAmGmAmAmAmUmAmGmCrArArGrUr |
| | | UrArArArArUrArArGrGrCrUrArGrUrCrCrGrUrUrArUrCr |
| | | AmAmCmUmUmGmAmAmAmAmAmGmUmGrGmCmAmCmCmGmAmGmUm |
| | | CmGmGmUmGmCmU*mU*mU*mU |
| sgRNA 9 | 26 | mG*mC*mG*rGrArArArCrCrUrUrCrUrArGrGrGrUrGrUrGr |
| | | UrUrUrUrArGrArGmCmUmAmGmAmAmAmUmAmGmCrArArGrUr |
| | | UrArArArArUrArArGrGrCrUrArGrUrCrCrGrUrUrArUrCr |
| | | AmAmCmUmUmGmAmAmAmAmAmGmUmGrGmCmAmCmCmGmAmGmUm |
| | | CmGmGmUmGmCmU*mU*mU*mU |
| sgRNA 10 | 27 | mG*mU*mG*rUrGrGrGrUrGrCrUrUrGrArCrGrCrCrUrGrGr |
| | | UrUrUrUrArGrArGmCmUmAmGmAmAmAmUmAmGmCrArArGrUr |
| | | UrArArArArUrArArGrGrCrUrArGrUrCrCrGrUrUrArUrCr |
| | | AmAmCmUmUmGmAmAmAmAmAmGmUmGrGmCmAmCmCmGmAmGmUm |
| | | CmGmGmUmGmCmU*mU*mU*mU |
| sgRNA 11 | 28 | mA*mCmUrGrCrCrUrGrGrCrUrCrArCrUrCrCrUrCrCrGrUr |
| | | UrUrUrArGrAmGmCmUmAmGmAmAmAmUmAmGmCrArArGrUrUr |
| | | ArArArArUrArArGrGrCrUrArGrUrCrCrGrUrUrArUrCrAm |
| | | AmCmUmUmGmAmAmAmAmAmGmUmGmGmCmAmCmCmGmAmGmUmCm |
| | | GmGmUmGmCmU*mU*mU*mU |
| sgRNA 12 | 29 | mU*mGmCrGrGrArArArCrCrUrUrCrUrArGrGrGrUrGrGrUr |
| | | UrUrUrArGrAmGmCmUmAmGmAmAmAmUmAmGmCrArArGrUrUr |
| | | ArArArArUrArArGrGrCrUrArGrUrCrCrGrUrUrArUrCrAm |
| | | AmCmUmUmGmAmAmAmAmAmGmUmGmGmCmAmCmCmGmAmGmUmCm |
| | | GmGmUmGmCmU*mU*mU*mU |
| sgRNA 13 | 30 | mA*mUmCrGrUrCrCrGrArUrGrGrGrGrCrUrCrUrGrGrGrUr |
| | | UrUrUrArGrAmGmCmUmAmGmAmAmAmUmAmGmCrArArGrUrUr |
| | | ArArArArUrArArGrGrCrUrArGrUrCrCrGrUrUrArUrCrAm |
| | | AmCmUmUmGmAmAmAmAmAmGmUmGmGmCmAmCmCmGmAmGmUmCm |
| | | GmGmUmGmCmU*mU*mU*mU |
| sgRNA 14 | 31 | mG*mCmGrGrArArArCrCrUrUrCrUrArGrGrGrUrGrUrGrUr |
| | | UrUrUrArGrAmGmCmUmAmGmAmAmAmUmAmGmCrArArGrUrUr |
| | | ArArArArUrArArGrGrCrUrArGrUrCrCrGrUrUrArUrCrAm |
| | | AmCmUmUmGmAmAmAmAmAmGmUmGmGmCmAmCmCmGmAmGmUmCm |
| | | GmGmUmGmCmU*mU*mU*mU |
| mod 1 | 32 | mN*mN*mN*NNNNNNNNNNNNNNNNNrGrUrUrUrUrArGrAmGmC |
| | | mUmAmGmAmAmAmUmAmGrCrArArGrUrUrArArArArUrArArG |
| | | rGrCrUrArGrUrCrCrGrUrUrArUrCrAmAmCmUmUmGmAmAmA |
| | | mAmAmGmUmGrGmCmAmCmCmGmAmGmUmCmGmGmUmGmCmU*mU* |
| | | mU*mU |
| mod 2 | 33 | mN*mN*mN*NNNNNNNNNNNNNNNNNrGrUrUrUrUrArGrArGmC |
| | | mUmAmGmAmAmAmUmAmGmCrArArGrUrUrArArArArUrArArG |
| | | rGrCrUrArGrUrCrCrGrUrUrArUrCrAmAmCmUmUmGmAmAmA |
| | | mAmAmGmUmGrGmCmAmCmCmGmAmGmUmCmGmGmUmGmCmU*mU* |
| | | mU*mU |
| mod 3 | 34 | mN*mNmNNNNNNNNNNNNNNNNNNrGrUrUrUrUrArGrAmGmCmU |
| | | mAmGmAmAmAmUmAmGmCrArArGrUrUrArArArArUrArArGrG |
| | | rCrUrArGrUrCrCrGrUrUrArUrCrAmAmCmUmUmGmAmAmAmA |
| | | mAmGmUmGmGmCmAmCmCmGmAmGmUmCmGmGmUmGmCmU*mU*mU |
| | | *mU |
| Parental Guide | 35 | mA*mC*mU*rGrCrCrUrGrGrCrUrCrArCrUrCrCrUrCrCrGr |
| 1 | | UrUrUrUrArGrAmGmCmUmAmGmAmAmAmUmAmGmCrArArGrUr |
| | | UrArArArArUrArArGrGrCrUrArGrUrCrCrGrUrUrArUrCr |
| | | AmAmCmUmUmGmAmAmAmAmAmGmUmGmGmCmAmCmCmGmAmGmUm |
| | | CmGmGmUmGmCmU*mU*mU*mU |
| Parental Guide | 36 | mA*mU*mC*rGrUrCrCrGrArUrGrGrGrGrCrUrCrUrGrGrGr |
| 2 | | UrUrUrUrArGrAmGmCmUmAmGmAmAmAmUmAmGmCrArArGrUr |
| | | UrArArArArUrArArGrGrCrUrArGrUrCrCrGrUrUrArUrCr |
| | | AmAmCmUmUmGmAmAmAmAmAmGmUmGmGmCmAmCmCmGmAmGmUm |
| | | CmGmGmUmGmCmU*mU*mU*mU |
| Fusion Protein | 37 | MPKKKRKVPKKKRKVYNHDQEFDPPKVYPPVPAEKRKPIRVLSLFD |
| 1 | | GIATGLLVLKDLGIQVDRYIASEVCEDSITVGMVRHQGKIMYVGDV |
| | | RSVTQKHIQEWGPFDLVIGGSPCNDLSIVNPARKGLYEGTGRLFFE |
| | | FYRLLHDARPKEGDDRPFFWLFENVVAMGVSDKRDISRFLESNPVM |
| | | IDAKEVSAAHRARYFWGNLPGMNRPLASTVNDKLELQECLEHGRIA |
| | | KFSKVRTITTRSNSIKQGKDQHFPVFMNEKEDILWCTEMERVFGFP |
| | | VHYTDVSNMSRLARQRLLGRSWSVPVIRHLFAPLKEYFACVSSGNS |
| | | NANSRGPSFSSGLVPLSLRGSHMAAIPALDPEAEPSMDVILVGSSE |
| | | LSSSVSPGTGRDLIAYEVKANQRNIEDICICCGSLQVHTQHPLFEG |
| | | GICAPCKDKFLDALFLYDDDGYQSYCSICCSGETLLICGNPDCTRC |
| | | YCFECVDSLVGPGTSGKVHAMSNWVCYLCLPSSRSGLLQRRRKWRS |
| | | QLKAFYDRESENPLEMFETVPVWRRQPVRVLSLFEDIKKELTSLGF |
| | | LESGSDPGQLKHVVDVTDTVRKDVEEWGPFDLVYGATPPLGHTCDR |
| | | PPSWYLFQFHRLLQYARPKPGSPRPFFWMFVDNLVLNKEDLDVASR |
| | | FLEMEPVTIPDVHGGSLQNAVRVWSNIPAIRSRHWALVSEEELSLL |
| | | AQNKQSSKLAAKWPTKLVKNCFLPLREYFKYFSTELTSSLGGPSSG |
| | | APPPSGGSPAGSPTSTEEGTSESATPESGPGTSTEPSEGSAPGSPA |
| | | GSPTSTEEGTSTEPSEGSAPGTSTEPSELEDKKYSIGLAIGTNSVG |
| | | WAVITDEYKVPSKKFKVLGNTDRHSIKKNLIGALLFDSGETAEATR |
| | | LKRTARRRYTRRKNRICYLQEIFSNEMAKVDDSFFHRLEESFLVEE |
| | | DKKHERHPIFGNIVDEVAYHEKYPTIYHLRKKLVDSTDKADLRLIY |
| | | LALAHMIKFRGHFLIEGDLNPDNSDVDKLFIQLVQTYNQLFEENPI |
| | | NASGVDAKAILSARLSKSRRLENLIAQLPGEKKNGLFGNLIALSLG |
| | | LTPNFKSNFDLAEDAKLQLSKDTYDDDLDNLLAQIGDQYADLFLAA |
| | | KNLSDAILLSDILRVNTEITKAPLSASMIKRYDEHHQDLTLLKALV |
| | | RQQLPEKYKEIFFDQSKNGYAGYIDGGASQEEFYKFIKPILEKMDG |
| | | TEELLVKLNREDLLRKQRTFDNGSIPHQIHLGELHAILRRQEDFYP |
| | | FLKDNREKIEKILTFRIPYYVGPLARGNSRFAWMTRKSEETITPWN |
| | | FEEVVDKGASAQSFIERMTNFDKNLPNEKVLPKHSLLYEYFTVYNE |
| | | LTKVKYVTEGMRKPAFLSGEQKKAIVDLLFKTNRKVTVKQLKEDYF |
| | | KKIECFDSVEISGVEDRFNASLGTYHDLLKIIKDKDFLDNEENEDI |
| | | LEDIVLTLTLFEDREMIEERLKTYAHLFDDKVMKQLKRRRYTGWGR |
| | | LSRKLINGIRDKQSGKTILDFLKSDGFANRNFMQLIHDDSLTFKED |
| | | IQKAQVSGQGDSLHEHIANLAGSPAIKKGILQTVKVVDELVKVMGR |
| | | HKPENIVIEMARENQTTQKGQKNSRERMKRIEEGIKELGSQILKEH |
| | | PVENTQLQNEKLYLYYLQNGRDMYVDQELDINRLSDYDVDAIVPQS |
| | | FLKDDSIDNKVLTRSDKNRGKSDNVPSEEVVKKMKNYWRQLLNAKL |
| | | ITQRKFDNLTKAERGGLSELDKAGFIKRQLVETRQITKHVAQILDS |
| | | RMNTKYDENDKLIREVKVITLKSKLVSDFRKDFQFYKVREINNYHH |
| | | AHDAYLNAVVGTALI |
| Fusion Protein | 38 | KKYPKLESEFVYGDYKVYDVRKMIAKSEQEIGKATAKYFFYSNIMN |
| 1 plasmid | | FFKTEITLANGEIRKRPLIETNGETGEIVWDKGRDFATVRKVLSMP |
| sequence | | QVNIVKKTEVQTGGFSKESILPKRNSDKLIARKKDWDPKKYGGFDS |
| | | PTVAYSVLVVAKVEKGKSKKLKSVKELLGITIMERSSFEKNPIDFL |
| | | EAKGYKEVKKDLIIKLPKYSLFELENGRKRMLASAGELQKGNELAL |
| | | PSKYVNFLYLASHYEKLKGSPEDNEQKQLFVEQHKHYLDEIIEQIS |
| | | EFSKRVILADANLDKVLSAYNKHRDKPIREQAENIIHLFTLTNLGA |
| | | PAAFKYFDTTIDRKRYTSTKEVLDATLIHQSITGLYETRIDLSQLG |
| | | GDSPKKKRKVGVDGSSGSETPGTSESATPESTGMNNSQGRVTFEDV |
| | | TVNFTQGEWQRLNPEQRNLYRDVMLENYSNLVSVGQGETTKPDVIL |
| | | RLEQGKEPWLEEEEVLGSGRAEKNGDIGGQIWKPKDVKESLSAPKK |
| | | KRKVPKKKRKVGGGCGCTCGAGCAGGTTCAGAAGGAGATCAAAAAC |
| | | CCCCAAGGATCAAACATGCCAAAAAAGAAGAGAAAGGTACCGAAGA |
| | | AAAAAAGAAAGGTATACAATCACGATCAGGAGTTCGACCCCCCTAA |
| | | GGTGTACCCACCAGTGCCTGCAGAGAAGAGGAAGCCAATCCGGGTG |
| | | CTGAGCCTGTTTGATGGCATCGCCACCGGCCTGCTGGTGCTGAAGG |
| | | ATCTGGGCATCCAGGTGGACCGGTACATCGCCTCCGAGGTGTGCGA |
| | | GGATTCTATCACCGTGGGCATGGTGCGCCACCAGGGCAAGATCATG |
| | | TATGTGGGCGACGTGCGGTCCGTGACACAGAAGCACATCCAGGAGT |
| | | GGGGCCCATTCGATCTGGTGATCGGCGGCAGCCCCTGTAATGACCT |
| | | GTCCATCGTGAACCCTGCAAGGAAGGGACTGTACGAGGGAACCGGC |
| | | CGGCTGTTCTTTGAGTTTTATAGACTGCTGCACGACGCCAGGCCTA |
| | | AGGAGGGCGACGATAGACCATTCTTTTGGCTGTTCGAGAATGTGGT |
| | | GGCTATGGGCGTGAGCGATAAGAGGGACATCTCCAGGTTTCTGGAG |
| | | TCTAACCCCGTGATGATCGATGCAAAGGAGGTGTCCGCCGCACACA |
| | | GAGCCAGGTATTTCTGGGGCAATCTGCCAGGAATGAACAGGCCACT |
| | | GGCAAGCACCGTGAATGACAAGCTGGAGCTGCAGGAGTGCCTGGAG |
| | | CACGGAAGGATCGCCAAGTTTTCCAAGGTGCGCACAATCACCACAC |
| | | GGAGCAATTCCATCAAGCAGGGCAAGGATCAGCACTTCCCCGTGTT |
| | | CATGAACGAGAAGGAGGACATCCTGTGGTGTACCGAGATGGAGAGA |
| | | GTGTTCGGCTTTCCAGTGCACTACACAGACGTGTCTAACATGAGCA |
| | | GGCTGGCAAGGCAGCGGCTGCTGGGCAGATCTTGGAGCGTGCCCGT |
| | | GATCAGGCACCTGTTCGCCCCTCTGAAGGAGTATTTTGCCTGCGTG |
| | | AGCAGCGGCAACTCCAATGCCAACAGCCGGGGCCCCTCTTTCAGCT |
| | | CCGGATTGGTGCCTCTGAGCCTGAGGGGCTCCCACATGGCAGCAAT |
| | | CCCCGCCCTGGACCCCGAGGCCGAGCCTAGCATGGACGTGATCCTG |
| | | GTGGGCTCTAGCGAGCTGTCCTCTAGCGTGTCTCCAGGAACCGGAA |
| | | GGGATCTGATCGCATACGAGGTGAAGGCCAATCAGCGGAACATCGA |
| | | GGACATCTGTATCTGCTGTGGCAGCCTGCAGGTGCACACACAGCAC |
| | | CCACTGTTCGAGGGAGGAATCTGCGCACCCTGTAAGGATAAGTTCC |
| | | TGGACGCCCTGTTTCTGTACGACGATGACGGCTACCAGTCCTATTG |
| | | CTCTATCTGCTGTTCCGGCGAGACCCTGCTGATCTGCGGCAATCCA |
| | | GATTGTACAAGGTGCTATTGTTTTGAGTGCGTGGACTCTCTGGTGG |
| | | GACCAGGCACCAGCGGAAAGGTGCACGCCATGTCCAACTGGGTGTG |
| | | CTACCTGTGCCTGCCATCCTCTCGCAGCGGACTGCTGCAGCGGAGA |
| | | AGGAAGTGGAGATCCCAGCTGAAGGCCTTCTATGATAGGGAGTCTG |
| | | AGAACCCCCTGGAGATGTTTGAGACCGTGCCAGTGTGGCGCCGGCA |
| | | GCCCGTGAGGGTGCTGAGCCTGTTCGAGGATATCAAGAAGGAGCTG |
| | | ACATCCCTGGGCTTTCTGGAGTCCGGCTCTGACCCCGGACAGCTGA |
| | | AGCACGTGGTGGATGTGACCGACACAGTGCGGAAGGATGTGGAGGA |
| | | GTGGGGCCCTTTCGACCTGGTGTACGGAGCAACCCCTCCACTGGGA |
| | | CACACATGCGACAGACCCCCTTCTTGGTACCTGTTCCAGTTTCACC |
| | | GCCTGCTGCAGTATGCAAGGCCAAAGCCAGGCAGCCCTAGACCATT |
| | | CTTTTGGATGTTCGTGGATAATCTGGTGCTGAACAAGGAGGATCTG |
| | | GACGTGGCCAGCAGGTTTCTGGAGATGGAGCCAGTGACCATCCCAG |
| | | ACGTGCACGGCGGCTCCCTGCAGAATGCCGTGCGCGTGTGGTCTAA |
| | | CATCCCTGCCATCAGAAGCAGGCACTGGGCACTGGTGAGCGAGGAG |
| | | GAGCTGTCCCTGCTGGCCCAGAATAAGCAGAGCAGCAAGCTGGCCG |
| | | CCAAGTGGCCTACAAAGCTGGTGAAGAACTGCTTCCTGCCACTGCG |
| | | GGAGTACTTCAAGTATTTTTCCACCGAGCTGACATCTAGCCTGGGA |
| | | GGACCCTCCTCTGGCGCCCCACCACCTAGCGGCGGCTCCCCTGCCG |
| | | GCTCTCCAACCAGCACAGAGGAGGGCACCAGCGAGTCCGCCACACC |
| | | AGAGTCTGGACCTGGCACCAGCACAGAGCCATCCGAGGGCTCTGCC |
| | | CCAGGCTCTCCTGCAGGCAGCCCTACCTCCACCGAAGAGGGCACCA |
| | | GCACAGAGCCTTCTGAGGGCAGCGCCCCAGGCACCTCTACAGAGCC |
| | | AAGCGAGCTCGAGGACAAGAAGTACAGCATCGGCCTGGCCATCGGC |
| | | ACCAACTCTGTGGGCTGGGCCGTGATCACCGACGAGTACAAGGTGC |
| | | CCAGCAAGAAATTCAAGGTGCTGGGCAACACCGACCGGCACAGCAT |
| | | CAAGAAGAACCTGATCGGAGCCCTGCTGTTCGACAGCGGCGAAACA |
| | | GCCGAGGCCACCCGGCTGAAGAGAACCGCCAGAAGAAGATACACCA |
| | | GACGGAAGAACCGGATCTGCTATCTGCAAGAGATCTTCAGCAACGA |
| | | GATGGCCAAGGTGGACGACAGCTTCTTCCACAGACTGGAAGAGTCC |
| | | TTCCTGGTGGAAGAGGATAAGAAGCACGAGCGGCACCCCATCTTCG |
| | | GCAACATCGTGGACGAGGTGGCCTACCACGAGAAGTACCCCACCAT |
| | | CTACCACCTGAGAAAGAAACTGGTGGACAGCACCGACAAGGCCGAC |
| | | CTGCGGCTGATCTATCTGGCCCTGGCCCACATGATCAAGTTCCGGG |
| | | GCCACTTCCTGATCGAGGGCGACCTGAACCCCGACAACAGCGACGT |
| | | GGACAAGCTGTTCATCCAGCTGGTGCAGACCTACAACCAGCTGTTC |
| | | GAGGAAAACCCCATCAACGCCAGCGGCGTGGACGCCAAGGCCATCC |
| | | TGTCTGCCAGACTGAGCAAGAGCAGACGGCTGGAAAATCTGATCGC |
| | | CCAGCTGCCCGGCGAGAAGAAGAATGGCCTGTTCGGCAACCTGATT |
| | | GCCCTGAGCCTGGGCCTGACCCCCAACTTCAAGAGCAACTTCGACC |
| | | TGGCCGAGGATGCCAAACTGCAGCTGAGCAAGGACACCTACGACGA |
| | | CGACCTGGACAACCTGCTGGCCCAGATCGGCGACCAGTACGCCGAC |
| | | CTGTTTCTGGCCGCCAAGAACCTGTCCGACGCCATCCTGCTGAGCG |
| | | ACATCCTGAGAGTGAACACCGAGATCACCAAGGCCCCCCTGAGCGC |
| | | CTCTATGATCAAGAGATACGACGAGCACCACCAGGACCTGACCCTG |
| | | CTGAAAGCTCTCGTGCGGCAGCAGCTGCCTGAGAAGTACAAAGAGA |
| | | TTTTCTTCGACCAGAGCAAGAACGGCTACGCCGGCTACATTGACGG |
| | | CGGAGCCAGCCAGGAAGAGTTCTACAAGTTCATCAAGCCCATCCTG |
| | | GAAAAGATGGACGGCACCGAGGAACTGCTCGTGAAGCTGAACAGAG |
| | | AGGACCTGCTGCGGAAGCAGCGGACCTTCGACAACGGCAGCATCCC |
| | | CCACCAGATCCACCTGGGAGAGCTGCACGCCATTCTGCGGCGGCAG |
| | | GAAGATTTTTACCCATTCCTGAAGGACAACCGGGAAAAGATCGAGA |
| | | AGATCCTGACCTTCCGCATCCCCTACTACGTGGGCCCTCTGGCCAG |
| | | GGGAAACAGCAGATTCGCCTGGATGACCAGAAAGAGCGAGGAAACC |
| | | ATCACCCCCTGGAACTTCGAGGAAGTGGTGGACAAGGGCGCTTCCG |
| | | CCCAGAGCTTCATCGAGCGGATGACCAACTTCGATAAGAACCTGCC |
| | | CAACGAGAAGGTGCTGCCCAAGCACAGCCTGCTGTACGAGTACTTC |
| | | ACCGTGTATAACGAGCTGACCAAAGTGAAATACGTGACCGAGGGAA |
| | | TGAGAAAGCCCGCCTTCCTGAGCGGCGAGCAGAAAAAGGCCATCGT |
| | | GGACCTGCTGTTCAAGACCAACCGGAAAGTGACCGTGAAGCAGCTG |
| | | AAAGAGGACTACTTCAAGAAAATCGAGTGCTTCGACTCCGTGGAAA |
| | | TCTCCGGCGTGGAAGATCGGTTCAACGCCTCCCTGGGCACATACCA |
| | | CGATCTGCTGAAAATTATCAAGGACAAGGACTTCCTGGACAATGAG |
| | | GAAAACGAGGACATTCTGGAAGATATCGTGCTGACCCTGACACTGT |
| | | TTGAGGACAGAGAGATGATCGAGGAACGGCTGAAAACCTATGCCCA |
| | | CCTGTTCGACGACAAAGTGATGAAGCAGCTGAAGCGGCGGAGATAC |
| | | ACCGGCTGGGGCAGGCTGAGCCGGAAGCTGATCAACGGCATCCGGG |
| | | ACAAGCAGTCCGGCAAGACAATCCTGGATTTCCTGAAGTCCGACGG |
| | | CTTCGCCAACAGAAACTTCATGCAGCTGATCCACGACGACAGCCTG |
| | | ACCTTTAAAGAGGACATCCAGAAAGCCCAGGTGTCCGGCCAGGGCG |
| | | ATAGCCTGCACGAGCACATTGCCAATCTGGCCGGCAGCCCCGCCAT |
| | | TAAGAAGGGCATCCTGCAGACAGTGAAGGTGGTGGACGAGCTCGTG |
| | | AAAGTGATGGGCCGGCACAAGCCCGAGAACATCGTGATCGAAATGG |
| | | CCAGAGAGAACCAGACCACCCAGAAGGGACAGAAGAACAGCCGCGA |
| | | GAGAATGAAGCGGATCGAAGAGGGCATCAAAGAGCTGGGCAGCCAG |
| | | ATCCTGAAAGAACACCCCGTGGAAAACACCCAGCTGCAGAACGAGA |
| | | AGCTGTACCTGTACTACCTGCAGAATGGGCGGGATATGTACGTGGA |
| | | CCAGGAACTGGACATCAACCGGCTGTCCGACTACGATGTGGACGCC |
| | | ATCGTGCCTCAGAGCTTTCTGAAGGACGACTCCATCGACAACAAGG |
| | | TGCTGACCAGAAGCGACAAGAACCGGGGCAAGAGCGACAACGTGCC |
| | | CTCCGAAGAGGTCGTGAAGAAGATGAAGAACTACTGGCGGCAGCTG |
| | | CTGAACGCCAAGCTGATTACCCAGAGAAAGTTCGACAATCTGACCA |
| | | AGGCCGAGAGAGGCGGCCTGAGCGAACTGGATAAGGCCGGCTTCAT |
| | | CAAGAGACAGCTGGTGGAAACCCGGCAGATCACAAAGCACGTGGCA |
| | | CAGATCCTGGACTCCCGGATGAACACTAAGTACGACGAGAATGACA |
| | | AGCTGATCCGGGAAGTGAAAGTGATCACCCTGAAGTCCAAGCTGGT |
| | | GTCCGATTTCCGGAAGGATTTCCAGTTTTACAAAGTGCGCGAGATC |
| | | AACAACTACCACCACGCCCACGACGCCTACCTGAACGCCGTCGTGG |
| | | GAACCGCCCTGATCAAAAAGTACCCTAAGCTGGAAAGCGAGTTCGT |
| | | GTACGGCGACTACAAGGTGTACGACGTGCGGAAGATGATCGCCAAG |
| | | AGCGAGCAGGAAATCGGCAAGGCTACCGCCAAGTACTTCTTCTACA |
| | | GCAACATCATGAACTTTTTCAAGACCGAGATTACCCTGGCCAACGG |
| | | CGAGATCCGGAAGCGGCCTCTGATCGAGACAAACGGCGAAACCGGG |
| | | GAGATCGTGTGGGATAAGGGCCGGGATTTTGCCACCGTGCGGAAAG |
| | | TGCTGAGCATGCCCCAAGTGAATATCGTGAAAAAGACCGAGGTGCA |
| | | GACAGGCGGCTTCAGCAAAGAGTCTATCCTGCCCAAGAGGAACAGC |
| | | GATAAGCTGATCGCCAGAAAGAAGGACTGGGACCCTAAGAAGTACG |
| | | GCGGCTTCGACAGCCCCACCGTGGCCTATTCTGTGCTGGTGGTGGC |
| | | CAAAGTGGAAAAGGGCAAGTCCAAGAAACTGAAGAGTGTGAAAGAG |
| | | CTGCTGGGGATCACCATCATGGAAAGAAGCAGCTTCGAGAAGAATC |
| | | CCATCGACTTTCTGGAAGCCAAGGGCTACAAAGAAGTGAAAAAGGA |
| | | CCTGATCATCAAGCTGCCTAAGTACTCCCTGTTCGAGCTGGAAAAC |
| | | GGCCGGAAGAGAATGCTGGCCTCTGCCGGCGAACTGCAGAAGGGAA |
| | | ACGAACTGGCCCTGCCCTCCAAATATGTGAACTTCCTGTACCTGGC |
| | | CAGCCACTATGAGAAGCTGAAGGGCTCCCCCGAGGATAATGAGCAG |
| | | AAACAGCTGTTTGTGGAACAGCACAAGCACTACCTGGACGAGATCA |
| | | TCGAGCAGATCAGCGAGTTCTCCAAGAGAGTGATCCTGGCCGACGC |
| | | TAATCTGGACAAAGTGCTGTCCGCCTACAACAAGCACCGGGATAAG |
| | | CCCATCAGAGAGCAGGCCGAGAATATCATCCACCTGTTTACCCTGA |
| | | CCAATCTGGGAGCCCCTGCCGCCTTCAAGTACTTTGACACCACCAT |
| | | CGACCGGAAGAGGTACACCAGCACCAAAGAGGTGCTGGACGCCACC |
| | | CTGATCCACCAGAGCATCACCGGCCTGTACGAGACACGGATCGACC |
| | | TGTCTCAGCTGGGAGGCGACAGCCCCAAGAAGAAGAGAAAGGTGGG |
| | | AGTCGACGGATCCAGCGGCTCCGAGACCCCAGGCACATCTGAGAGC |
| | | GCCACCCCTGAGTCCACCGGTATGAACAATTCACAGGGGAGAGTGA |
| | | CATTCGAAGACGTGACCGTGAACTTCACCCAGGGAGAATGGCAGCG |
| | | CTTGAACCCAGAACAAAGGAACCTCTATCGGGACGTGATGCTGGAA |
| | | AACTACTCAAATTTGGTGAGCGTTGGGCAGGGTGAGACCACTAAGC |
| | | CTGACGTGATCCTGAGATTGGAACAGGGCAAGGAGCCTTGGCTCGA |
| | | GGAAGAGGAAGTCCTGGGCTCAGGGAGGGCCGAGAAAAACGGTGAT |
| | | ATAGGAGGCCAGATATGGAAGCCTAAGGACGTCAAGGAGAGCCTGA |
| | | GCGCTCCCAAGAAGAAAAGGAAGGTCCCAAAGAAAAAAAGAAAGGT |
| | | GTGAGGATCCTGAGTCTAGAAATCAACCTCTGGATTACAAAATTTG |
| | | TGAAAGATTGACTGGTATTCTTAACTATGTTGCTCCTTTTACGCTA |
| | | TGTGGATACGCTGCTTTAATGCCTTTGTATCATGCTATTGCTTCCC |
| | | GTATGGCTTTCATTTTCTCCTCCTTGTATAAATCCTGGTTGCTGTC |
| | | TCTTTATGAGGAGTTGTGGCCCGTTGTCAGGCAACGTGGCGTGGTG |
| | | TGCACTGTGTTTGCTGACGCAACCCCCACTGGTTGGGGCATTGCCA |
| | | CCACCTGTCAGCTCCTTTCCGGGACTTTCGCTTTCCCCCTCCCTAT |
| | | TGCCACGGCGGAACTCATCGCCGCCTGCCTTGCCCGCTGCTGGACA |
| | | GGGGCTCGGCTGTTGGGCACTGACAATTCCGTGGTGTTGTCGGGGA |
| | | AATCATCGTCCTTTCCTTGGCTGCTCGCCTGTGTTGCCACCTGGAT |
| | | TCTGCGCGGGACGTCCTTCTGCTACGTCCCTTCGGCCCTCAATCCA |
| | | GCGGACCTTCCTTCCCGCGGCCTGCTGCCGGCTCTGCGGCCTCTTC |
| | | CGCGTCTTCGCCTTCGCCCTCAGACGAGTCGGATCTCCCTTTGGGC |
| | | CGCCTCCCCGCCTGTTAATTAAAAAAAAAAAAAAAAAAAAAAAAAA |
| | | AAAAAAAAAAAAAAAAAAAAAAAAAAAAAAAAAAAAAAAAAAAAAA |
| | | AAAAAAAAAAAAAAAAAAAAAAAAAAAAAGCTTGAAGAGCCTAGTG |
| | | GCGCCTGATGCGGTATTTTCTCCTTACGCATCTGTGCGGTATTTCA |
| | | CACCGCATAATCCAGCACAGTGGCGGCCCGTTTAAACCCGCTGATC |
| | | AGCCTCGACTGTGCCTTCTAGTTGCCAGCCATCTGTTGTTTGCCCC |
| | | TCCCCCGTGCCTTCCTTGACCCTGGAAGGTGCCACTCCCACTGTCC |
| | | TTTCCTAATAAAATGAGGAAATTGCATCGCATTGTCTGAGTAGGTG |
| | | TCATTCTATTCTGGGGGGTGGGGTGGGGCAGGACAGCAAGGGGGAG |
| | | GATTGGGAAGACAATAGCAGGCATGCTGGGGATGCGGTGGGCTCTA |
| | | TGGCTTCTGAGGCGGAAAGAACCAGCTGCATTAATGAATCGGCCAA |
| | | CGCGCGGGGAGAGGCGGTTTGCGTATTGGGCGCTCTTCCGCTTCCT |
| | | CGCTCACTGACTCGCTGCGCTCGGTCGTTCGGCTGCGGCGAGCGGT |
| | | ATCAGCTCACTCAAAGGCGGTAATACGGTTATCCACAGAATCAGGG |
| | | GATAACGCAGGAAAGAACATGTGAGCAAAAGGCCAGCAAAAGGCCA |
| | | GGAACCGTAAAAAGGCCGCGTTGCTGGCGTTTTTCCATAGGCTCCG |
| | | CCCCCCTGACGAGCATCACAAAAATCGACGCTCAAGTCAGAGGTGG |
| | | CGAAACCCGACAGGACTATAAAGATACCAGGCGTTTCCCCCTGGAA |
| | | GCTCCCTCGTGCGCTCTCCTGTTCCGACCCTGCCGCTTACCGGATA |
| | | CCTGTCCGCCTTTCTCCCTTCGGGAAGCGTGGCGCTTTCTCATAGC |
| | | TCACGCTGTAGGTATCTCAGTTCGGTGTAGGTCGTTCGCTCCAAGC |
| | | TGGGCTGTGTGCACGAACCCCCCGTTCAGCCCGACCGCTGCGCCTT |
| | | ATCCGGTAACTATCGTCTTGAGTCCAACCCGGTAAGACACGACTTA |
| | | TCGCCACTGGCAGCAGCCACTGGTAACAGGATTAGCAGAGCGAGGT |
| | | ATGTAGGCGGTGCTACAGAGTTCTTGAAGTGGTGGCCTAACTACGG |
| | | CTACACTAGAAGAACAGTATTTGGTATCTGCGCTCTGCTGAAGCCA |
| | | GTTACCTTCGGAAAAAGAGTTGGTAGCTCTTGATCCGGCAAACAAA |
| | | CCACCGCTGGTAGCGGTGGTTTTTTTGTTTGCAAGCAGCAGATTAC |
| | | GCGCAGAAAAAAAGGATCTCAAGAAGATCCTTTGATCTTTTCTACG |
| | | GGGTCTGACGCTCAGTGGAACGAAAACTCACGTTAAGGGATTTTGG |
| | | TCATGAGATTATCAAAAAGGATCTTCACCTAGATCCTTTTAAATTA |
| | | AAAATGAAGTITTAAATCAATCTAAAGTATATATGAGTAAACTTGG |
| | | TCTGACAGTTAGAAAAACTCATCGAGCATCAAATGAAACTGCAATT |
| | | TATTCATATCAGGATTATCAATACCATATTTTTGAAAAAGCCGTTT |
| | | CTGTAATGAAGGAGAAAACTCACCGAGGCAGTTCCATAGGATGGCA |
| | | AGATCCTGGTATCGGTCTGCGATTCCGACTCGTCCAACATCAATAC |
| | | AACCTATTAATTTCCCCTCGTCAAAAATAAGGTTATCAAGTGAGAA |
| | | ATCACCATGAGTGACGACTGAATCCGGTGAGAATGGCAAAAGTTTA |
| | | TGCATTTCTTTCCAGACTTGTTCAACAGGCCAGCCATTACGCTCGT |
| | | CATCAAAATCACTCGCATCAACCAAACCGTTATTCATTCGTGATTG |
| | | CGCCTGAGCGAAACGAAATACGCGATCGCTGTTAAAAGGACAATTA |
| | | CAAACAGGAATCGAATGCAACCGGCGCAGGAACACTGCCAGCGCAT |
| | | CAACAATATTTTCACCTGAATCAGGATATTCTTCTAATACCTGGAA |
| | | TGCTGTTTTCCCAGGGATCGCAGTGGTGAGTAACCATGCATCATCA |
| | | GGAGTACGGATAAAATGCTTGATGGTCGGAAGAGGCATAAATTCCG |
| | | TCAGCCAGTTTAGTCTGACCATCTCATCTGTAACATCATTGGCAAC |
| | | GCTACCTTTGCCATGTTTCAGAAACAACTCTGGCGCATCGGGCTTC |
| | | CCATACAATCGATAGATTGTCGCACCTGATTGCCCGACATTATCGC |
| | | GAGCCCATTTATACCCATATAAATCAGCATCCATGTTGGAATTTAA |
| | | TCGCGGCCTAGAGCAAGACGTTTCCCGTTGAATATGGCTCATACTC |
| | | TTCCTTTTTCAATATTATTGAAGCATTTATCAGGGTTATTGTCTCA |
| | | TGAGCGGATACATATTTGAATGTATTTAGAAAAATAAACAAATAGG |
| | | GGTTCCGCGCACATTTCCCCGAAAAGTGCCACCTGACGTCGATCGA |
| | | CGGATCGGGAGATCTCCCGATCCCCTATGGTGCACTCTCAGTACAA |
| | | TCTGCTCTGATGCCGCATAGTTAAGCCAGTATCTGCTCCCTGCTTG |
| | | TGTGTTGGAGGTCGCTGAGTAGTGCGCGAGCAAAATTTAAGCTACA |
| | | ACAAGGCAAGGCTTGACCGACAATTGCATGAAGAATCTGCTTAGGG |
| | | TTAGGCGTTTTGCGCTGCTTCGCGATGTACGGGCCAGATATACGCG |
| | | TTGACATTGATTATTGACTAGTTATTAATAGTAATCAATTACGGGG |
| | | TCATTAGTTCATAGCCCATATATGGAGTTCCGCGTTACATAACTTA |
| | | CGGTAAATGGCCCGCCTGGCTGACCGCCCAACGACCCCCGCCCATT |
| | | GACGTCAATAATGACGTATGTTCCCATAGTAACGCCAATAGGGACT |
| | | TTCCATTGACGTCAATGGGTGGAGTATTTACGGTAAACTGCCCACT |
| | | TGGCAGTACATCAAGTGTATCATATGCCAAGTACGCCCCCTATTGA |
| | | CGTCAATGACGGTAAATGGCCCGCCTGGCATTATGCCCAGTACATG |
| | | ACCTTATGGGACTTTCCTACTTGGCAGTACATCTACGTATTAGTCA |
| | | TCGCTATTACCATGGTGATGCGGTTTTGGCAGTACATCAATGGGCG |
| | | TGGATAGCGGTTTGACTCACGGGGATTTCCAAGTCTCCACCCCATT |
| | | GACGTCAATGGGAGTTTGTTTTGGCACCAAAATCAACGGGACTTTC |
| | | CAAAATGTCGTAACAACTCCGCCCCATTGACGCAAATGGGCGGTAG |
| | | GCGTGTACGGTGGGAGGTCTATATAAGCAGAGCTCTCTGGCTAACT |
| | | AGAGAACCCACTGCTTACTGGCTTATCGAAATTAATACGACTCACT |
| | | ATAAG |
| Fusion Protein | 39 | MPKKKRKVPKKKRKVYNHDQEFDPPKVYPPVPAEKRKPIRVLSLFD |
| 2 | | GIATGLLVLKDLGIQVDRYIASEVCEDSITVGMVRHQGKIMYVGDV |
| | | RSVTQKHIQEWGPFDLVIGGSPCNDLSIVNPARKGLYEGTGRLFFE |
| | | FYRLLHDARPKEGDDRPFFWLFENVVAMGVSDKRDISRFLESNPVM |
| | | IDAKEVSAAHRARYFWGNLPGMNRPLASTVNDKLELQECLEHGRIA |
| | | KFSKVRTITTRSNSIKQGKDQHFPVFMNEKEDILWCTEMERVFGFP |
| | | VHYTDVSNMSRLARQRLLGRSWSVPVIRHLFAPLKEYFACVSSGNS |
| | | NANSRGPSFSSGLVPLSLRGSHMAAIPALDPEAEPSMDVILVGSSE |
| | | LSSSVSPGTGRDLIAYEVKANQRNIEDICICCGSLQVHTQHPLFEG |
| | | GICAPCKDKFLDALFLYDDDGYQSYCSICCSGETLLICGNPDCTRC |
| | | YCFECVDSLVGPGTSGKVHAMSNWVCYLCLPSSRSGLLQRRRKWRS |
| | | QLKAFYDRESENPLEMFETVPVWRRQPVRVLSLFEDIKKELTSLGF |
| | | LESGSDPGQLKHVVDVTDTVRKDVEEWGPFDLVYGATPPLGHTCDR |
| | | PPSWYLFQFHRLLQYARPKPGSPRPFFWMFVDNLVLNKEDLDVASR |
| | | FLEMEPVTIPDVHGGSLQNAVRVWSNIPAIRSRHWALVSEEELSLL |
| | | AQNKQSSKLAAKWPTKLVKNCFLPLREYFKYFSTELTSSLGGPSSG |
| | | APPPSGGSPAGSPTSTEEGTSESATPESGPGTSTEPSEGSAPGSPA |
| | | GSPTSTEEGTSTEPSEGSAPGTSTEPSELEDKKYSIGLAIGTNSVG |
| | | WAVITDEYKVPSKKFKVLGNTDRHSIKKNLIGALLFDSGETAEATR |
| | | LKRTARRRYTRRKNRICYLQEIFSNEMAKVDDSFFHRLEESFLVEE |
| | | DKKHERHPIFGNIVDEVAYHEKYPTIYHLRKKLVDSTDKADLRLIY |
| | | LALAHMIKFRGHFLIEGDLNPDNSDVDKLFIQLVQTYNQLFEENPI |
| | | NASGVDAKAILSARLSKSRRLENLIAQLPGEKKNGLFGNLIALSLG |
| | | LTPNFKSNFDLAEDAKLQLSKDTYDDDLDNLLAQIGDQYADLFLAA |
| | | KNLSDAILLSDILRVNTEITKAPLSASMIKRYDEHHQDLTLLKALV |
| | | RQQLPEKYKEIFFDQSKNGYAGYIDGGASQEEFYKFIKPILEKMDG |
| | | TEELLVKLNREDLLRKQRTFDNGSIPHQIHLGELHAILRRQEDFYP |
| | | FLKDNREKIEKILTFRIPYYVGPLARGNSRFAWMTRKSEETITPWN |
| | | FEEVVDKGASAQSFIERMTNFDKNLPNEKVLPKHSLLYEYFTVYNE |
| | | LTKVKYVTEGMRKPAFLSGEQKKAIVDLLFKTNRKVTVKQLKEDYF |
| | | KKIECFDSVEISGVEDRFNASLGTYHDLLKIIKDKDFLDNEENEDI |
| | | LEDIVLTLTLFEDREMIEERLKTYAHLFDDKVMKQLKRRRYTGWGR |
| | | LSRKLINGIRDKQSGKTILDFLKSDGFANRNFMQLIHDDSLTFKED |
| | | IQKAQVSGQGDSLHEHIANLAGSPAIKKGILQTVKVVDELVKVMGR |
| | | HKPENIVIEMARENQTTQKGQKNSRERMKRIEEGIKELGSQILKEH |
| | | PVENTQLQNEKLYLYYLQNGRDMYVDQELDINRLSDYDVDAIVPQS |
| | | FLKDDSIDNKVLTRSDKNRGKSDNVPSEEVVKKMKNYWRQLLNAKL |
| | | ITQRKFDNLTKAERGGLSELDKAGFIKRQLVETRQITKHVAQILDS |
| | | RMNTKYDENDKLIREVKVITLKSKLVSDFRKDFQFYKVREINNYHH |
| | | AHDAYLNAVVGTALIKKYPKLESEFVYGDYKVYDVRKMIAKSEQEI |
| | | GKATAKYFFYSNIMNFFKTEITLANGEIRKRPLIETNGETGEIVWD |
| | | KGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGIT |
| | | IMERSSFEKNPIDFLEAKGYKEVKKDLIIKLPKYSLFELENGRKRM |
| | | LASAGELQKGNELALPSKYVNFLYLASHYEKLKGSPEDNEQKQLFV |
| | | EQHKHYLDEIIEQISEFSKRVILADANLDKVLSAYNKHRDKPIREQ |
| | | AENIIHLFTLTNLGAPAAFKYFDTTIDRKRYTSTKEVLDATLIHQS |
| | | ITGLYETRIDLSQLGGDSPKKKRKVGVDGSSGSETPGTSESATPES |
| | | TGMNNSQGRVTFEDVTVNFTQGEWQRLNPEQRNLYRDVMLENYSNL |
| | | VSVGQGETTKPDVILRLEQGKEPWLEEEEVLGSGRAEKNGDIGGQI |
| | | WKPKDVKESLSAPKKKRKVPKKKRKV |
| Fusion Protein | 40 | GGGCGCTCGAGCAGGTTCAGAAGGAGATCAAAAACCCCCAAGGATC |
| 2 plasmid | | AAACATGCCAAAAAAGAAGAGAAAGGTACCGAAGAAAAAAAGAAAG |
| sequence | | GTATACAATCACGATCAGGAGTTCGACCCCCCTAAGGTGTACCCAC |
| | | CAGTGCCTGCAGAGAAGAGGAAGCCAATCCGGGTGCTGAGCCTGTT |
| | | TGATGGCATCGCCACCGGCCTGCTGGTGCTGAAGGATCTGGGCATC |
| | | CAGGTGGACCGGTACATCGCCTCCGAGGTGTGCGAGGATTCTATCA |
| | | CCGTGGGCATGGTGCGCCACCAGGGCAAGATCATGTATGTGGGCGA |
| | | CGTGCGGTCCGTGACACAGAAGCACATCCAGGAGTGGGGCCCATTC |
| | | GATCTGGTGATCGGCGGCAGCCCCTGTAATGACCTGTCCATCGTGA |
| | | ACCCTGCAAGGAAGGGACTGTACGAGGGAACCGGCCGGCTGTTCTT |
| | | TGAGTTTTATAGACTGCTGCACGACGCCAGGCCTAAGGAGGGCGAC |
| | | GATAGACCATTCTTTTGGCTGTTCGAGAATGTGGTGGCTATGGGCG |
| | | TGAGCGATAAGAGGGACATCTCCAGGTTTCTGGAGTCTAACCCCGT |
| | | GATGATCGATGCAAAGGAGGTGTCCGCCGCACACAGAGCCAGGTAT |
| | | TTCTGGGGCAATCTGCCAGGAATGAACAGGCCACTGGCAAGCACCG |
| | | TGAATGACAAGCTGGAGCTGCAGGAGTGCCTGGAGCACGGAAGGAT |
| | | CGCCAAGTTTTCCAAGGTGCGCACAATCACCACACGGAGCAATTCC |
| | | ATCAAGCAGGGCAAGGATCAGCACTTCCCCGTGTTCATGAACGAGA |
| | | AGGAGGACATCCTGTGGTGTACCGAGATGGAGAGAGTGTTCGGCTT |
| | | TCCAGTGCACTACACAGACGTGTCTAACATGAGCAGGCTGGCAAGG |
| | | CAGCGGCTGCTGGGCAGATCTTGGAGCGTGCCCGTGATCAGGCACC |
| | | TGTTCGCCCCTCTGAAGGAGTATTTTGCCTGCGTGAGCAGCGGCAA |
| | | CTCCAATGCCAACAGCCGGGGCCCCTCTTTCAGCTCCGGATTGGTG |
| | | CCTCTGAGCCTGAGGGGCTCCCACATGGCAGCAATCCCCGCCCTGG |
| | | ACCCCGAGGCCGAGCCTAGCATGGACGTGATCCTGGTGGGCTCTAG |
| | | CGAGCTGTCCTCTAGCGTGTCTCCAGGAACCGGAAGGGATCTGATC |
| | | GCATACGAGGTGAAGGCCAATCAGCGGAACATCGAGGACATCTGTA |
| | | TCTGCTGTGGCAGCCTGCAGGTGCACACACAGCACCCACTGTTCGA |
| | | GGGAGGAATCTGCGCACCCTGTAAGGATAAGTTCCTGGACGCCCTG |
| | | TTTCTGTACGACGATGACGGCTACCAGTCCTATTGCTCTATCTGCT |
| | | GTTCCGGCGAGACCCTGCTGATCTGCGGCAATCCAGATTGTACAAG |
| | | GTGCTATTGTTTTGAGTGCGTGGACTCTCTGGTGGGACCAGGCACC |
| | | AGCGGAAAGGTGCACGCCATGTCCAACTGGGTGTGCTACCTGTGCC |
| | | TGCCATCCTCTCGCAGCGGACTGCTGCAGCGGAGAAGGAAGTGGAG |
| | | ATCCCAGCTGAAGGCCTTCTATGATAGGGAGTCTGAGAACCCCCTG |
| | | GAGATGTTTGAGACCGTGCCAGTGTGGCGCCGGCAGCCCGTGAGGG |
| | | TGCTGAGCCTGTTCGAGGATATCAAGAAGGAGCTGACATCCCTGGG |
| | | CTTTCTGGAGTCCGGCTCTGACCCCGGACAGCTGAAGCACGTGGTG |
| | | GATGTGACCGACACAGTGCGGAAGGATGTGGAGGAGTGGGGCCCTT |
| | | TCGACCTGGTGTACGGAGCAACCCCTCCACTGGGACACACATGCGA |
| | | CAGACCCCCTTCTTGGTACCTGTTCCAGTTTCACCGCCTGCTGCAG |
| | | TATGCAAGGCCAAAGCCAGGCAGCCCTAGACCATTCTTTTGGATGT |
| | | TCGTGGATAATCTGGTGCTGAACAAGGAGGATCTGGACGTGGCCAG |
| | | CAGGTTTCTGGAGATGGAGCCAGTGACCATCCCAGACGTGCACGGC |
| | | GGCTCCCTGCAGAATGCCGTGCGCGTGTGGTCTAACATCCCTGCCA |
| | | TCAGAAGCAGGCACTGGGCACTGGTGAGCGAGGAGGAGCTGTCCCT |
| | | GCTGGCCCAGAATAAGCAGAGCAGCAAGCTGGCCGCCAAGTGGCCT |
| | | ACAAAGCTGGTGAAGAACTGCTTCCTGCCACTGCGGGAGTACTTCA |
| | | AGTATTTTTCCACCGAGCTGACATCTAGCCTGGGAGGACCCTCCTC |
| | | TGGCGCCCCACCACCTAGCGGCGGCTCCCCTGCCGGCTCTCCAACC |
| | | AGCACAGAGGAGGGCACCAGCGAGTCCGCCACACCAGAGTCTGGAC |
| | | CTGGCACCAGCACAGAGCCATCCGAGGGCTCTGCCCCAGGCTCTCC |
| | | TGCAGGCAGCCCTACCTCCACCGAAGAGGGCACCAGCACAGAGCCT |
| | | TCTGAGGGCAGCGCCCCAGGCACCTCTACAGAGCCAAGCGAGCTCG |
| | | AGGACAAGAAGTACAGCATCGGCCTGGCCATCGGCACCAACTCTGT |
| | | GGGCTGGGCCGTGATCACCGACGAGTACAAGGTGCCCAGCAAGAAA |
| | | TTCAAGGTGCTGGGCAACACCGACCGGCACAGCATCAAGAAGAACC |
| | | TGATCGGAGCCCTGCTGTTCGACAGCGGCGAAACAGCCGAGGCCAC |
| | | CCGGCTGAAGAGAACCGCCAGAAGAAGATACACCAGACGGAAGAAC |
| | | CGGATCTGCTATCTGCAAGAGATCTTCAGCAACGAGATGGCCAAGG |
| | | TGGACGACAGCTTCTTCCACAGACTGGAAGAGTCCTTCCTGGTGGA |
| | | AGAGGATAAGAAGCACGAGCGGCACCCCATCTTCGGCAACATCGTG |
| | | GACGAGGTGGCCTACCACGAGAAGTACCCCACCATCTACCACCTGA |
| | | GAAAGAAACTGGTGGACAGCACCGACAAGGCCGACCTGCGGCTGAT |
| | | CTATCTGGCCCTGGCCCACATGATCAAGTTCCGGGGCCACTTCCTG |
| | | ATCGAGGGCGACCTGAACCCCGACAACAGCGACGTGGACAAGCTGT |
| | | TCATCCAGCTGGTGCAGACCTACAACCAGCTGTTCGAGGAAAACCC |
| | | CATCAACGCCAGCGGCGTGGACGCCAAGGCCATCCTGTCTGCCAGA |
| | | CTGAGCAAGAGCAGACGGCTGGAAAATCTGATCGCCCAGCTGCCCG |
| | | GCGAGAAGAAGAATGGCCTGTTCGGCAACCTGATTGCCCTGAGCCT |
| | | GGGCCTGACCCCCAACTTCAAGAGCAACTTCGACCTGGCCGAGGAT |
| | | GCCAAACTGCAGCTGAGCAAGGACACCTACGACGACGACCTGGACA |
| | | ACCTGCTGGCCCAGATCGGCGACCAGTACGCCGACCTGTTTCTGGC |
| | | CGCCAAGAACCTGTCCGACGCCATCCTGCTGAGCGACATCCTGAGA |
| | | GTGAACACCGAGATCACCAAGGCCCCCCTGAGCGCCTCTATGATCA |
| | | AGAGATACGACGAGCACCACCAGGACCTGACCCTGCTGAAAGCTCT |
| | | CGTGCGGCAGCAGCTGCCTGAGAAGTACAAAGAGATTTTCTTCGAC |
| | | CAGAGCAAGAACGGCTACGCCGGCTACATTGACGGCGGAGCCAGCC |
| | | AGGAAGAGTTCTACAAGTTCATCAAGCCCATCCTGGAAAAGATGGA |
| | | CGGCACCGAGGAACTGCTCGTGAAGCTGAACAGAGAGGACCTGCTG |
| | | CGGAAGCAGCGGACCTTCGACAACGGCAGCATCCCCCACCAGATCC |
| | | ACCTGGGAGAGCTGCACGCCATTCTGCGGCGGCAGGAAGATTTTTA |
| | | CCCATTCCTGAAGGACAACCGGGAAAAGATCGAGAAGATCCTGACC |
| | | TTCCGCATCCCCTACTACGTGGGCCCTCTGGCCAGGGGAAACAGCA |
| | | GATTCGCCTGGATGACCAGAAAGAGCGAGGAAACCATCACCCCCTG |
| | | GAACTTCGAGGAAGTGGTGGACAAGGGCGCTTCCGCCCAGAGCTTC |
| | | ATCGAGCGGATGACCAACTTCGATAAGAACCTGCCCAACGAGAAGG |
| | | TGCTGCCCAAGCACAGCCTGCTGTACGAGTACTTCACCGTGTATAA |
| | | CGAGCTGACCAAAGTGAAATACGTGACCGAGGGAATGAGAAAGCCC |
| | | GCCTTCCTGAGCGGCGAGCAGAAAAAGGCCATCGTGGACCTGCTGT |
| | | TCAAGACCAACCGGAAAGTGACCGTGAAGCAGCTGAAAGAGGACTA |
| | | CTTCAAGAAAATCGAGTGCTTCGACTCCGTGGAAATCTCCGGCGTG |
| | | GAAGATCGGTTCAACGCCTCCCTGGGCACATACCACGATCTGCTGA |
| | | AAATTATCAAGGACAAGGACTTCCTGGACAATGAGGAAAACGAGGA |
| | | CATTCTGGAAGATATCGTGCTGACCCTGACACTGTTTGAGGACAGA |
| | | GAGATGATCGAGGAACGGCTGAAAACCTATGCCCACCTGTTCGACG |
| | | ACAAAGTGATGAAGCAGCTGAAGCGGCGGAGATACACCGGCTGGGG |
| | | CAGGCTGAGCCGGAAGCTGATCAACGGCATCCGGGACAAGCAGTCC |
| | | GGCAAGACAATCCTGGATTTCCTGAAGTCCGACGGCTTCGCCAACA |
| | | GAAACTTCATGCAGCTGATCCACGACGACAGCCTGACCTTTAAAGA |
| | | GGACATCCAGAAAGCCCAGGTGTCCGGCCAGGGCGATAGCCTGCAC |
| | | GAGCACATTGCCAATCTGGCCGGCAGCCCCGCCATTAAGAAGGGCA |
| | | TCCTGCAGACAGTGAAGGTGGTGGACGAGCTCGTGAAAGTGATGGG |
| | | CCGGCACAAGCCCGAGAACATCGTGATCGAAATGGCCAGAGAGAAC |
| | | CAGACCACCCAGAAGGGACAGAAGAACAGCCGCGAGAGAATGAAGC |
| | | GGATCGAAGAGGGCATCAAAGAGCTGGGCAGCCAGATCCTGAAAGA |
| | | ACACCCCGTGGAAAACACCCAGCTGCAGAACGAGAAGCTGTACCTG |
| | | TACTACCTGCAGAATGGGCGGGATATGTACGTGGACCAGGAACTGG |
| | | ACATCAACCGGCTGTCCGACTACGATGTGGACGCCATCGTGCCTCA |
| | | GAGCTTTCTGAAGGACGACTCCATCGACAACAAGGTGCTGACCAGA |
| | | AGCGACAAGAACCGGGGCAAGAGCGACAACGTGCCCTCCGAAGAGG |
| | | TCGTGAAGAAGATGAAGAACTACTGGCGGCAGCTGCTGAACGCCAA |
| | | GCTGATTACCCAGAGAAAGTTCGACAATCTGACCAAGGCCGAGAGA |
| | | GGCGGCCTGAGCGAACTGGATAAGGCCGGCTTCATCAAGAGACAGC |
| | | TGGTGGAAACCCGGCAGATCACAAAGCACGTGGCACAGATCCTGGA |
| | | CTCCCGGATGAACACTAAGTACGACGAGAATGACAAGCTGATCCGG |
| | | GAAGTGAAAGTGATCACCCTGAAGTCCAAGCTGGTGTCCGATTTCC |
| | | GGAAGGATTTCCAGTTTTACAAAGTGCGCGAGATCAACAACTACCA |
| | | CCACGCCCACGACGCCTACCTGAACGCCGTCGTGGGAACCGCCCTG |
| | | ATCAAAAAGTACCCTAAGCTGGAAAGCGAGTTCGTGTACGGCGACT |
| | | ACAAGGTGTACGACGTGCGGAAGATGATCGCCAAGAGCGAGCAGGA |
| | | AATCGGCAAGGCTACCGCCAAGTACTTCTTCTACAGCAACATCATG |
| | | AACTTTTTCAAGACCGAGATTACCCTGGCCAACGGCGAGATCCGGA |
| | | AGCGGCCTCTGATCGAGACAAACGGCGAAACCGGGGAGATCGTGTG |
| | | GGATAAGGGCCGGGATTTTGCCACCGTGCGGAAAGTGCTGAGCATG |
| | | CCCCAAGTGAATATCGTGAAAAAGACCGAGGTGCAGACAGGCGGCT |
| | | TCAGCAAAGAGTCTATCCTGCCCAAGAGGAACAGCGATAAGCTGAT |
| | | CGCCAGAAAGAAGGACTGGGACCCTAAGAAGTACGGCGGCTTCGAC |
| | | AGCCCCACCGTGGCCTATTCTGTGCTGGTGGTGGCCAAAGTGGAAA |
| | | AGGGCAAGTCCAAGAAACTGAAGAGTGTGAAAGAGCTGCTGGGGAT |
| | | CACCATCATGGAAAGAAGCAGCTTCGAGAAGAATCCCATCGACTTT |
| | | CTGGAAGCCAAGGGCTACAAAGAAGTGAAAAAGGACCTGATCATCA |
| | | AGCTGCCTAAGTACTCCCTGTTCGAGCTGGAAAACGGCCGGAAGAG |
| | | AATGCTGGCCTCTGCCGGCGAACTGCAGAAGGGAAACGAACTGGCC |
| | | CTGCCCTCCAAATATGTGAACTTCCTGTACCTGGCCAGCCACTATG |
| | | AGAAGCTGAAGGGCTCCCCCGAGGATAATGAGCAGAAACAGCTGTT |
| | | TGTGGAACAGCACAAGCACTACCTGGACGAGATCATCGAGCAGATC |
| | | AGCGAGTTCTCCAAGAGAGTGATCCTGGCCGACGCTAATCTGGACA |
| | | AAGTGCTGTCCGCCTACAACAAGCACCGGGATAAGCCCATCAGAGA |
| | | GCAGGCCGAGAATATCATCCACCTGTTTACCCTGACCAATCTGGGA |
| | | GCCCCTGCCGCCTTCAAGTACTTTGACACCACCATCGACCGGAAGA |
| | | GGTACACCAGCACCAAAGAGGTGCTGGACGCCACCCTGATCCACCA |
| | | GAGCATCACCGGCCTGTACGAGACACGGATCGACCTGTCTCAGCTG |
| | | GGAGGCGACAGCCCCAAGAAGAAGAGAAAGGTGGGAGTCGACGGAT |
| | | CCAGCGGCTCCGAGACCCCAGGCACATCTGAGAGCGCCACCCCTGA |
| | | GTCCACCGGTATGAACAATTCACAGGGGAGAGTGACATTCGAAGAC |
| | | GTGACCGTGAACTTCACCCAGGGAGAATGGCAGCGCTTGAACCCAG |
| | | AACAAAGGAACCTCTATCGGGACGTGATGCTGGAAAACTACTCAAA |
| | | TTTGGTGAGCGTTGGGCAGGGTGAGACCACTAAGCCTGACGTGATC |
| | | CTGAGATTGGAACAGGGCAAGGAGCCTTGGCTCGAGGAAGAGGAAG |
| | | TCCTGGGCTCAGGGAGGGCCGAGAAAAACGGTGATATAGGAGGCCA |
| | | GATATGGAAGCCTAAGGACGTCAAGGAGAGCCTGAGCGCTCCCAAG |
| | | AAGAAAAGGAAGGTCCCAAAGAAAAAAAGAAAGGTGTGAGGATCCT |
| | | GAGTCTAGAAATCAACCTCTGGATTACAAAATTTGTGAAAGATTGA |
| | | CTGGTATTCTTAACTATGTTGCTCCTTTTACGCTATGTGGATACGC |
| | | TGCTTTAATGCCTTTGTATCATGCTATTGCTTCCCGTATGGCTTTC |
| | | ATTTTCTCCTCCTTGTATAAATCCTGGTTGCTGTCTCTTTATGAGG |
| | | AGTTGTGGCCCGTTGTCAGGCAACGTGGCGTGGTGTGCACTGTGTT |
| | | TGCTGACGCAACCCCCACTGGTTGGGGCATTGCCACCACCTGTCAG |
| | | CTCCTTTCCGGGACTTTCGCTTTCCCCCTCCCTATTGCCACGGCGG |
| | | AACTCATCGCCGCCTGCCTTGCCCGCTGCTGGACAGGGGCTCGGCT |
| | | GTTGGGCACTGACAATTCCGTGGTGTTGTCGGGGAAATCATCGTCC |
| | | TTTCCTTGGCTGCTCGCCTGTGTTGCCACCTGGATTCTGCGCGGGA |
| | | CGTCCTTCTGCTACGTCCCTTCGGCCCTCAATCCAGCGGACCTTCC |
| | | TTCCCGCGGCCTGCTGCCGGCTCTGCGGCCTCTTCCGCGTCTTCGC |
| | | CTTCGCCCTCAGACGAGTCGGATCTCCCTTTGGGCCGCCTCCCCGC |
| | | CTGTTAATTAAAAAAAAAAAAAAAAAAAAAAAAAAAAAAAAAAAAA |
| | | AAAAAAAAAAAAAAAAAAAAAGCTTGAAGAGCCTAGTGGCGCCTGA |
| | | TGCGGTATTTTCTCCTTACGCATCTGTGCGGTATTTCACACCGCAT |
| | | AATCCAGCACAGTGGCGGCCCGTTTAAACCCGCTGATCAGCCTCGA |
| | | CTGTGCCTTCTAGTTGCCAGCCATCTGTTGTTTGCCCCTCCCCCGT |
| | | GCCTTCCTTGACCCTGGAAGGTGCCACTCCCACTGTCCTTTCCTAA |
| | | TAAAATGAGGAAATTGCATCGCATTGTCTGAGTAGGTGTCATTCTA |
| | | TTCTGGGGGGTGGGGTGGGGCAGGACAGCAAGGGGGAGGATTGGGA |
| | | AGACAATAGCAGGCATGCTGGGGATGCGGTGGGCTCTATGGCTTCT |
| | | GAGGCGGAAAGAACCAGCTGCATTAATGAATCGGCCAACGCGCGGG |
| | | GAGAGGCGGTTTGCGTATTGGGCGCTCTTCCGCTTCCTCGCTCACT |
| | | GACTCGCTGCGCTCGGTCGTTCGGCTGCGGCGAGCGGTATCAGCTC |
| | | ACTCAAAGGCGGTAATACGGTTATCCACAGAATCAGGGGATAACGC |
| | | AGGAAAGAACATGTGAGCAAAAGGCCAGCAAAAGGCCAGGAACCGT |
| | | AAAAAGGCCGCGTTGCTGGCGTTTTTCCATAGGCTCCGCCCCCCTG |
| | | ACGAGCATCACAAAAATCGACGCTCAAGTCAGAGGTGGCGAAACCC |
| | | GACAGGACTATAAAGATACCAGGCGTTTCCCCCTGGAAGCTCCCTC |
| | | GTGCGCTCTCCTGTTCCGACCCTGCCGCTTACCGGATACCTGTCCG |
| | | CCTTTCTCCCTTCGGGAAGCGTGGCGCTTTCTCATAGCTCACGCTG |
| | | TAGGTATCTCAGTTCGGTGTAGGTCGTTCGCTCCAAGCTGGGCTGT |
| | | GTGCACGAACCCCCCGTTCAGCCCGACCGCTGCGCCTTATCCGGTA |
| | | ACTATCGTCTTGAGTCCAACCCGGTAAGACACGACTTATCGCCACT |
| | | GGCAGCAGCCACTGGTAACAGGATTAGCAGAGCGAGGTATGTAGGC |
| | | GGTGCTACAGAGTTCTTGAAGTGGTGGCCTAACTACGGCTACACTA |
| | | GAAGAACAGTATTTGGTATCTGCGCTCTGCTGAAGCCAGTTACCTT |
| | | CGGAAAAAGAGTTGGTAGCTCTTGATCCGGCAAACAAACCACCGCT |
| | | GGTAGCGGTGGTTTTTTTGTTTGCAAGCAGCAGATTACGCGCAGAA |
| | | AAAAAGGATCTCAAGAAGATCCTTTGATCTTTTCTACGGGGTCTGA |
| | | CGCTCAGTGGAACGAAAACTCACGTTAAGGGATTTTGGTCATGAGA |
| | | TTATCAAAAAGGATCTTCACCTAGATCCTTTTAAATTAAAAATGAA |
| | | GTTTTAAATCAATCTAAAGTATATATGAGTAAACTTGGTCTGACAG |
| | | TTAGAAAAACTCATCGAGCATCAAATGAAACTGCAATTTATTCATA |
| | | TCAGGATTATCAATACCATATTTTTGAAAAAGCCGTTTCTGTAATG |
| | | AAGGAGAAAACTCACCGAGGCAGTTCCATAGGATGGCAAGATCCTG |
| | | GTATCGGTCTGCGATTCCGACTCGTCCAACATCAATACAACCTATT |
| | | AATTTCCCCTCGTCAAAAATAAGGTTATCAAGTGAGAAATCACCAT |
| | | GAGTGACGACTGAATCCGGTGAGAATGGCAAAAGTTTATGCATTTC |
| | | TTTCCAGACTTGTTCAACAGGCCAGCCATTACGCTCGTCATCAAAA |
| | | TCACTCGCATCAACCAAACCGTTATTCATTCGTGATTGCGCCTGAG |
| | | CGAAACGAAATACGCGATCGCTGTTAAAAGGACAATTACAAACAGG |
| | | AATCGAATGCAACCGGCGCAGGAACACTGCCAGCGCATCAACAATA |
| | | TTTTCACCTGAATCAGGATATTCTTCTAATACCTGGAATGCTGTTT |
| | | TCCCAGGGATCGCAGTGGTGAGTAACCATGCATCATCAGGAGTACG |
| | | GATAAAATGCTTGATGGTCGGAAGAGGCATAAATTCCGTCAGCCAG |
| | | TTTAGTCTGACCATCTCATCTGTAACATCATTGGCAACGCTACCTT |
| | | TGCCATGTTTCAGAAACAACTCTGGCGCATCGGGCTTCCCATACAA |
| | | TCGATAGATTGTCGCACCTGATTGCCCGACATTATCGCGAGCCCAT |
| | | TTATACCCATATAAATCAGCATCCATGTTGGAATTTAATCGCGGCC |
| | | TAGAGCAAGACGTTTCCCGTTGAATATGGCTCATACTCTTCCTTTT |
| | | TCAATATTATTGAAGCATTTATCAGGGTTATTGTCTCATGAGCGGA |
| | | TACATATTTGAATGTATTTAGAAAAATAAACAAATAGGGGTTCCGC |
| | | GCACATTTCCCCGAAAAGTGCCACCTGACGTCGATCGACGGATCGG |
| | | GAGATCTCCCGATCCCCTATGGTGCACTCTCAGTACAATCTGCTCT |
| | | GATGCCGCATAGTTAAGCCAGTATCTGCTCCCTGCTTGTGTGTTGG |
| | | AGGTCGCTGAGTAGTGCGCGAGCAAAATTTAAGCTACAACAAGGCA |
| | | AGGCTTGACCGACAATTGCATGAAGAATCTGCTTAGGGTTAGGCGT |
| | | TTTGCGCTGCTTCGCGATGTACGGGCCAGATATACGCGTTGACATT |
| | | GATTATTGACTAGTTATTAATAGTAATCAATTACGGGGTCATTAGT |
| | | TCATAGCCCATATATGGAGTTCCGCGTTACATAACTTACGGTAAAT |
| | | GGCCCGCCTGGCTGACCGCCCAACGACCCCCGCCCATTGACGTCAA |
| | | TAATGACGTATGTTCCCATAGTAACGCCAATAGGGACTTTCCATTG |
| | | ACGTCAATGGGTGGAGTATTTACGGTAAACTGCCCACTTGGCAGTA |
| | | CATCAAGTGTATCATATGCCAAGTACGCCCCCTATTGACGTCAATG |
| | | ACGGTAAATGGCCCGCCTGGCATTATGCCCAGTACATGACCTTATG |
| | | GGACTTTCCTACTTGGCAGTACATCTACGTATTAGTCATCGCTATT |
| | | ACCATGGTGATGCGGTTTTGGCAGTACATCAATGGGCGTGGATAGC |
| | | GGTTTGACTCACGGGGATTTCCAAGTCTCCACCCCATTGACGTCAA |
| | | TGGGAGTTTGTTTTGGCACCAAAATCAACGGGACTTTCCAAAATGT |
| | | CGTAACAACTCCGCCCCATTGACGCAAATGGGCGGTAGGCGTGTAC |
| | | GGTGGGAGGTCTATATAAGCAGAGCTCTCTGGCTAACTAGAGAACC |
| | | CACTGCTTACTGGCTTATCGAAATTAATACGACTCACTATAAG |
| Tracr encoding | 41 | GTTTTAGAGCTAGAAATAGCAAGTTAAAATAAGGCTAGTCCGTTAT |
| sequence 1 | | CAACTTGAAAAAGTGGCACCGAGTCGGTGCTTT |
| Tracr encoding | 42 | GTTTAAGAGCTAGAAATAGCAAGTTTAAATAAGGCTAGTCCGTTAT |
| sequence 2 | | CAACTTGAAAAAGTGGCACCGAGTCGGTGCTTTT |
| Tracr encoding | 43 | GTTTAAGAGCTAAGCTGGAAACAGCATAGCAAGTTTAAATAAGGCT |
| sequence 3 | | AGTCCGTTATCAACTTGAAAAAGTGGCACCGAGTCGGTGCTTTTTT |
| | | T |
| Tracr encoding | 44 | GTTTTAGTACTCTGGAAACAGAATCTACTAAAACAAGGCAAAATGC |
| sequence 4 | | CGTGTTTATCTCGTCAACTTGTTGGCGAGATTTT |
| Tracr 1 | 45 | GUUUAAGAGCUAAGCUGGAAACAGCAUAGCAAGUUUAAAUAAGGCU |
| | | AGUCCGUUAUCAACUUGAAAAAGUGGCACCGAGUCGGUGCUUUUUU |
| Tracr 2 | 46 | GUUUUAGAGCUAGAAAUAGCAAGUUAAAAUAAGGCUAGUCCGUUAU |
| | | CAACUUGAAAAAGUGGCACCGAGUCGGUGCUUUU |

### Editing System

Disclosed herein is an editing system, wherein a sgRNA disclosed herein (e.g., modified sgRNA) is able to form a complex with a CRISPR-associated protein. In some embodiments, the sgRNA disclosed herein (e.g., modified sgRNA) can form a ribonucleoprotein (RNP) with a nuclease (e.g., a Cas protein). In some embodiments, the RNP comprises Cas9 and gRNA. In some embodiments, the nuclease (e.g., a Cas protein) fuses to another protein or polypeptide heterologous to the Cas protein to create a fusion protein. In some embodiments, the nuclease (e.g., a Cas protein) fuses to one or more effector domains disclosed herein to form a Cas protein-effector domain fusion. In some embodiments, the sgRNA disclosed herein (e.g., modified sgRNA) complexes with a Cas protein-effector domain fusion.

In some embodiments, the editing system is a CRISPR system. The CRISPR system is derived advantageously from a type II CRISPR system. In some embodiments, one or more elements of a CRISPR system is derived from a particular organism comprising an endogenous CRISPR system, such as Streptococcus pyogenes. In some embodiments of the invention, the CRISPR system is a type II CRISPR system and the Cas enzyme is Cas9, which catalyzes DNA cleavage.

It will be appreciated that the terms Cas and CRISPR enzyme are generally used herein interchangeably, unless otherwise apparent. In some embodiments, the unmodified CRISPR enzyme, such as Cas9, has DNA cleavage activity. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, the CRISPR enzyme is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC 1 catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As a further example, two or more catalytic domains of Cas9 (RuvC I, RuvC II, and RuvC III or the HNH domain) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is about no more than 25%, 10%, 5%, 1%, 0.1%, 0.01%, or less of the DNA cleavage activity of the non-mutated form of the enzyme; an example can be when the DNA cleavage activity of the mutated form is nil or negligible as compared with the non-mutated form. Where the enzyme is not SpCas9, mutations may be made at any or all residues corresponding to positions 10, 762, 840, 854, 863 and/or 986 of SpCas9 (which may be ascertained for instance by standard sequence comparison tools). In particular, any or all of the following mutations can be made in SpCas9: D10A, E762A, H840A, N854A, N863A and/or D986A; as well as conservative substitution for any of the replacement amino acids is also envisaged. The same (or conservative substitutions of these mutations) at corresponding positions in other Cas9s can also be present. Orthologs of SpCas9 can be used in the practice of the invention. As mentioned above, many of the residue numberings used herein refer to the Cas9 enzyme from the type II CRISPR locus in Streptococcus pyogenes. However, it will be appreciated that the term “Cas9” includes many more Cas9s from other species of microbes, such as SpCas9, SaCa9, St1Cas9 and so forth. Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence (examples include NGG/NRG or a PAM that can be determined as described herein) following the 20 nucleotides of the target sequence. CRISPR activity through Cas9 for site-specific DNA recognition and cleavage is defined by the guide sequence, the tracr sequence that hybridizes in part to the guide sequence, and the PAM sequence. The type II CRISPR locus from Streptococcus pyogenes SF370 contains a cluster of four genes Cas9, Cas1, Cas2, and Csn1, as well as two non-coding RNA elements, tracrRNA, and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. A pre-crRNA array consisting of a single spacer flanked by two direct repeats (DRs) is also encompassed by the term “tracr-mate sequences”). In certain embodiments, Cas9 may be constitutively present or inducibly present or conditionally present or administered or delivered. Cas9 optimization may be used to enhance function or to develop new functions, one can generate chimeric Cas9 proteins. Cas9 may be used as a generic DNA binding protein.

### Cas Proteins

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof.

In some embodiments, an editing system herein comprises a Cas protein, e.g., a Cas9 protein domain. The Cas9 domain can be any of the Cas9 domains or Cas9 proteins (e.g., nuclease inactive Cas9 or Cas9 nickase, or a Cas9 variant from any species) provided herein. In some embodiments, any of the Cas domains or Cas proteins provided herein is fused with one or more any effector protein domain as described herein. In some embodiments, any of the Cas protein domains provided herein is fused with two or more effector protein domains as described herein. Cas9 can refer to a polypeptide with at least about 50%, 60%, 70%, 80%, 90%, 100% sequence identity and/or sequence similarity to a wild type exemplary Cas9 polypeptide (e.g., from S. pyogenes). Cas9 can refer to the wild type or a modified form of the Cas9 protein that comprises an amino acid change such as a deletion, insertion, substitution, variant, mutation, fusion, chimera, or any combination thereof.

Cas9 sequences and structures of variant Cas9 orthologs have been described in various species. Exemplary species that the Cas9 protein or other components can be from include, but are not limited to, Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polar omonas naphthalenivorans, Polar omonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionium, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillator ia sp., Petrotoga mobilis, Thermosipho africanus, Streptococcus pasteurianus, Neisseria cinerea, Campylobacter lari, Parvibaculum lavamentivorans, Coryne bacterium diphtheria, or Acaryochloris marina. In some embodiments, the Cas9 protein is from Streptococcus pyogenes. In some embodiments, the Cas9 protein may be from Streptococcus thermophilus. In some embodiments, the Cas9 protein is from Staphylococcus aureus.

Additional suitable Cas9 proteins, orthologs, variants, including nuclease inactive variants and sequences will be apparent to those of skill in the art based on this disclosure, and such Cas9 nucleases and sequences include Cas9 sequences from the organisms and loci disclosed in Chylinski et al., (2013) RNA Biology 10:5, 726-737; which are incorporated herein by reference.

### Epigenetic Editing Systems

In some embodiments, the editing system is an epigenetic editing system. An epigenetic editing system comprises a nuclease inactive Cas9 domain (dead Cas9 or dCas9). The dCas9 protein domain may comprise one, two, or more mutations as compared to a wild type Cas9 that abrogate its nuclease activity, but retains the DNA binding activity. For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9. In some embodiments, the dCas9 comprises at least one mutation in the HNH subdomain and the RuvC subdomain that reduces or abrogates nuclease activity. In some embodiments, the dCas9 only comprises a RuvC subdomain. In some embodiments, the dCas9 only comprises a HNR subdomain. It is to be understood that any mutation that inactivates the RuvC or the HNH domain may be included in a dCas9, e.g., insertion, deletion, or single or multiple amino acid substitution in the RuvC domain and/or the HNH domain.

Additional suitable mutations that inactivate Cas9 will be apparent to those of skill in the art based on this disclosure and knowledge in the field and are within the scope of this disclosure. Such additional exemplary suitable nuclease-inactive Cas9 domains include, but are not limited to, D839A, N863A, and/or K603R. Cas9, dCas9, or Cas9 variant also encompasses Cas9, dCas9, or Cas9 variants from any organism. Also appreciated is that dCas9, Cas9 nickase, or other appropriate Cas9 variants from any organisms can be used in accordance with the present disclosure.

In some embodiments, an epigenetic editing system comprises a high fidelity Cas9 domain. For example, high fidelity Cas9 domains comprising one or more mutations that decrease electrostatic interactions between the Cas9 domain and the sugar-phosphate backbone of DNA may be incorporated in an epigenetic editing system to confer increased target binding specificity as compared to a corresponding wild-type Cas9 domain. Without wishing to be bound by any particular theory, high fidelity Cas9 domains that have decreased electrostatic interactions with the sugar-phosphate backbone of DNA may have less off-target effects. In some embodiments, the Cas9 domain comprises one or more mutations that decreases the association between the Cas9 domain and the sugar-phosphate backbone of DNA. In some embodiments, a Cas9 domain comprises one or more mutations that decreases the association between the Cas9 domain and the sugar-phosphate backbone of DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, or more. In some embodiments, a high fidelity Cas9 domain comprises one or more of N497X, R661X, Q695X, and/or Q926X mutation as numbered in the wild type Cas9 amino acid sequence Uniprot Reference Sequence: Q99ZW2 or a corresponding amino acid in another Cas9, wherein X is any amino acid. In some embodiments, a high fidelity Cas9 domain comprises one or more of N497A, R661A, Q695A, and/or Q926A mutation of the amino acid sequence provided in the wild type Cas9 sequence, or a corresponding mutation as numbered in the wild type Cas9 amino acid sequence Uniprot Reference Sequence: Q99ZW2 or a corresponding amino acid in another Cas9. It should be appreciated that any of the epigenetic editing systems provided herein, for example, any of the epigenetic activators or repressors provided herein, can be converted into high fidelity epigenetic editing systems by modifying the Cas9 domain as described. In some embodiments, the high fidelity Cas9 domain is a nuclease inactive Cas9 domain.

In some embodiments, a DNA binding domain in a gene editing system is a CRISPR protein that recognizes a protospacer adjacent motif (PAM) sequence in a target gene. A CRISPR protein can recognize a naturally occurring or canonical PAM sequence or can have altered PAM specificities. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et ah. “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the Cas9 domain is a Cas9 domain from S. pyogenes (SpCas9). In some embodiments, a SpCas9 recognizes a canonical NGG PAM sequence where the “N” in “NGG” is adenine (A), thymine (T), guanine (G), or cytosine (C), and the G is guanine. In some embodiments, an epigenetic editing system or fusion protein provided herein contains a SpCas9 domain that is capable of binding a nucleotide sequence that does not contain a canonical (e.g., NGG) PAM sequence. In some embodiments, the SpCas9 domain, the nuclease inactive SpCas9 domain, or the SpCas9 nickase domain binds to a nucleic acid sequence having a NGG, a NGA, or a NGCG PAM sequence. In some embodiments, the Cas9 domain is a modified SpCas9 domain having specificity for a 5′-NGCG-3′ PAM sequence, where N is any one of nucleotides A, G, C, or T. In some embodiments, the Cas9 domain is a modified SpCas9 domain having specificity for a 5′-NGAN-3′ or a 5-NGNG-3′ PAM sequence, where N is any one of nucleotides A, G, C, or T. In some embodiments, the Cas9 domain is a modified SpCas9 domain having specificity for a 5′-NGN-3′ PAM sequence, where N is any one of nucleotides A, G, C. or T. In some embodiments, the Cas9 domain is a modified SpCas9 domain having specificity for a 5′-NRN-3′ or a 5′-NYN-3′ PAM sequence, where N is any one of nucleotides A, G, C, or T, where R is nucleotide A or G, and where Y is nucleotide C or T.

In some embodiments, the Cas9 domain is a Cas9 domain from Staphylococcus aureus (SaCas9). In some embodiments, the SaCas9 domain is a nuclease inactive SaCas9 (dSacas9).In some embodiments, the SaCas9 domain, the nuclease inactive SaCas9 domain, or the SaCas9 nickase domain binds to a nucleic acid sequence having a non-canonical PAM. In some embodiments, the SaCas9 domain, the SaCas9d domain, or the SaCas9n domain binds to a nucleic acid sequence having a NNGRRT PAM sequence, where N=A, T, C, or G, and R=A or G. In some embodiments, the Cas9 domain is a Cas9 domain from Neisseria meningitidis (NmeCas9). In some embodiments, the NmeCas9 domain is a nuclease inactive NmeCas9 (dNmeCas9). An NmeCas9 may have specificity for a 5′-NNNGATT-3′ PAM, where N is any one of nucleotides A, G. C, or T. In some embodiments, the Cas9 domain is a Cas9 domain from Campylobacter jejuni (CjCas9). In some embodiments, the CjCas9 domain is a nuclease inactive CjCas9 (dCjCas9). A Cj Cas9 can have specificity for a 5′-NNNVRYM-3′ PAM, where N is any one of nucleotides A, G, C, or T, V is nucleotide A, C, or G, R is nucleotide A or G, Y is nucleotide C or T, and M is nucleotide A or C. In some embodiments, the Cas9 domain is a Cas9 domain from Streptococcus thermophilus (StCas9). In some embodiments, the StCas9 is encoded by St CRISPR1 loci of the Streptococcus thermophilus (St1Cas9). In some embodiments, the St1Cas9 domain is a nuclease inactive St1 Cas9 (dSt1 Cas9). An St1 Cas9 has specificity for a 5′-NNAGAAW-3′ PAM, where N is any one of nucleotides A, G. C, or T, and W is nucleotide A or T. In some embodiments, the StCas9 is encoded by St CRISPR3 loci of the Streptococcus thermophilus (St3Cas9). In some embodiments, the St3Cas9 domain is a nuclease inactive St3Cas9 (dSt3Cas9). An St3Cas9 has specificity for a 5′-NGGNG-3′ PAM, where N is any one of nucleotides A, G. C, or T.

In some embodiments, the epigenetic editing system provided herein comprises a Cpf1 (or Cas12a) protein domain. For example, the epigenetic editing system comprises a nuclease inactive Cpf1 protein or a variant thereof. The Cpf1 protein has a RuvC-like endonuclease domain that is similar to the RuvC domain of Cas9 but does not have a HNH endonuclease domain, and the N-terminal of Cpf1 does not have the alpha-helical recognition lobe of Cas9. In some embodiments, the Cpf1 is a Cpf1 protein from Lachnospiraceae bacterium (LbCpf1). A LbCpf1 may have specificity for a 5′-TTTV-3′ PAM sequence, where V is any one of nucleotides A. G. or C. In some embodiments, the LbCpf1 protein has reduced nuclease activity. In some embodiments, the nuclease activity of the LbCpf1 protein is abolished (dLbCpf1). In some embodiments, the Cpf1 is a Cpf1 protein from Acidaminococcus sp. (AsCpf1). A AsCpf1 may have specificity for a 5′-TTTV-3′ PAM sequence, where V is any one of nucleotides A, G, or C. In some embodiments, the AsCpf1 protein has reduced nuclease activity. In some embodiments, the nuclease activity of the AsCpf1 protein is abolished (dAsCpf10. In some embodiments, the dAsCpf1 or AsCpf1 protein further comprises mutations that improve fidelity of target recognition of the protein. In some embodiments, the dAsCpf1 or AsCpf1 protein further comprises mutations that result in altered PAM specificity of the protein.

In some embodiments, an epigenetic editing system provided herein comprises a Cas protein domain other than Cas9. In some embodiments, the Cas9 protein comprises an inactivated nuclease domain. In some embodiments, an epigenetic editing system comprises a Cas12a, a Cas12b, a Cas12c, a Cas12d, a Cas12e, a Cas12h, or a Cas12i domain. In some embodiments, the Cas9 protein is an RNA nuclease or an inactivated RNA nuclease. In some embodiments, an epigenetic editing system comprises a Cas12g, a Cas13a, a Cas13b, a Cas13c, or a Cas13d domain. In some embodiments, an epigenetic editing system comprises an Argonaut protein domain.

A CRISPR/Cas system or a Cas protein in an epigenetic editing system provided herein comprises Class 1 or Class 2 Cas proteins. The Class 1 or Class 2 proteins used in an epigenetic editing system can be inactivated in its nuclease activity. In some embodiments, an epigenetic editing system comprises a Cas protein derived from a Type II, Type IIA, Type IIB, Type IIC, Type V, or Type VI Cas nuclease. In some embodiments, an epigenetic editing system comprises a Cas protein derived from a Class 2 Cas nucleases derived from Cas1, Cas1B, Cas2, Cas3, Cas4, Cas5, Cas6, Cas7, Cas8, Cas10, Cas14a, Cas14b, Cas14c, CasX, CasY, CasPhi, C2c4, C2c8, C2c9, C2c10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, or homologues or modified versions thereof. In some embodiments, a Cas protein in an epigenetic editing system is a nuclease inactivated Cas protein.

In some embodiments, the epigenetic editing system comprises a CasX (Cas12e) protein. A CasX protein has specificity for a 5′-TTCN-3′ PAM sequence, where N is any one of nucleotides A, G, T, or C. In some embodiments, the CasX protein has reduced or abolished nuclease activity (dCasX). In some embodiments, the epigenetic editing system comprises a CasY (Cas12d) protein. A CasY protein can have specificity for a 5′-TA-3′ PAM sequence. In some embodiments, the CasY protein has reduced or abolished nuclease activity (dCasY). In some embodiments, the epigenetic editing system comprises a Case (CasPhi) protein. A Case protein can have specificity for a 5′-TTN-3′ PAM sequence, wherein N is any one of nucleotides A, T, G, or C. In some embodiments, the Casp protein has reduced or abolished nuclease activity (dCasφ).

In some embodiments, the Cas protein is a circular permutant Cas protein. For example, an epigenetic editing system comprises a circular permutant Cas9 as described in Oakes et al., Cell 176, 254-267 (2019), incorporated herein in its entirety. As used herein, the term “circular permutant” refers to a variant polypeptide (e.g., of a subject Cas protein) in which one section of the primary amino acid sequence has been moved to a different position within the primary amino acid sequence of the polypeptide, but where the local order of amino acids has not been changed, and where the three dimensional architecture of the protein is conserved. For example, a circular permutant of a wild type 1000 amino acid polypeptide can have an N-terminal residue of residue number 500 (relative to the wild type protein), where residues 1-499 of the wild type protein are added the C-terminus. Such a circular permutant, relative to the wild type protein sequence would have, from N-terminus to C-terminus, amino acid numbers 500-1000 followed by 1-499, resulting in a circular permutant protein with amino acid 499 being the C-terminal residue. Thus, such an example circular permutant would have the same total number of amino acids as the wild type reference protein, and the amino acids would be in the same order locally in specific regions of the circular permutant, but the overall primary amino acid sequence is changed.

In some embodiments, an epigenetic editing system comprises a circular permuted Cas protein, e.g., a circular permuted Cas9 protein. In some embodiments, the epigenetic editing system comprises a fusion of a circular permuted Cas protein and an epigenetic effector domain, where the epigenetic effector domain is fused to the circular permuted Cas protein to a N-terminus or C-terminus that is different from that of wild type Cas protein.

In some embodiments, the circular permuted Cas protein comprises a N-terminal end of an N-terminal fragment of a wild type Cas protein fused to a C-terminus of a C-terminal fragment of the wild type Cas protein, hereby generating new N- and C-termini. Without wishing to be bound by any theory, the N-terminus and C-terminus of a wild type Cas protein may be locked in a small region, which may cause steric hinderance when the Cas protein is fused to an effect domain and reduced access to the target DNA sequence. In some embodiments, the epigenetic editing system comprising a circular permutant Cas protein has reduced steric incompatibility as compared to an epigenetic editing system comprising a wild type Cas protein counterpart. In some embodiments, the epigenetic editing system comprising a circular permutant Cas protein has improved effectiveness as compared to an epigenetic editing system comprising a wild type Cas protein counterpart. In some embodiments, the epigenetic editing system comprising a circular permutant Cas protein has improved epigenetic editing accuracy as compared to an epigenetic editing system comprising a wild type Cas protein counterpart. In some embodiments, the epigenetic editing system comprising a circular permutant Cas protein has reduced off-target editing effect as compared to an epigenetic editing system comprising a wild type Cas protein counterpart.

### Effector Domains

Epigenetic editing system disclosed herein can include one or more effector protein domains that modulate expression of a target gene. An effector domain can be used to contact a target polynucleotide sequence in a target gene to effect an epigenetic modification, for example, a change in methylation state of DNA nucleotides in the target gene. Accordingly, an epigenetic editing system with one or more effector domains can provide the effect of modulating expression of a target gene without altering the DNA sequence of the target gene. For example, in some embodiments, an effector domain results in repression or silencing of expression of a target gene. In some embodiments, an effector domain results in activation or increased expression of a target gene.

An epigenetic effector can deposit a chemical modification at the chromatin at the position of a target gene. Non limiting examples of chemical modifications include methylation, demethylation, acetylation, deacetylation, phosphorylation, SUMOylation and/or ubiquitination of the DNA or histone residues of the chromatin. In some embodiments, an epigenetic effector makes histone tail modifications. In some embodiments epigenetic effectors adds or removes active marks on histone tails. In some embodiments the active marks includes H3K4 methylation, H3K9 acetylation, H3K27 acetylation, H3K36 methylation, H3K79 methylation, H4K5 acetylation, H4K8 acetylation, H4K12 acetylation, H4K16 acetylation, and/or H4K20 methylation. In some embodiments epigenetic effectors adds or removes repressive marks on histone tails. In some embodiments these repressive marks includes H3K9 methylation and/or H3K27 methylation.

In some embodiments, an effector domain in an epigenetic editing system alters a chemical modification state of a target gene harboring a target sequence. For example, an effector domain can alter a chemical modification state of a nucleotide in the target gene. In some embodiments, an effector domain of an epigenetic editing system deposits a chemical modification at a nucleotide in the target gene. In some embodiments, an effector domain of an epigenetic editing system deposits a chemical modification of a histone associated with the target gene. In some embodiments, an effector domain of an epigenetic editing system removes a chemical modification at a nucleotide in the target gene. In some embodiments, an effector domain of an epigenetic editing system removes a chemical modification of a histone associated with the target gene. In some embodiments, the chemical modification increases expression of the target gene. For example, the epigenetic editing system comprises an effector domain having histone acetyltransferase activity. In some embodiments, the chemical modification decreases expression of the target gene. For example, the epigenetic editing system comprises an effector domain having DNA methyltransferase activity.

The epigenetic modification mediated by an epigenetic editing system can be in the vicinity of the target gene, or may be distant to the target gene, or spread from an initial epigenetic modification initiated by the epigenetic editing system at one or more nucleotide in a target sequence of the target gene.

In some embodiments, the alternation of the chemical modification state is a DNA methylation state. For example, methylation can be introduced by an effector domain having DNA methyltransferase activity or can be removed by an effector domain having DNA-demethylase activity. Alternatively, methylation can be introduced an effector domain having recruiting activity (e.g., DNMT3L) recruit an additional effector domain having DNA methyltransferase activity (e.g., DNMT3A/B). In some embodiments, the alteration of chemical modification, e.g., methylation, is at a hypomethylated nucleic acid sequence. For example, the chemically modified sequence in the target gene or chromosome region can lack methyl groups on the 5-methyl cytosine nucleotide (e.g., in CpG) as compared to a standard control. Hypomethylation may occur, for example, in aging cells or in cancer (e.g., early stages of neoplasia) relative to the younger cell or non-cancer cell, respectively. In some embodiments, the target polynucleotide sequence is within a CpG island. In some embodiments, the target gene is known to be associated with a disease or condition (e.g., PSCK9). In some embodiments, the target gene comprises a specific copy of disease related sequence. In some embodiments, the target gene harbors the target sequence which is related to a disease.

In some embodiments, the alteration of chemical modification, e.g., methylation, is at a hypermethylated nucleic acid sequence. In some embodiments, the chemical modification is within a CpG island.

In some embodiments, the protein fusion construct has 1 effector domain, 2 effector domains, 4 effector domains, 5 effector domains, 6 effector domains, 7 effector domains, 8 effector domains, 9 effector domains, or 10 effector domains.

### Methyltransferase Domains

In some embodiments, the effector domain comprises a histone methyltransferase domain. Alternatively, the effector domain comprises a recruiter domain which will recruit a subsequent domain with enzymatic activity (e.g., a histone methyltransferase domain). For example, repression (or silencing) can result from repressive chromatin markers, methylation of DNA, methylation of histone residues (e.g., H3K9, H3K27), or deacetylation of histone residues on chromatin containing a target nucleic acid sequence. Without intending to be bound by any theory, the method can be used to change epigenetic state by, for example, closing chromatin via methylation or introducing repressive chromatin markers on chromatin containing the target nuclei acid sequence (e.g., gene).

Specific epigenetic imprints direct gene transcription or gene silencing. For example, DNA methylation, histone modification, repressor proteins binding to silencer regions, and other transcriptional activities alter gene expression without changing the underlying DNA sequence. Thus, the transcriptional regulation allows for expression of specific genes in a particular manner, while repressing other genes. In certain instances, cell fate or function is controlled, either for initial differentiation (e.g., during the organism's development) or to reprogram a cell or cell type (e.g., during disease such as cancer, chronic inflammation, auto-immune disease, illnesses related to various microbiomes of an organism, etc.). Histone modifications play a structural and biochemical role in gene transcription, in one avenue by formation or disruption of the nucleosome structure that binds to the histone and prevents gene transcription. Histones are basic proteins that are commonly found in the nucleus of eukaryotic cells, ranging from multicellular organisms including humans to unicellular organisms represented by fungi (mold and yeast) and ionically bind to genomic DNA. Histones usually consist of five components (H1, H2A, H2B, H3 and H4) and are highly similar across biological species. In the case of histone H4, for example, budding yeast histone H4 (full-length 102 amino acid sequence) and human histone H4 (full-length 102 amino acid sequence) are identical in 92% of the amino acid sequences and differ only in 8 residues. Among the natural proteins assumed to be present in several tens of thousands of organisms, histones are known to be proteins most highly preserved among eukaryotic species. Genomic DNA is folded with histones by ordered binding, and a complex of both forms a basic structural unit called a nucleosome. In addition, aggregation of the nucleosomes forms a chromosomal chromatin structure. Histones are subject to modifications, such as acetylation, methylation, phosphorylation, ubiquitination, SUMOylation and the like, at their N-terminal ends called histone tails, and maintain or specifically convert the chromatin structure, thereby controlling responses such as gene expression, DNA replication, DNA repair and the like, which occur on chromosomal DNA. Post-translational modification of histones is an epigenetic regulatory mechanism and is considered essential for the genetic regulation of eukarvotic cells. Recent studies have revealed that chromatin remodeling factors such as SWI/SNF, RSC, NURF, NRD and the like, which encourage DNA access to transcription factors by modifying the nucleosome structure, histone acetyltransferases (HATs) that regulate the acetylation state of histones, and histone deacetylases (HDACs), act as important regulators. DNA methylation occurs primarily at CpG sites (shorthand for “C-phosphate-G-” or “cytosine-phosphate-guanine”). Highly methylated areas of DNA tend to be less transcriptionally active than lesser methylated sites. Many mammalian genes have promoter regions near or including CpG islands (regions with a high frequency of CpG sites).

In particular, the unstructured N-termini of histones can be modified by at least one of acetylation, methylation, ubiquitylation, phosphorylation, sumoylation, ribosylation, citrullination O-GlcNAcylation, or crotonylation. For example, acetylation of K14 and K9 lysines of histone H3 by histone acetyltransferase enzymes may be linked to transcriptional competence in humans. Lysine acetylation may directly or indirectly create binding sites for chromatin-modifying enzymes that regulate transcriptional activation. For example, histone acetyltransferases (HATs) utilize acetyl-CoA as a cofactor and catalyze the transfer of an acetyl group to the epsilon amino group of the lysine side chains. This neutralizes the lysine's positive charge and weakens the interactions between histones and DNA, thus opening the chromosomes for transcription factors to bind and initiate transcription. Likewise, histone methylation of lysine 9 of histone H3 may be associated with heterochromatin, or transcriptionally silent chromatin. Particular DNA methylation patterns can be established and modified by at least one or more, two or more, three or more, four or more, or five or more independent DNA methyltransferases, including DNMT1, DNMT3A, and DNMT3B.

In some embodiments, the effector domain comprises a histone methyltransferase domain. In some embodiments, the effector domain comprises a DOT1L domain, a SET domain, a SUV39H1 domain, a G9a/EHMT2 protein domain, a EZH1 domain, a EZH2 domain, a SETDB1 domain, or any combination thereof. In some embodiments, the effector domain comprises a histone-lysine-N-methyltransferase SETDB1 domain.

In some embodiments, the effector domain comprises a DNA methyltransferase domain or a Histone methyltransferase domain. DNA methyltransferase domains may mediate methylation at DNA nucleotides, for example at any of an A, T, G or C nucleotide. In some embodiments, the methylated nucleotide is a N6-methyladenosine (m6A). In some embodiments, the methylated nucleotide is a 5-methylcytosine (5mC). In some embodiments, the methylation is at a CG (or CpG) dinucleotide sequence. In some embodiments, the methylation is at a CHG or CHH sequence, where H is any one of A, T, or C.

In some embodiments, the effector domain comprises a DNA methyltransferase DNMT domain that catalyzes transfer of a methyl group to cytosine, thereby repressing expression of the target gene through the recruitment of repressive regulatory proteins. In some embodiments, the effector domain comprises a DNA methyltransferase (DNMT) family protein domain. In some embodiments, the effector domain comprises a DNMT1 domain. In some embodiments, the effector domain comprises a TRDMT1 domain. In some embodiments, the effector domain comprises a DNMT3 domain. In some embodiments, the effector domain comprises a DNMT3A domain. In some embodiments, the effector domain comprises a DNMT3B domain. In some embodiments, the effector domain comprises a DNMT3C domain. In some embodiments, the effector domain comprises a DNMT3L domain. In some embodiments, the effector domain comprises a fusion of DNMT3A-DNMT3L domain.

### Repressor Domains

In some embodiments, the effector domain represses expression of the target gene. Alternatively, or in addition to, in some embodiments, the effector domain recruits one or more protein domains that repress expression of the target gene. In some embodiments, the effector domain interacts with a scaffold protein domain that recruits one or more protein domains that repress expression of the target gene. For example, the effector domain can recruit or interact with a scaffold protein domain that recruits a PRMT protein, a HDAC protein, a SETDB1 protein, or a NuRD protein domain. In some embodiments, the effector domain comprises a Kruppel associated box (KRAB) repression domain: a Repressor Element Silencing Transcription Factor (REST) repression domain, KRAB-associated protein 1 (KAP1) domain, a MAD domain, a FKHR (forkhead in rhabdosarcoma gene) repressor domain, aEGR-1 (early growth response gene product-1) repressor domain, a ets2 repressor factor repressor domain (ERD), a MAD smSIN3 interaction domain (SID), a WRPW motif of the hairy-related basic helix-loop-helix (bHLH) repressor proteins; an HP1 alpha chromo-shadow repression domain, or any combination thereof. In some embodiments, the effector domain comprises a KRAB domain. In some embodiments, the effector domain comprises a Tripartite motif containing 28 (TRIM28, TIF1-beta, or KAP1) protein.

In some embodiments, an effector domain comprises a protein domain that represses expression of the target gene. For example, the effector domain may comprises a functional domain derived from a zinc finger repressor protein. In some embodiments, the effector domain comprises a functional repression domain derived from a KOX1/ZNF10 domain, a KOX8/ZNF708 domain, a ZNF43 domain, a ZNF184 domain, a ZNF91 KRAB domain, a HPF4 domain, a HTF10 domain or a HTF34 domain or any combination thereof. In some embodiments, the effector domain comprises a functional repression domain derived from a ZIM3 protein domain, a ZNF436 domain, a ZNF257 domain, a ZNF675 domain, a ZNF490 domain, a ZNF320 domain, a ZNF331 domain, a ZNF816 domain, a ZNF680 domain, a ZNF41 domain, a ZNF189 domain, a ZNF528 domain, a ZNF543 domain, a ZNF554 domain, a ZNF140 domain, a ZNF610 domain, a ZNF264 domain, a ZNF350 domain, a ZNF8 domain, a ZNF582 domain, a ZNF30 domain, a ZNF324 domain, a ZNF98 domain, a ZNF669 domain, a ZNF677 domain, a ZNF596 domain, a ZNF214 domain, a ZNF37A domain, a ZNF34 domain, a ZNF250 domain, a ZNF547 domain, a ZNF273 domain, a ZNF354A domain, a ZFP82 domain, a ZNF224 domain, a ZNF33A domain, a ZNF45 domain, a ZNF175 domain, a ZNF595 domain, a ZNF184 domain, a ZNF419 domain, a ZFP28-1 domain, a ZFP28-2 domain, a ZNF18 domain, a ZNF213 domain, a ZNF394 domain, a ZFP1 domain, a ZFP14 domain, a ZNF416 domain, a ZNF557 domain, a ZNF566 domain, a ZNF729 domain, a ZIM2 domain, a ZNF254 domain, a ZNF764 domain, a ZNF785 domain or any combination thereof. In some embodiments, the domain is a ZIM3 domain, a ZNF554 domain, a ZNF264 domain, a ZNF324 domain, a ZNF354A domain, a ZNF189 domain, a ZNF543 domain, a ZFP82 domain, a ZNF669 domain, or a ZNF582 domain or any combination thereof. In some embodiments, the domain is a ZIM3 domain, a ZNF554 domain, a ZNF264 domain, a ZNF324 domain, or a ZNF354A domain or any combination thereof. In some embodiments, the domain is a ZIM3 domain.

Sequences of exemplary functional domains that can reduce or silence target gene expression are provided in Table 2 below. Further examples of repressors and repressor domains can be found in PCT/US2021/030643 and Tycko et al. (Tycko J, DelRosso N, Hess G T, Aradhana, Banerjee A, Mukund A, Van M V, Ego B K, Yao D, Spees K, Suzuki P, Marinov G K, Kundaje A, Bassik M C, Bintu L. High-Throughput Discovery and Characterization of Human Transcriptional Effectors. Cell. 2020 Dec. 23; 183(7):2020-2035.e16. doi: 10.1016/j.cell.2020.11.024. Epub 2020 Dec. 15. PMID: 33326746; PMCID: PMC8178797.), which are incorporated here by reference to in its entirety.

In some embodiments, an effector domain comprises a functional domain that represses or silences gene expression, and the functional domain is a part of a larger protein, e.g., a zinc finger repressor protein. Functional domains that are capable of modulating gene expression, e.g., repress or increase gene expression can be identified from the larger protein with known methods and methods provided herein. For example, functional effector domains that can reduce or silence target gene expression may be identified based on sequences of repressor or activator proteins. Amino acid sequences of proteins having the function of modulating gene expression may be obtained from available genome browsers, such as UCSD genome browser or Ensembl genome browser.

Protein annotation databases such as UniProt or Pfam can be used to identify functional domains within the full protein sequence. Using these tools, the repression domain can be identified within the protein sequence. In some instances, various functional domains identified from a larger protein may be tested. Databases may differ in the specific boundary domains. In further embodiments, the starting point region may be truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids at the N-terminus or C-terminus and various truncations may be tested to identify the minimal functional unit.

In some embodiments, the effector domain comprises a Tripartite motif containing 28 (TRIM28, TIF1-beta, or KAP1) protein. In some embodiments, the effector domain comprises one or more KAP1 protein. The KAP1 protein in an epigenetic editing system can form a complex with one or more other effector domains of the epigenetic editing system or one or more proteins involved in modulation of gene expression in a cellular environment. For example, KAP1 may be recruited by a KRAB domain of a transcriptional repressor. In some embodiments, KAP1 interacts with or recruits a histone deacetylase protein, a histone-lysine methyltransferase protein (e.g. depositing methyl groups on lysine 9 [K9] of a histone H3 tail [H3K9]), a chromatin remodeling protein, and/or a heterochromatin protein. In some embodiments, a KAP1 protein interacts with or recruits one or more protein complexes that reduces or silences gene expression. In some embodiments, a KAP1 protein interacts with or recruits a heterochromatin protein 1 (HP1) protein (e.g., via a chromoshadow domain of the HP I protein), a SETDB1 protein, a HDAC protein, and/or a NuRD protein complex component. In some embodiments, a KAP1 protein recruits a CHD3 subunit of the nucleosome remodeling and deacetylation (NuRD) complex, thereby decreasing or silencing expression of a target gene. In some embodiments, a KAP1 protein recruits a SETDB1 protein (e.g. to a promoter region of a target gene), thereby decreasing or silencing expression of the target gene via H3K9 methylation associated with, e.g. the promoter region of the target gene. In some embodiments, recruitment of the SETDB1 protein results in heterochromatinization of a chromosome region harboring the target gene, thereby reducing or silencing expression of the target gene. In some embodiments, a KAP1 protein interacts with or recruits a HP1 protein, thereby decreasing or silencing expression of a target gene via reduced acetylation of H3K9 or H3K14 on histone tails associated with the target gene. Recruitment of SETDB1 induces heterochromatinization. In some embodiments, a KAP1 protein interacts with or recruits a ZFP90 protein (e.g., isoform 2 of ZFP90).

### Effect of Epigenetic Editing System

In some embodiments, the epigenetic editing system (e.g., modified sgRNA complexed with Cas protein-effector domain fusion) disclosed herein results in epigenetic modification. e.g., DNA methylation, in a coding region of the target gene, thereby reducing or silencing expression of the target gene. In some embodiments, the epigenetic editing system results in epigenetic modification, e.g., DNA methylation, in a regulatory sequence such as a promoter or enhancer of the target gene, thereby reducing or silencing expression of the target gene. In some embodiments, the epigenetic editing system results in transcription repression or recruits a transcription repressor to a coding region of the target gene, thereby reducing or silencing expression of the target gene. In some embodiments, the epigenetic editing system recruits a transcription repressor to a regulatory sequence such as a promoter or enhancer of the target gene, thereby reducing or silencing expression of the target gene. In some embodiments, the epigenetic editing system results in epigenetic modification, e.g., DNA demethylation, in a coding region of the target gene, thereby increasing expression of the target gene. In some embodiments, the epigenetic editing system results in epigenetic modification, e.g., DNA demethylation, in a regulatory sequence such as a promoter or enhancer of the target gene, thereby increasing expression of the target gene. In some embodiments, the epigenetic editing system results in transcription activation or recruits a transcription activator to a coding region of the target gene, thereby increasing expression of the target gene. In some embodiments, the epigenetic editing system recruits a transcription activator to a regulatory sequence such as a promoter or enhancer of the target gene, thereby increasing expression of the target gene.

In some embodiments, the target gene and/or the protein (e.g., PCSK9) encoded are associated with a disease, disorder, or pathogenic condition. Alternatively, or in addition to, the target genes do not have a direct associate with a disease but can instead compensate for the disease-associated gene. In some embodiments, the target genes are transcription factors or other genes used to engineer cells.

Epigenetic modifications effected by the epigenetic editing systems described herein are sequence specific. In some embodiments, the modification is at a specific site of the target polynucleotide. In some embodiments, the modification is at a specific allele of the target gene. Accordingly, the epigenetic modification can result in modulated expression, for example, reduced or increased expression, of one copy of a target gene harboring a specific allele, and not the other copy of the target gene. In some embodiments, the specific allele is associated with a disease, condition, or disorder.

Epigenetic modification can be made at any target genes of a genome of interest, for example, a prokaryote genome, a plant genome, a viral genome, mammalian or human genome. The target gene can be of or derived from any organism and genome thereof. For example, the target gene can be a prokaryotic gene, a eukaryotic gene, a viral gene, an animal gene, a plant gene, a mouse gene, a rat gene, a rabbit gene, a fish gene, an avian gene, a monkey gene, or a human gene. In some embodiments, the target gene is a reporter gene the expression of which can be readily tracked and monitored. Reporter genes and reporter systems include, for example, sequences encoding green fluorescence proteins, red fluorescence proteins, enhanced yellow or enhanced cyan proteins, or luciferase proteins. In some embodiments, the target gene encodes a selectable marker, for example, a beta-galactosidase, a Chloramphenicol acetyltransferase, or an antibiotic resistance marker. In some embodiments, the target gene is associated with, or harbors one or more mutations that are associated with a disease, condition, or disorder (e.g., PCSK9).

In some embodiments, an epigenetic editing system provided herein affects an epigenetic modification in a gene that harbors a target sequence. In some embodiments, the epigenetic editing system modulates expression of a protein encoded by the gene. In some embodiments, the epigenetic editing system reduces the level of a protein encoded by the gene. In some embodiments, the epigenetic editing system increases the level of a protein encoded by the gene.

### Methods

Method of Delivery of Modified sgRNA

Disclosed herein is a method of modulating the expression of a target sequence with a composition (e.g., modified sgRNA disclosed herein) and/or components of the epigenetic editing system disclosed herein (e.g., modified sgRNA with a Cas protein, sgRNA with a Cas protein-effector domain fusion). In some embodiments, the method comprises contacting the target sequence or polynucleotide with (i) a sgRNA or a set of sgRNAs disclosed herein, and a (ii) a Cas protein. In some embodiments, the method comprises contacting the target sequence or polynucleotide with (i) a sgRNA or a set of sgRNAs disclosed herein, and a (ii) a Cas protein-effector domain fusion. The target sequence may be epigenetically modified in vivo, ex vivo, or in vitro.

In some embodiments, the sgRNA is introduced into a cell by transfection (e.g., electroporation and lipofection). In some embodiments, electroporation is used to deliver any one of the gRNAs disclosed herein and a Cas protein or an mRNA encoding a Cas protein. In some embodiments, the sgRNA is introduced or delivered into cells via encapsulation by biodegradable polymers, liposomes, nanoparticles, or lipid nanoparticles (LNPs). In some embodiments, the sgRNA is introduced or delivered with LNP. Lipid nanoparticles (LNPs) are a well-known means for delivery of nucleotide and protein cargo, and may be used for delivery of the gRNA, mRNA, a Cas protein, and RNPs disclosed herein. In some embodiments, the LNPs can deliver nucleic acid (e.g., sgRNA), protein, or nucleic acid together with protein. Disclosed herein is a method for delivering any one of the gRNAs disclosed herein to a subject, wherein the gRNA is associated with an LNP. In some embodiments, the gRNA/LNP is also associated with a Cas protein or an mRNA encoding a Cas protein.

In some embodiments, the method comprises a composition comprising sgRNAs disclosed herein and an LNP. In some embodiments, the method comprises a composition further comprising a Cas9 or an mRNA encoding Cas9. In some embodiments, the LNPs comprises cationic lipids. In some embodiments, the LNPs comprises (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)-butanoyl)oxy)-2-((((3-(diethylamino)propoxy)carbonyl)oxy)methyl)propyl (9Z,12Z)-octadeca-9,12-dienoate). In some embodiments, the LNPs comprises molar ratios of a cationic lipid amine to RNA phosphate (N:P) of about 4.5. In some embodiments, LNPs can be associated with the gRNAs disclosed herein, and can be for use in preparing a medicament for treating a disease or disorder. In some embodiments, the method comprises delivering any one of the gRNAs disclosed herein to an ex vivo cell, wherein the gRNA is associated with an LNP or not associated with an LNP. In some embodiments, the gRNA/LNP or gRNA is also associated with a Cas protein or an mRNA encoding a Cas protein.

### Method of Modulating Target Gene

Disclosed herein is a pharmaceutical formulation comprising sgRNA disclosed herein together with a pharmaceutically acceptable carrier. Further disclosed herein is a pharmaceutical formulation comprising sgRNAs disclosed herein and an LNP together with a pharmaceutically acceptable carrier. In some embodiments, the pharmaceutical formulation comprises any one of the sgRNAs disclosed herein, a Cas9 protein or an mRNA encoding a Cas9 protein, and a LNP together with a pharmaceutically acceptable carrier. In some embodiments, the pharmaceutical formulation is for use in preparing a medicament for treating a disease or disorder. In some embodiments, the method of treating a human patient comprises administering any one of the gRNAs or pharmaceutical formulations described herein.

In some embodiment, the method comprises modulating a target DNA comprising, administering or delivering a Cas protein or Cas mRNA and sgRNA disclosed herein. In some embodiments, the modulation is editing of the target gene. In some embodiments, the modulation is a change in expression of the protein encoded by the target gene. In some embodiments, the method or use results in a double-stranded break within the target gene. In some embodiments, the method or use results in formation of indel mutations during non-homologous end joining of the DSB. In some embodiments, the method or use results in an insertion or deletion of nucleotides in a target gene. In some embodiments, the insertion or deletion of nucleotides in a target gene leads to a frameshift mutation or premature stop codon that results in a non-functional protein. In some embodiments, the insertion or deletion of nucleotides in a target gene leads to a knockdown or elimination of target gene expression. In some embodiments, the method or use comprises homology directed repair of a DSB. In some embodiments, the method or use further comprises delivering to the cell a template, wherein at least a part of the template incorporates into a target DNA at or near a double strand break site induced by the Cas protein. In some embodiments, the method or use results in gene modulation. In some embodiments, the gene modulation increases or decreases gene expression, a change in methylation state of DNA, or modification of a histone subunit. In some embodiments, the method or use results in increased or decreased expression of the protein encoded by the target gene or targe sequence.

The efficacy of sgRNA disclosed herein can be tested in vitro and in vivo. In some embodiments, sgRNA disclosed herein results in gene modulation when provided to a cell together with a Cas protein or a Cas protein-effector domain fusion. In some embodiments, the efficacy of sgRNA is measured in in vitro or in vivo assays. In some embodiments, the activity of a Cas RNP comprising a modified sgRNA is compared to the activity of a Cas RNP comprising an unmodified sgRNA.

In some embodiments, the efficiency of a sgRNA disclosed herein in increasing or decreasing target protein expression is determined by measuring the amount of target protein. In some embodiments, the sgRNA disclosed herein, when used as part of an epigenetic editing system disclosed herein, e.g., provided to a cell together with a Cas protein or a Cas protein-effector domain fusion, increases or decreases the amount of protein produced from the targeted gene. In some embodiments, administering the sgRNA disclosed herein to a subject, wherein the sgRNA directs a Cas protein or a Cas protein-effector domain fusion to the gene encoding the target protein, results in the target protein expression to increase or decrease as compared to a sgRNA control that does not target the Cas protein or Cas protein-effector domain fusion to that gene.

In some embodiments, a Cas protein or a Cas protein-effector domain fusion and modified sgRNA described herein show similar, greater, or reduced activity compared to the unmodified sgRNA. In some embodiments, a Cas protein or a Cas protein-effector domain fusion and modified sgRNA described herein show enhanced activity compared to the unmodified sgRNA.

In some embodiments, the activity of modified sgRNA is measured after in vivo dosing of LNPs comprising modified gRNAs and Cas protein or Cas-effector domain fusion or mRNA encoding Cas protein or Cas-effector domain fusion.

In some embodiments, in vivo efficacy of a sgRNA or composition disclosed herein is determined by editing efficacy measured in DNA extracted from tissue (e.g., liver tissue) after administration of sgRNA and Cas protein or Cas-effector domain fusion.

### Kit

In some embodiments, the disclosure provides a kit or system that is used, for example, to carry out a method described herein. In some embodiments, the kit includes (a) a composition disclosed herein (e.g., sgRNA, Cas proteins, effector domain, or any combination thereof) optionally (b) informational material. In some embodiments, the kit includes (a) a population of cells in a liquid suspension or single cell suspension described herein, and, optionally (b) informational material. The informational material may be descriptive, instructional, marketing or other material that relates to the methods described herein.

The informational material of the kits is not limited in its form. In one embodiment, the informational material may include information about production of the composition, date of expiration, batch or production site information, and so forth. In one embodiment, the informational material relates to methods for administering a dosage form of the composition.

In addition to a dosage form of the composition described herein, the kit may include other ingredients, such as a second agent for treating a disease described herein. Alternatively, the other ingredients may be included in the kit, but in different compositions or containers than a pharmaceutical composition described herein. In such embodiments, the kit may include instructions for admixing a pharmaceutical composition described herein and the other ingredients, or for using a pharmaceutical composition described herein together with the other ingredients.

In some embodiments, the kit contains separate containers, dividers or compartments for the composition and informational material. For example, the pharmaceutical composition may be contained in a bottle, vial, or syringe, and the informational material may be contained in a plastic sleeve or packet. In other embodiments, the separate elements of the kit are contained within a single, undivided container. For example, the dosage form of a pharmaceutical composition described herein is contained in a bottle, vial or syringe that has attached thereto the informational material in the form of a label.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: PCSK9 Silencing Via Epigenetic Editing with Modified Guide RNAs in Mice Expressing Transgenic Human PCSK9

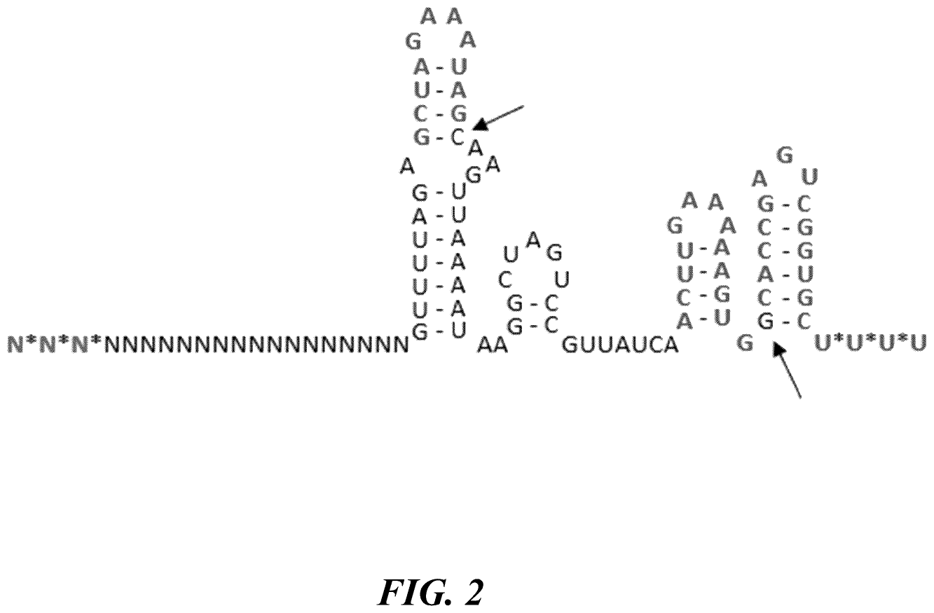

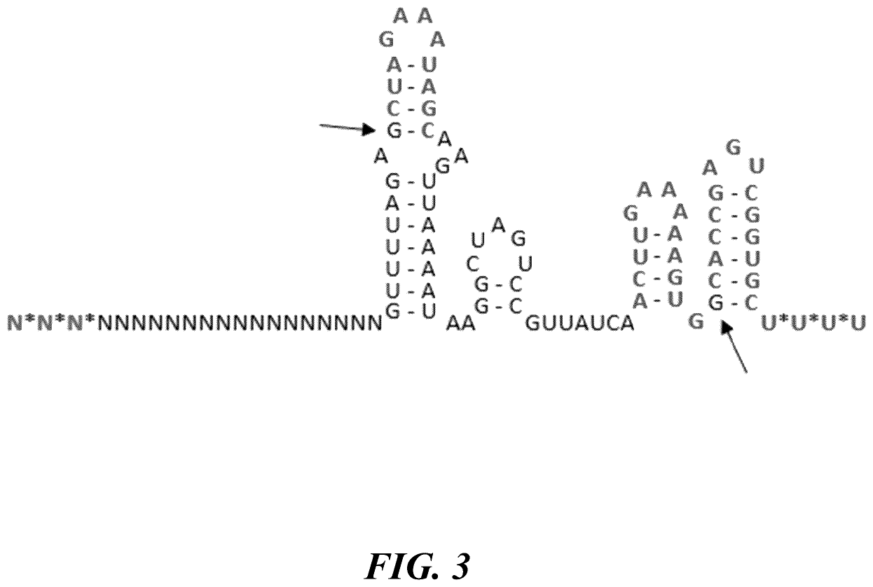

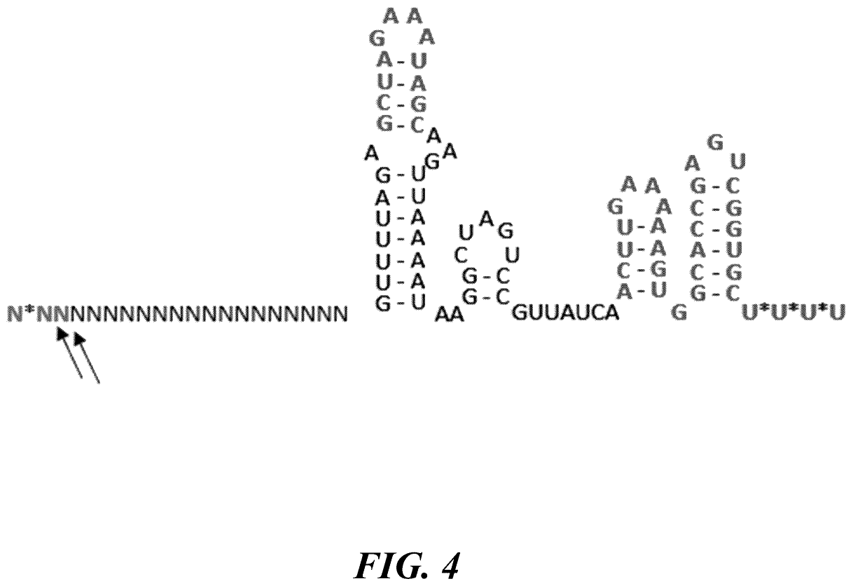

Modified guide RNAs are tested in hPCSK9-Tg (mPCSK9−/−) mice, which express hPCSK9. Guides identified as having PCSK9 targeting activity, with modification patterns representative of those depicted in FIGS. 1, 2, 3, and 4, are applied along with previously tested epigenetic silencers. Constructs are delivered via single IV administration of epigenetic silencer mRNA and gRNA formulated into LNPs in a 1:1 ratio. Efficacy of PCSK9 silencing is measured at 7 days, 14 days, 21 days, 28 days, and 35 days post-IV administration by plasma ELISA for human PCSK9. Robust and durable silencing is observed with all guide RNAs tested.

Durability is tested over a further period of six to twelve months. Readouts are serum or plasma PCSK9 levels and serum or plasma cholesterol levels.

### Example 2: HBV Silencing Via Epigenetic Editing with Modified Guide RNAs in Two In Vivo Models

Two different HBV rodent models are tested in this study. In one set of experiments, a non-transgenic model of persistent HBV infection in immunocompetent mice is used, which was established by administering an adeno-associated viral vector (AAV) that contains HBV Genotype D DNA into the mice. The administration of the AAV-HBV vector resulted in expression of hepatitis B surface antigen (HBsAg), hepatitis B e antigen (HBeAg), and high levels of serum HBV DNA in the mice. In another set of experiments, a transgenic mouse model of persistent HBV infection is used, in which the genome was engineered to integrate HBV Genotype A DNA, resulting in expression of HBsAg and HBeAg, and circulating viral DNA in the mice.

Guides identified as having HBV genome targeting activity, with modification patterns representative of those depicted in FIGS. 1, 2, 3, and 4, are applied along with previously tested epigenetic silencers. Constructs are delivered via single IV administration of epigenetic silencer mRNA and gRNA formulated into LNPs in a 1:1 ratio. Efficacy of HBV silencing is measured by one or more of measurement of HBsAg antigen, HBeAg antigen, or HBV DNA in serum at 7 days, 14 days, 21 days, 28 days, and 35 days post-V administration. Silencing is observed with all guide RNAs tested in both murine models. Durability is tested over a further period of six to twelve months.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1.-80. (canceled)

81. A single guide RNA (sgRNA) comprising a tracr sequence and a spacer sequence,
(i) wherein the tracr sequence comprises an upper stem region, a hairpin region 1, and a hairpin region 2, and wherein
(a) at least 50% of the nucleotides in the upper stem region are 2′-O-Me modified, and at least one nucleotide in the upper stem region is not 2′-O-Me modified; and
(b) at least 50% of the nucleotides in the hairpin region 2 are 2′-O-Me modified, and at least one nucleotide in the hairpin region 2 is not 2′-O-Me modified;
or
(ii) wherein the spacer sequence comprises a 5′ end, and wherein the spacer sequence comprises not more than one phosphorothioate (PS) bond within the first three nucleotides of the 5′ end.

82. The sgRNA of claim 81, wherein each nucleotide in the upper stem region is modified with 2′-O-Me except a first nucleotide or a last nucleotide of the upper stem region in the 5′ end to 3′ end direction.

83. The sgRNA of claim 81, wherein the upper stem region, hairpin region 1, or hairpin region 2 further comprises nucleotides modified with 2′-deoxy (2′-H), 2-MOE, 2′-F, 2′-NH₂, 2′-arabinosyl(2-arabino) nucleotide, 2-F-arabinosyl(2′-F-arabino) nucleotide, 2′-locked nucleic acid (LNA) nucleotide, 2′-unlocked nucleic acid (ULNA) nucleotide, a sugar in L form (L-sugar), 4′-thioribosyl nucleotide, or any combination thereof.

84. The sgRNA of claim 81, wherein at least 80% of the nucleotides in the upper stem region are modified.

85. The sgRNA of claim 81, wherein the upper stem region, hairpin region 1, or hairpin region 2 comprises a linkage modification selected from phosphorothioate, phosphonoacetate, thiophosphonoacetate, methylphosphonoate-P(CH₃), boranophosphonate, phosphorodithioate, or any combination thereof.

86. The sgRNA of claim 81, wherein each nucleotide in the hairpin region 2 is modified with 2′-O-Me except a first nucleotide in the 5′ end to 3′ end direction.

87. The sgRNA of claim 81, wherein at least 60% of nucleotides in the hairpin region 2 are modified.

88. The sgRNA of claim 81, further comprising a 5′ terminus, wherein the 5′ terminus comprises the PS bond between a first and second nucleotides and at least 50% of nucleotides of the 5′ terminus are modified.

89. The sgRNA of claim 88, wherein the 5′ terminus comprises nucleotides modified with 2′-O-Me, 2′-deoxy (2′-H), 2-MOE, 2′-F, 2′-NH₂, 2′-arabinosyl(2-arabino) nucleotide, 2-F-arabinosyl(2′-F-arabino) nucleotide, 2′-locked nucleic acid (LNA) nucleotide, 2′-unlocked nucleic acid (ULNA) nucleotide, a sugar in L form (L-sugar), 4′-thioribosyl nucleotide, or any combination thereof.

90. The sgRNA of claim 81, wherein each nucleotide in the hairpin region 1 is modified with 2′-O-Me.

91. The sgRNA of claim 81, wherein at least 50% of nucleotides in the hairpin region 1 are modified.

92. The sgRNA of claim 81, further comprising a 3′ terminus, wherein at least two of the last four nucleotides at the 3′ end of the 3′ terminus are modified and wherein the last four nucleotides at the 3′ end of the 3′ terminus are linked with PS bonds.

93. The sgRNA of claim 81, further comprising a lower stem region, a bulge region, and a nexus region wherein each region comprises one or more modifications selected from the group consisting of: 2′-O-Me, 2′-deoxy (2′-H), 2-MOE, 2′-F, 2′-NH2, 2′-arabinosyl(2-arabino) nucleotide, 2′-F-arabinosyl(2′-F-arabino) nucleotide, 2′-locked nucleic acid (LNA) nucleotide, 2′-unlocked nucleic acid (ULNA) nucleotide, a sugar in L form (L-sugar), 4′-thioribosyl nucleotide, and combinations thereof.

94. The sgRNA of claim 92, wherein last four nucleotides of the 3′ end of the 3′ terminus are modified with 2′-O-Me and wherein a 5′ end of a 5′ terminus comprises first four nucleotides linked with the PS bond.

95. The sgRNA of claim 81, wherein the sgRNA comprises a sequence set forth in any one of SEQ ID NOs: 1-14, or 18-34.

96. A single guide RNA (sgRNA) comprising:
(i) an upper stem region, a hairpin region 1, a hairpin region 2, wherein
(a) the upper stem region comprises nucleotides modified with 2′-O-Me;
(b) the hairpin region 1 and the hairpin region 2 comprise nucleotides modified with 2′-O-Me; or
(c) both (a) and (b); and
(ii) a 5′ terminus, wherein the 5′ terminus targets hPCSK9.

97. A compound of Formula (I):
X_{A1}X_{A2}X_{A3}(Xᵢ)_{M}X_{B1}X_{B2}X_{B3}X_{B4}X_{B5}X_{B6}X_{C1}X_{C2}X_{D1}X_{D2}X_{D3}X_{D4}X_{D5}X_{D6}X_{D7}X_{D8}
X_{D9}X_{D10}X_{D11}X_{D12}X_{C3}X_{C4}X_{C5}X_{C6}X_{B7}X_{B8}X_{B9}X_{B10}X_{B11}X_{B12}X_{E1}X_{E2}X_{E3}X_{E4}X_{E5}
X_{E6}X_{E7}X_{E8}X_{E9}X_{E10}X_{E11}X_{E12}
X_{E13}X_{E14}X_{E15}X_{E16}X_{E17}X_{E18}X_{F1}X_{F2}X_{F3}X_{F4}X_{F5}X_{F6}X_{F7}X_{F8}X_{F9}X_{F10}X_{F11}X_{F12}
X_{G1}X_{H1}X_{H2}X_{H3}X_{H4}X_{H5}X_{H6}X_{H7}X_{H8}X_{H9}X_{H10}X_{H11}X_{H12}X_{H13}X_{H14}X_{H15}X_{J1}X_{J2}X_{J3}X_{J4}
(I),
wherein:
each of X_{B6}, X_{B8}, X_{B9}, X_{B10}, X_{B11}, X_{C2}, X_{C3}, X_{C4}, X_{E1}, X_{E2}, X_{E7}, X_{E15}, and X_{E18} is 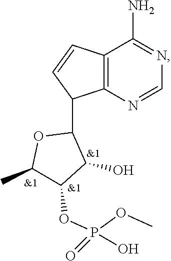 [SMILES: COP(=O)(O)O[C@@H]1[C@@H](C)OC(C2C=Cc3c(N)ncnc32)[C@@H]1O]
each of X_{E5}, X_{E10}, X_{E11}, X_{E17}, is 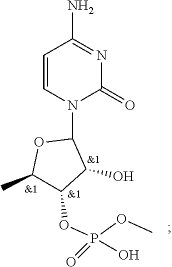
each of X_{B1}, X_{C1}, X_{C5}, X_{E3}, X_{E4}, X_{E8}, and X_{E12} is 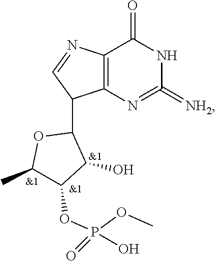
and
each of X_{B2}, X_{B3}, X_{B4}, X_{B5}, X_{B7}, X_{B12}, X_{C6}, X_{E6}, X_{E9}, X_{E13}, X_{E14}, X_{E16} is 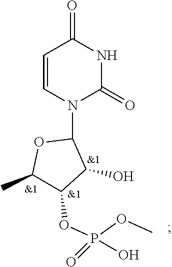
wherein:
each of X_{F1}, X_{F6}, X_{F7}, X_{F8}, X_{F9}, X_{F10}, is 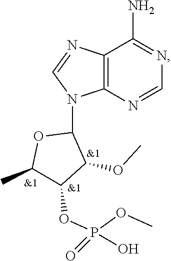 [SMILES: CO[C@H]1C(n2cnc3c(N)ncnc32)O[C@H](C)[C@H]1OP(=O)(O)OC]
X_{F2} is 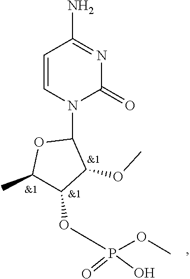
each of X_{F5}, X_{F11}, X_{G1} is 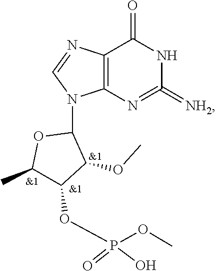
and
each of X_{F3}, X_{F4}, X_{F12} is 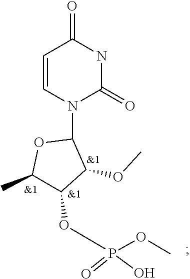
wherein:
each of X_{D4}, X_{D6}, X_{D7}, X_{D8}, X_{D10}, X_{H3}, and X_{H7} is 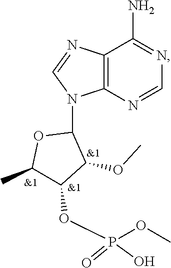
each of X_{D2}, X_{H2}, X_{H4}, X_{H5}, X_{H10}, and X_{H15} is 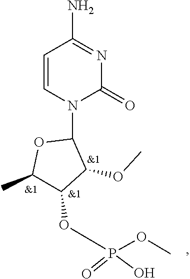
each of X_{D5}, X_{D11}, X_{H6}, X_{H8}, X_{H11}, X_{H12}, and X_{H14} is 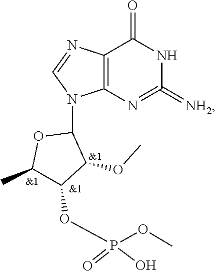
each of X_{D3}, X_{D9}, X_{H9}, and X_{H13} is 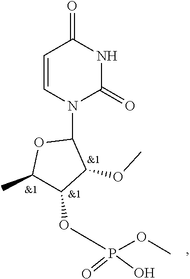
each of X_{J1} and X_{J2}, and X_{J3} is 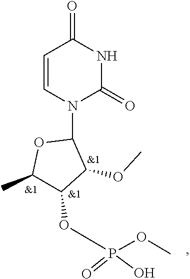
and
X_{J4} is 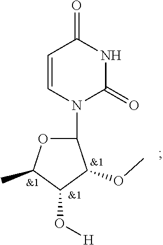
wherein:
X_{A1} is 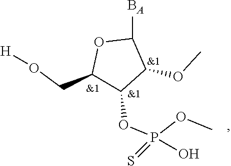
M is 14, 15, 16, 17, 18, 19, 20, or 21,
Xᵢ is 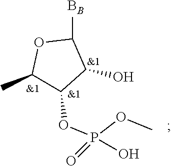
wherein:
(a) X_{A2} and X_{A3} are both 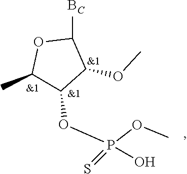
X_{D1} is 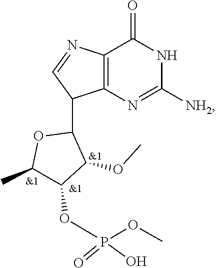 [SMILES: CO[C@H]1C(C2C=Nc3c2nc(N)[nH]c3=O)O[C@H](C)[C@H]1OP(=O)(O)OC]
X_{D12} is 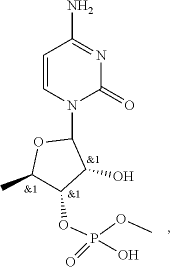
and
X_{D12} is 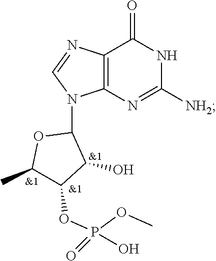 [SMILES: COP(=O)(O)O[C@@H]1[C@@H](C)OC(n2cnc3c(=O)[nH]c(N)nc32)[C@@H]1O]
(b) X_{A2} and X_{A3} are both 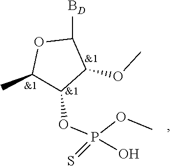
X_{D1} is 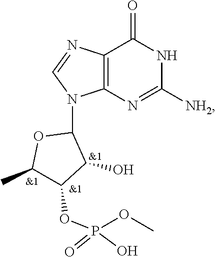 [SMILES: COP(=O)(O)O[C@@H]1[C@@H](C)OC(n2cnc3c(=O)[nH]c(N)nc32)[C@@H]1O]
X_{D12} is 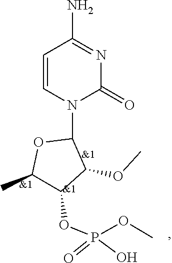
and X_{D12} is 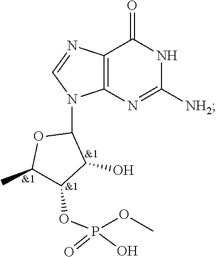 [SMILES: COP(=O)(O)O[C@@H]1[C@@H](C)OC(n2cnc3c(=O)[nH]c(N)nc32)[C@@H]1O]
or
(c) X_{A2} and X_{A3} are both 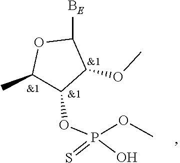
X_{D1} and X_{H1} are both 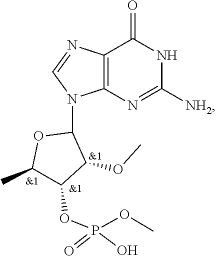 [SMILES: CO[C@H]1C(n2cnc3c(=O)[nH]c(N)nc32)O[C@H](C)[C@H]1OP(=O)(O)OC]
and X_{D12} is 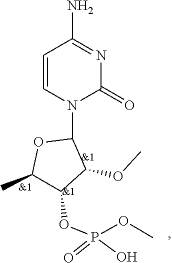
wherein each of B_{A}, B_{B}, B_{C}, B_{D}, and B_{E} is independently selected from the group consisting of:

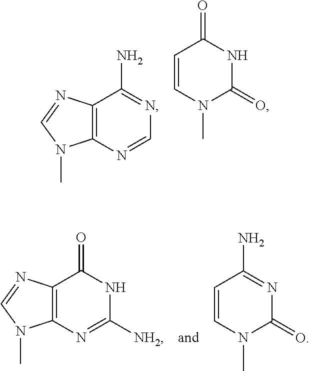

98. An epigenetic system comprising a nuclease, and the sgRNA of claim 81.

99. A method, comprising administering the sgRNA of claim 81 to a cell.

100. A method, comprising administering the sgRNA of claim 81 to a subject in need thereof.


SEQUENCE LISTING (51 sequences)

SEQ ID NO: 1 - RNA, 20 nt, synthetic construct
  source 1..20: mol_type = other RNA
actgcctggctcactcctcc

SEQ ID NO: 2 - RNA, 20 nt, synthetic construct
  source 1..20: mol_type = other RNA
tgcggaaaccttctagggtg

SEQ ID NO: 3 - RNA, 20 nt, synthetic construct
  source 1..20: mol_type = other RNA
atcgtccgatggggctctgg

SEQ ID NO: 4 - RNA, 20 nt, synthetic construct
  source 1..20: mol_type = other RNA
gcggaaaccttctagggtgt

SEQ ID NO: 5 - RNA, 20 nt, synthetic construct
  source 1..20: mol_type = other RNA
gtgtgggtgcttgacgcctg

SEQ ID NO: 6 - RNA, 20 nt, synthetic construct
  source 1..20: mol_type = other RNA
  modified_base 1..3: mod_base = OTHER; note = 2'-O-Methyl modified nucleotide phosphorothioate
actgcctggctcactcctcc

SEQ ID NO: 7 - RNA, 20 nt, synthetic construct
  source 1..20: mol_type = other RNA
  modified_base 1..3: mod_base = OTHER; note = 2'-O-Methyl modified nucleotide phosphorothioate
tgcggaaaccttctagggtg

SEQ ID NO: 8 - RNA, 20 nt, synthetic construct
  source 1..20: mol_type = other RNA
  modified_base 1..3: mod_base = OTHER; note = 2'-O-Methyl modified nucleotide phosphorothioate
atcgtccgatggggctctgg

SEQ ID NO: 9 - RNA, 20 nt, synthetic construct
  source 1..20: mol_type = other RNA
  modified_base 1..3: mod_base = OTHER; note = 2'-O-Methyl modified nucleotide phosphorothioate
gcggaaaccttctagggtgt

SEQ ID NO: 10 - RNA, 20 nt, synthetic construct
  source 1..20: mol_type = other RNA
  modified_base 1..3: mod_base = OTHER; note = 2'-O-Methyl modified nucleotide phosphorothioate
gtgtgggtgcttgacgcctg

SEQ ID NO: 11 - RNA, 20 nt, synthetic construct
  source 1..20: mol_type = other RNA
  modified_base 1..3: mod_base = OTHER; note = 2'-O-Methyl modified nucleotide
  modified_base 1..2: mod_base = OTHER; note = Phosphorothioate linkage
actgcctggctcactcctcc

SEQ ID NO: 12 - RNA, 20 nt, synthetic construct
  source 1..20: mol_type = other RNA
  modified_base 1..3: mod_base = OTHER; note = 2'-O-Methyl modified nucleotide
  modified_base 1..2: mod_base = OTHER; note = Phosphorothioate linkage
tgcggaaaccttctagggtg

SEQ ID NO: 13 - RNA, 20 nt, synthetic construct
  source 1..20: mol_type = other RNA
  modified_base 1..3: mod_base = OTHER; note = 2'-O-Methyl modified nucleotide
  modified_base 1..2: mod_base = OTHER; note = Phosphorothioate linkage
atcgtccgatggggctctgg

SEQ ID NO: 14 - RNA, 20 nt, synthetic construct
  source 1..20: mol_type = other RNA
  modified_base 1..3: mod_base = OTHER; note = 2'-O-Methyl modified nucleotide
  modified_base 1..2: mod_base = OTHER; note = Phosphorothioate linkage
gcggaaaccttctagggtgt

SEQ ID NO: 15 - RNA, 80 nt, synthetic construct
  source 1..80: mol_type = other RNA
  modified_base 9..19: mod_base = OTHER; note = 2'-O-Methyl modified nucleotide
  modified_base 49..61: mod_base = OTHER; note = 2'-O-Methyl modified nucleotide
  modified_base 63..80: mod_base = OTHER; note = 2'-O-Methyl modified nucleotide
  modified_base 77..80: mod_base = OTHER; note = Phosphorothioate linkage
gttttagagctagaaatagcaagttaaaataaggctagtccgttatcaacttgaaaaagtggcaccgagtcggtgctttt

SEQ ID NO: 16 - RNA, 80 nt, synthetic construct; identical to SEQ ID NO: 15
  source 1..80: mol_type = other RNA
  modified_base 10..20: mod_base = OTHER; note = 2'-O-Methyl modified nucleotide
  modified_base 49..61: mod_base = OTHER; note = 2'-O-Methyl modified nucleotide
  modified_base 63..80: mod_base = OTHER; note = 2'-O-Methyl modified nucleotide
  modified_base 77..80: mod_base = OTHER; note = Phosphorothioate linkage

SEQ ID NO: 17 - RNA, 80 nt, synthetic construct; identical to SEQ ID NO: 15
  source 1..80: mol_type = other RNA
  modified_base 9..20: mod_base = OTHER; note = 2'-O-Methyl modified nucleotide
  modified_base 49..80: mod_base = OTHER; note = 2'-O-Methyl modified nucleotide
  modified_base 77..80: mod_base = OTHER; note = Phosphorothioate linkage

SEQ ID NO: 18 - RNA, 100 nt, synthetic construct
  source 1..100: mol_type = other RNA
  modified_base 1..3: mod_base = OTHER; note = 2'-O-Methyl modified nucleotide phosphorothioate
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage
actgcctggctcactcctccgttttagagctagaaatagcaagttaaaataaggctagtccgttatcaacttgaaaaagtggcaccgagtcggtgctttt

SEQ ID NO: 19 - RNA, 100 nt, synthetic construct
  source 1..100: mol_type = other RNA
  modified_base 1..3: mod_base = OTHER; note = 2'-O-Methyl modified nucleotide phosphorothioate
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage
tgcggaaaccttctagggtggttttagagctagaaatagcaagttaaaataaggctagtccgttatcaacttgaaaaagtggcaccgagtcggtgctttt

SEQ ID NO: 20 - RNA, 100 nt, synthetic construct
  source 1..100: mol_type = other RNA
  modified_base 1..3: mod_base = OTHER; note = 2'-O-Methyl modified nucleotide phosphorothioate
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage
atcgtccgatggggctctgggttttagagctagaaatagcaagttaaaataaggctagtccgttatcaacttgaaaaagtggcaccgagtcggtgctttt

SEQ ID NO: 21 - RNA, 100 nt, synthetic construct
  source 1..100: mol_type = other RNA
  modified_base 1..3: mod_base = OTHER; note = 2'-O-Methyl modified nucleotide phosphorothioate
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage
gcggaaaccttctagggtgtgttttagagctagaaatagcaagttaaaataaggctagtccgttatcaacttgaaaaagtggcaccgagtcggtgctttt

SEQ ID NO: 22 - RNA, 100 nt, synthetic construct
  source 1..100: mol_type = other RNA
  modified_base 1..3: mod_base = OTHER; note = 2'-O-Methyl modified nucleotide phosphorothioate
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage
gtgtgggtgcttgacgcctggttttagagctagaaatagcaagttaaaataaggctagtccgttatcaacttgaaaaagtggcaccgagtcggtgctttt

SEQ ID NO: 23 - RNA, 100 nt, synthetic construct; identical to SEQ ID NO: 18
  source 1..100: mol_type = other RNA
  modified_base 1..3: mod_base = OTHER; note = 2'-O-Methyl modified nucleotide phosphorothioate
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 24 - RNA, 100 nt, synthetic construct; identical to SEQ ID NO: 19
  source 1..100: mol_type = other RNA
  modified_base 1..3: mod_base = OTHER; note = 2'-O-Methyl modified nucleotide phosphorothioate
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 25 - RNA, 100 nt, synthetic construct; identical to SEQ ID NO: 20
  source 1..100: mol_type = other RNA
  modified_base 1..3: mod_base = OTHER; note = 2'-O-Methyl modified nucleotide phosphorothioate
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 26 - RNA, 100 nt, synthetic construct; identical to SEQ ID NO: 21
  source 1..100: mol_type = other RNA
  modified_base 1..3: mod_base = OTHER; note = 2'-O-Methyl modified nucleotide phosphorothioate
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 27 - RNA, 100 nt, synthetic construct; identical to SEQ ID NO: 22
  source 1..100: mol_type = other RNA
  modified_base 1..3: mod_base = OTHER; note = 2'-O-Methyl modified nucleotide phosphorothioate
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 28 - RNA, 100 nt, synthetic construct; identical to SEQ ID NO: 18
  source 1..100: mol_type = other RNA
  modified_base 1..3: mod_base = OTHER; note = 2'-O-Methyl modified nucleotide
  modified_base 1..2: mod_base = OTHER; note = Phosphorothioate linkage
  modified_base 29..40: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 29 - RNA, 100 nt, synthetic construct; identical to SEQ ID NO: 19
  source 1..100: mol_type = other RNA
  modified_base 1..3: mod_base = OTHER; note = 2'-O-Methyl modified nucleotide
  modified_base 1..2: mod_base = OTHER; note = Phosphorothioate linkage
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 30 - RNA, 100 nt, synthetic construct; identical to SEQ ID NO: 20
  source 1..100: mol_type = other RNA
  modified_base 1..3: mod_base = OTHER; note = 2'-O-Methyl modified nucleotide
  modified_base 1..2: mod_base = OTHER; note = Phosphorothioate linkage
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 31 - RNA, 100 nt, synthetic construct; identical to SEQ ID NO: 21
  source 1..100: mol_type = other RNA
  modified_base 1..3: mod_base = OTHER; note = 2'-O-Methyl modified nucleotide
  modified_base 1..2: mod_base = OTHER; note = Phosphorothioate linkage
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 32 - RNA, 100 nt, synthetic construct
  source 1..100: mol_type = other RNA
  modified_base 1..3: mod_base = OTHER; note = 2'-O-Methyl modified nucleotide phosphorothioate
  modified_base 29..39: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage
nnnnnnnnnnnnnnnnnnnngttttagagctagaaatagcaagttaaaataaggctagtccgttatcaacttgaaaaagtggcaccgagtcggtgctttt

SEQ ID NO: 33 - RNA, 100 nt, synthetic construct; identical to SEQ ID NO: 32
  source 1..100: mol_type = other RNA
  modified_base 1..3: mod_base = OTHER; note = 2'-O-Methyl modified nucleotide phosphorothioate
  modified_base 30..40: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 34 - RNA, 100 nt, synthetic construct; identical to SEQ ID NO: 32
  source 1..100: mol_type = other RNA
  modified_base 1..3: mod_base = OTHER; note = 2'-O-Methyl modified nucleotide
  modified_base 1..2: mod_base = OTHER; note = Phosphorothioate linkage
  modified_base 29..40: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 35 - RNA, 100 nt, synthetic construct; identical to SEQ ID NO: 18
  source 1..100: mol_type = other RNA
  modified_base 1..3: mod_base = OTHER; note = 2'-O-Methyl modified nucleotide phosphorothioate
  modified_base 29..40: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 36 - RNA, 100 nt, synthetic construct; identical to SEQ ID NO: 20
  source 1..100: mol_type = other RNA
  modified_base 1..3: mod_base = OTHER; note = 2'-O-Methyl modified nucleotide phosphorothioate
  modified_base 29..40: mod_base = OTHER; note = 2'-O-Methyl modified nucleotide
  modified_base 69..100: mod_base = OTHER; note = 2'-O-Methyl modified nucleotide
  modified_base 97..100: mod_base = OTHER; note = Phosphorothioate linkage

SEQ ID NO: 37 - protein, 2326 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSELEDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVGVDGSSGSETPGTSESATPESTGMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLSAPKKKRKVPKKKRKV

SEQ ID NO: 38 - DNA, 10942 bp, synthetic construct
  source 1..10942: mol_type = other DNA
gggcgctcgagcaggttcagaaggagatcaaaaacccccaaggatcaaacatgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtgggagtcgacggatccagcggctccgagaccccaggcacatctgagagcgccacccctgagtccaccggtatgaacaattcacaggggagagtgacattcgaagacgtgaccgtgaacttcacccagggagaatggcagcgcttgaacccagaacaaaggaacctctatcgggacgtgatgctggaaaactactcaaatttggtgagcgttgggcagggtgagaccactaagcctgacgtgatcctgagattggaacagggcaaggagccttggctcgaggaagaggaagtcctgggctcagggagggccgagaaaaacggtgatataggaggccagatatggaagcctaaggacgtcaaggagagcctgagcgctcccaagaagaaaaggaaggtcccaaagaaaaaaagaaaggtgtgaggatcctgagtctagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagcttgaagagcctagtggcgcctgatgcggtattttctccttacgcatctgtgcggtatttcacaccgcataatccagcacagtggcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtcgatcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataag

SEQ ID NO: 39 - protein, 2326 aa, synthetic construct; identical to SEQ ID NO: 37

SEQ ID NO: 40 - DNA, 10899 bp, synthetic construct
  source 1..10899: mol_type = other DNA
gggcgctcgagcaggttcagaaggagatcaaaaacccccaaggatcaaacatgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtgggagtcgacggatccagcggctccgagaccccaggcacatctgagagcgccacccctgagtccaccggtatgaacaattcacaggggagagtgacattcgaagacgtgaccgtgaacttcacccagggagaatggcagcgcttgaacccagaacaaaggaacctctatcgggacgtgatgctggaaaactactcaaatttggtgagcgttgggcagggtgagaccactaagcctgacgtgatcctgagattggaacagggcaaggagccttggctcgaggaagaggaagtcctgggctcagggagggccgagaaaaacggtgatataggaggccagatatggaagcctaaggacgtcaaggagagcctgagcgctcccaagaagaaaaggaaggtcccaaagaaaaaaagaaaggtgtgaggatcctgagtctagaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaagcttgaagagcctagtggcgcctgatgcggtattttctccttacgcatctgtgcggtatttcacaccgcataatccagcacagtggcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtcgatcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataag

SEQ ID NO: 41 - DNA, 79 bp, synthetic construct
  source 1..79: mol_type = other DNA
gttttagagctagaaatagcaagttaaaataaggctagtccgttatcaacttgaaaaagtggcaccgagtcggtgcttt

SEQ ID NO: 42 - DNA, 80 bp, synthetic construct
  source 1..80: mol_type = other DNA
gtttaagagctagaaatagcaagtttaaataaggctagtccgttatcaacttgaaaaagtggcaccgagtcggtgctttt

SEQ ID NO: 43 - DNA, 93 bp, synthetic construct
  source 1..93: mol_type = other DNA
gtttaagagctaagctggaaacagcatagcaagtttaaataaggctagtccgttatcaacttgaaaaagtggcaccgagtcggtgcttttttt

SEQ ID NO: 44 - DNA, 80 bp, synthetic construct
  source 1..80: mol_type = other DNA
gttttagtactctggaaacagaatctactaaaacaaggcaaaatgccgtgtttatctcgtcaacttgttggcgagatttt

SEQ ID NO: 45 - RNA, 92 nt, synthetic construct
  source 1..92: mol_type = other RNA
gtttaagagctaagctggaaacagcatagcaagtttaaataaggctagtccgttatcaacttgaaaaagtggcaccgagtcggtgctttttt

SEQ ID NO: 46 - RNA, 80 nt, synthetic construct; identical to SEQ ID NO: 15
  source 1..80: mol_type = other RNA

SEQ ID NO: 47 - protein, 4 aa, unidentified
  source 1..4: note = motif of the hairy-related basic helix-loop-helix (bHLH) repressor proteins
WRPW

SEQ ID NO: 48 - RNA, 100 nt, synthetic construct; identical to SEQ ID NO: 32
  source 1..100: mol_type = other RNA
  modified_base 1..2: mod_base = OTHER; note = Phosphorothioate linkage
  modified_base 97..100: mod_base = OTHER; note = Phosphorothioate linkage

SEQ ID NO: 49 - RNA, 100 nt, synthetic construct; identical to SEQ ID NO: 32
  source 1..100: mol_type = other RNA
  modified_base 1..39: mod_base = OTHER; note = 2'-O-Methyl modified nucleotide
  modified_base 1..4: mod_base = OTHER; note = Phosphorothioate linkage
  modified_base 41..81: mod_base = OTHER; note = 2'-O-Methyl modified nucleotide
  modified_base 83..100: mod_base = OTHER; note = 2'-O-Methyl modified nucleotide
  modified_base 97..100: mod_base = OTHER; note = Phosphorothioate linkage

SEQ ID NO: 50 - RNA, 100 nt, synthetic construct; identical to SEQ ID NO: 32
  source 1..100: mol_type = other RNA
  modified_base 1..28: mod_base = OTHER; note = 2'-O-Methyl modified nucleotide
  modified_base 1..4: mod_base = OTHER; note = Phosphorothioate linkage
  modified_base 30..81: mod_base = OTHER; note = 2'-O-Methyl modified nucleotide
  modified_base 83..100: mod_base = OTHER; note = 2'-O-Methyl modified nucleotide
  modified_base 97..100: mod_base = OTHER; note = Phosphorothioate linkage

SEQ ID NO: 51 - RNA, 100 nt, synthetic construct; identical to SEQ ID NO: 32
  source 1..100: mol_type = other RNA
  modified_base 1..2: mod_base = OTHER; note = Phosphorothioate linkage
  modified_base 97..100: mod_base = OTHER; note = Phosphorothioate linkage